(19) United States
(12) Patent Application Publication
(10) Pub. No.: US 2024/0336566 A1
(43) Pub. Date: Oct. 10, 2024

(54) CRYSTALLINE FORMS OF A COMPOUND FOR THE TARGETED DEGRADATION OF THE ANDROGEN RECEPTOR

(71) Applicant: Arvinas Operations, Inc., New Haven, CT (US)

(72) Inventors: Chungpin Herman Chen, Madison, CT (US); Maxwell Marco Reeve, Guilford, CT (US); Nitinchandra Dahyabhai Patel, Robbinsville, NJ (US); Royal J. Haskell, III, Durham, CT (US)

(21) Appl. No.: 18/622,152

(22) Filed: Mar. 29, 2024

Related U.S. Application Data
(60) Provisional application No. 63/493,245, filed on Mar. 30, 2023.

(51) Int. Cl. C07D 211/88

(52) U.S. Cl. CPC C07D 211/88

(57) ABSTRACT

The present disclosure relates to novel solid forms, including salts and solid forms thereof, of Compound A:

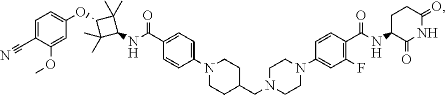 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and to processes for their preparation. The disclosure is also directed to pharmaceutical compositions containing at least one salt or salt form and to the therapeutic and/or prophylactic use of such salts, salt forms, and compositions thereof.

### RELATED APPLICATIONS

This application claims priority to, and the benefit of, U.S. Provisional Application No. 63/493,245, filed Mar. 30, 2023, which is incorporated by reference in its entirety for all purposes.

### TECHNICAL FIELD

The disclosure provides novel salts and salt forms of Compound A:

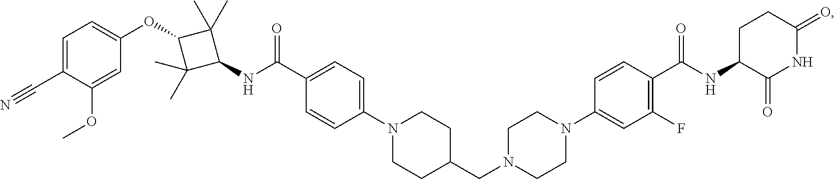 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and to processes for their preparation. The disclosure is also directed to pharmaceutical compositions containing at least one salt or salt form and to the therapeutic and/or prophylactic use of such salts, salt forms, and compositions thereof. These salts and salt forms are useful as modulators of targeted ubiquitination, especially with respect to a variety of polypeptides and other proteins, which are degraded and/or otherwise inhibited by the salts and salt forms of the present disclosure.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and are therefore attractive therapeutic targets. The development of ligands of E3 ligases has proven challenging, in part due to the fact that they must disrupt protein-protein interactions. However, recent developments have provided specific ligands which bind to these ligases.

One E3 ubiquitin ligase with therapeutic potential is cereblon. Cereblon is a protein that in humans is encoded by the CRBN gene. Thalidomide and its analogs, e.g., pomalidomide and lenalidomide, are known to bind cereblon. These agents bind to cereblon, altering the specificity of the complex to induce the ubiquitination and degradation of transcription factors essential for multiple myeloma growth. Indeed, higher expression of cereblon has been linked to an increase in efficacy of imide drugs in the treatment of multiple myeloma.

Androgen Receptor (AR) belongs to a nuclear hormone receptor family that is activated by androgens, such as testosterone and dihydrotestosterone (Pharmacol. Rev. 2006, 58 (4), 782-97; Vitam. Horn. 1999, 55:309-52.). In the absence of androgens, AR is bound by Heat Shock Protein 90 (Hsp90) in the cytosol. When an androgen binds AR, its conformation changes to release AR from Hsp90 and to expose the Nuclear Localization Signal (NLS). The latter enables AR to translocate into the nucleus where AR acts as a transcription factor to promote gene expression responsible for male sexual characteristics (Endocr. Rev. 1987, 8 (1): 1-28; Mol. Endocrinol. 2002, 16 (10), 2181-7). AR deficiency leads to Androgen Insensitivity Syndrome, formerly termed testicular feminization.

While AR is responsible for development of male sexual characteristics, it is also a well-documented oncogene in certain forms of cancers including prostate cancers (Endocr. Rev. 2004, 25 (2), 276-308). A commonly measured target gene of AR activity is the secreted Prostate Specific Antigen (PSA) protein. The current treatment regimen for prostate cancer involves inhibiting the androgen-AR axis by two methods. The first approach relies on reduction of androgens, while the second strategy aims to inhibit AR function (Nat. Rev. Drug Discovery, 2013, 12, 823-824). Despite the development of effective targeted therapies, most patients develop resistance and the disease progresses. An alternative approach for the treatment of prostate cancer involves eliminating the AR protein.

Because AR is a critical driver of tumorigenesis in many forms of prostate cancers, its elimination should lead to a therapeutically beneficial response. There exists an ongoing need in the art for effective treatments for diseases, especially cancer, prostate cancer, and Kennedy's Disease.

However, non-specific effects, and the inability to target and modulate certain classes of proteins altogether, such as transcription factors, remain as obstacles to the development of effective anti-cancer agents. As such, small molecule therapeutic agents that leverage or potentiate cereblon's substrate specificity and, at the same time, are “tunable” such that a wide range of protein classes can be targeted and modulated with specificity would be very useful as a therapeutic.

### SUMMARY

The present disclosure is directed to Compound A:

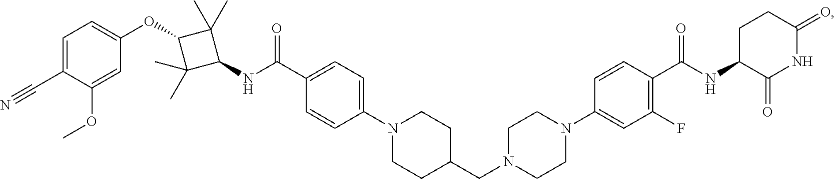 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and salts and solid forms thereof.

In some aspects, the present disclosure is directed to a free base form of Compound A.

In some embodiments, Compound A is crystalline.

In some embodiments, Compound A is amorphous.

In some embodiments, the solid form of Compound A is a crystalline polymorphic form characterized by two or three XRPD signals selected from the group consisting of 18.6 °2θ, 13.9 °2θ, and 15.3 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 18.6 °2θ, 13.9 °2θ, and 15.3 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 80 or FIG. 86.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, or forty-one XRPD signals selected from those set forth in Table 1.

In some embodiments, the solid form of Compound A is a crystalline polymorphic form characterized by two or three XRPD signals selected from the group consisting of 14.4 °2θ, 19.1 °2θ, and 15.8 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 14.4 °2θ, 19.1 °2θ, and 15.8 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 87.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, or forty XRPD signals selected from those set forth in Table 2.

In some embodiments, the solid form of Compound A is a crystalline polymorphic form characterized by two or more, or three or more XRPD signals selected from the group consisting of 20.6 °2θ, 16.1 °2θ, 16.3 °2θ, 17.3 °2θ, and 16.8 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 20.6 °2θ, 16.1 °2θ, 16.3 °2θ, 17.3 °2θ, and 16.8 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 88.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, or nineteen XRPD signals selected from those set forth in Table 3.

In some embodiments, the solid form of Compound A is a crystalline polymorphic form characterized by two or three XRPD signals selected from the group consisting of 14.6 °2θ, 17.7 °2θ, and 16.7 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 14.6 °2θ, 17.7 °2θ, and 16.7 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 89.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four XRPD signals selected from those set forth in Table 4.

In some embodiments, the solid form of Compound A is a crystalline polymorphic form characterized by two or three XRPD signals selected from the group consisting of 14.9 °2θ, 22.6 °2θ, and 7.1 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 14.9 °2θ, 22.6 °2θ, and 7.1 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 90.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, or thirty-five XRPD signals selected from those set forth in Table 5.

In some embodiments, the solid form of Compound A is a crystalline polymorphic form characterized by two or three XRPD signals selected from the group consisting of 3.5 °2θ, 3.6 °2θ, and 15.7 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 3.5 °2θ, 3.6 °2θ, and 15.7 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 91.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, or thirty-six XRPD signals selected from those set forth in Table 6.

In some embodiments, the solid form of Compound A is a crystalline polymorphic form characterized by two or three XRPD signals selected from the group consisting of 4.8 °2θ, 15.7 °2θ, and 17.9 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 4.8 °2θ, 15.7 °2θ, and 17.9 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 92.

In some embodiments, the solid form of Compound A is a crystalline polymorphic form characterized by one, two, three, four, five, six, seven, eight, nine, ten, eleven, twelve, thirteen, fourteen, fifteen, or sixteen XRPD signals selected from those set forth in Table 7.

In some embodiments, the solid form of Compound A is a crystalline polymorphic form characterized by two or three XRPD signals selected from the group consisting of 4.9 °2θ, 15.9 °2θ, and 18.2 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 4.9 °2θ, 15.9 °2θ, and 18.2 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 93.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, or twenty-two XRPD signals selected from those set forth in Table 8.

In some embodiments, the solid form of Compound A is a crystalline polymorphic form characterized by two or three XRPD signals selected from the group consisting of 17.2 °2θ, 21.0 °2θ, and 24.2 °2θ (±0.2 °2θ; ±0.1 °2θ; or ±0.0 °2θ; Cu Kα1 radiation). In some embodiments, the solid form of Compound A is a crystalline polymorphic form characterized by XRPD signals at 17.2 °2θ, 21.0 °2θ, and 24.2 °2θ (±0.2 °2θ; ±0.1 °2θ; or ±0.0 °2θ; Cu Kα1 radiation).

In some embodiments, the solid form of Compound A is a crystalline polymorphic form characterized by an XRPD spectrum substantially similar to that shown in FIG. 94.

In some embodiments, the solid form of Compound A is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, or forty-two XRPD signals selected from those set forth in Table 9.

In some aspects, the present disclosure is directed to a tosylate salt of Compound A.

In some embodiments, the tosylate salt is crystalline.

In some embodiments, the tosylate salt is amorphous.

In some embodiments, the tosylate salt is a crystalline polymorphic form characterized by two or three XRPD signals selected from the group consisting of 3.5 °2θ, 22.0 °2θ, and 23.0 °2θ (±0.2 °2θ; ±0.1 °2θ; or ±0.0 °2θ; Cu Kα1 radiation). In some embodiments, the tosylate salt is a crystalline polymorphic form characterized by XRPD signals at 3.5 °2θ, 22.0 °2θ, and 23.0 °2θ (±0.2 °2θ; ±0.1 °2θ; or ±0.0 °2θ; Cu Kα1 radiation).

In some embodiments, the tosylate salt is a crystalline polymorphic form characterized by an XRPD spectrum substantially similar to that shown in FIG. 95.

In some embodiments, the tosylate salt is a crystalline polymorphic form characterized by one, two, three, four, five, six, seven, eight, nine, ten, eleven, twelve, or thirteen XRPD signals selected from those set forth in Table 10.

In some embodiments, the tosylate salt is a 1:1 tosylate:Compound A salt.

In some embodiments, the tosylate salt is a 2:1 tosylate:Compound A salt.

In some embodiments, the tosylate salt is a 1:2 tosylate:Compound A salt.

In some aspects, the present disclosure is directed to a phosphate salt of Compound A.

In some embodiments, the phosphate salt is crystalline.

In some embodiments, the phosphate salt is amorphous.

In some embodiments, the phosphate salt is a crystalline polymorphic form characterized by two or three XRPD signals selected from the group consisting of 23.6 °2θ, 3.3 °2θ, and 19.9 °2θ (±0.2 °2θ; ±0.1 °2θ; or ±0.0 °2θ; Cu Kα1 radiation). In some embodiments, the phosphate salt is a crystalline polymorphic form characterized by XRPD signals at 23.6 °2θ, 3.3 °2θ, and 19.9 °2θ (±0.2 °2θ; ±0.1 °2θ; or ±0.0 °2θ; Cu Kα1 radiation).

In some embodiments, the phosphate salt is a crystalline polymorphic form characterized by an XRPD spectrum substantially similar to that shown in FIG. 96.

In some embodiments, the phosphate salt is a crystalline polymorphic form characterized by one, two, three, four, five, six, seven, eight, nine, ten, eleven, twelve, thirteen, or fourteen XRPD signals selected from those set forth in Table 11.

In some embodiments, the phosphate salt is a 1:1 phosphate:Compound A salt, i.e., a monophosphate salt of Compound A.

In some embodiments, the phosphate salt is a 2:1 phosphate:Compound A salt, i.e., a bisphosphate salt of Compound A.

In some embodiments, the phosphate salt is a 1:1 phosphate:Compound A salt or a 2:1 phosphate:Compound A salt.

In some embodiments, the phosphate salt is a 1:2 phosphate:Compound A salt, i.e., a hemiphosphate salt of Compound A.

In some aspects, the present disclosure is directed to a besylate salt of Compound A.

In some embodiments, the besylate salt is crystalline.

In some embodiments, the besylate salt is amorphous.

In some embodiments, the besylate salt is a crystalline polymorphic form characterized by two or three XRPD signals selected from the group consisting of 18.5 °2θ, 18.3 °2θ, and 22.6 °2θ (±0.2 °2θ; ±0.1 °2θ; or ±0.0 °2θ; Cu Kα1 radiation). In some embodiments, the besylate salt is a crystalline polymorphic form characterized by XRPD signals at 18.5 °2θ, 18.3 °2θ, and 22.6 °2θ (±0.2 °2θ; ±0.1 °2θ; or ±0.0 °2θ; Cu Kα1 radiation).

In some embodiments, the besylate salt is a crystalline polymorphic form characterized by an XRPD spectrum substantially similar to that shown in FIG. 97.

In some embodiments, the besylate salt is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty XRPD signals selected from those set forth in Table 12.

In some embodiments, the besylate salt is a 1:1 besylate:Compound A salt.

In some embodiments, the besylate salt is a 2:1 besylate:Compound A salt.

In some embodiments, the besylate salt is a 1:2 besylate:Compound A salt.

In some aspects, the present disclosure is directed to a chloride salt of Compound A.

In some embodiments, the chloride salt is a 1:1 chloride:Compound A salt.

In some embodiments, the chloride salt is a 2:1 chloride:Compound A salt.

In some embodiments, the chloride salt is a 1:1 chloride:Compound A salt or a 2:1 chloride:Compound A salt.

In some embodiments, the chloride salt is a 1:2 chloride:Compound A salt.

In some aspects, the present disclosure is directed to a method of treating prostate cancer in a subject need thereof comprising administering to the subject a therapeutically effective amount of a solid form or salt of Compound A disclosed herein.

In some embodiments, the method further comprises administering an effective amount of at least one additional anti-cancer agent to the subject.

In some embodiments, the prostate cancer is metastatic prostate cancer.

In some embodiments, the prostate cancer is castrate-resistant prostate cancer.

In some embodiments, the prostate cancer is metastatic castrate-resistant prostate cancer.

In some embodiments, the prostate cancer is castrate-sensitive prostate cancer.

In some embodiments, the prostate cancer is metastatic castrate-sensitive prostate cancer.

In some embodiments, the prostate cancer has not been previously treated with a second generation antiandrogen. In some embodiments, the prostate cancer has not been previously treated with an androgen biosynthesis inhibitor or an androgen receptor blocker. In some embodiments, the prostate cancer has not been previously treated with an androgen biosynthesis inhibitor. In some embodiments, the prostate cancer has not been previously treated with an androgen receptor blocker. In some embodiments, the prostate cancer has not been previously treated with abiraterone acetate. In some embodiments, the prostate cancer has not been previously treated with an androgen receptor blocker selected from enzalutamide, darolutamide, and apalutamide. In some embodiments, the subject has not been previously administered a second generation antiandrogen. In some embodiments, the subject has not been previously administered an androgen biosynthesis inhibitor or an androgen receptor blocker. In some embodiments, the subject has not been previously administered an androgen biosynthesis inhibitor. In some embodiments, the subject has not been previously administered an androgen receptor blocker. In some embodiments, the subject has not been previously administered abiraterone acetate. In some embodiments, the subject has not been previously administered an androgen receptor blocker selected from enzalutamide, darolutamide, and apalutamide.

In some embodiments, the prostate cancer is prostate cancer naïve to novel hormonal agents (NHA). In some embodiments, the prostate cancer naïve to novel hormonal agents (NHA) has not been previously treated with one or more second generation antiandrogens. In some embodiments, the prostate cancer naïve to novel hormonal agents (NHA) has not been previously treated with an androgen biosynthesis inhibitor or an androgen receptor blocker. In some embodiments, the prostate cancer naïve to novel hormonal agents (NHA) has not been previously treated with an androgen biosynthesis inhibitor. In some embodiments, the prostate cancer naïve to novel hormonal agents (NHA) has not been previously treated with an androgen receptor blocker. In some embodiments, the prostate cancer naïve to novel hormonal agents (NHA) has not been previously treated with abiraterone acetate. In some embodiments, the prostate cancer naïve to novel hormonal agents (NHA) has not been previously treated with an androgen receptor blocker selected from enzalutamide, darolutamide, and apalutamide.

In some embodiments, the prostate cancer is metastatic prostate cancer naïve to novel hormonal agents (NHA). In some embodiments, the metastatic prostate cancer naïve to novel hormonal agents (NHA) has not been previously treated with one or more second generation antiandrogens. In some embodiments, the metastatic prostate cancer naïve to novel hormonal agents (NHA) has not been previously treated with an androgen biosynthesis inhibitor or an androgen receptor blocker. In some embodiments, the metastatic prostate cancer naïve to novel hormonal agents (NHA) has not been previously treated with an androgen biosynthesis inhibitor. In some embodiments, the metastatic prostate cancer naïve to novel hormonal agents (NHA) has not been previously treated with an androgen receptor blocker. In some embodiments, the metastatic prostate cancer naïve to novel hormonal agents (NHA) has not been previously treated with abiraterone acetate. In some embodiments, the metastatic prostate cancer naïve to novel hormonal agents (NHA) has not been previously treated with an androgen receptor blocker selected from enzalutamide, darolutamide, and apalutamide.

In some embodiments, the prostate cancer is castrate-resistant prostate cancer naïve to novel hormonal agents (NHA). In some embodiments, the castrate-resistant prostate cancer naïve to novel hormonal agents (NHA) has not been previously treated with one or more second generation antiandrogens. In some embodiments, the castrate-resistant prostate cancer naïve to novel hormonal agents (NHA) has not been previously treated with an androgen biosynthesis inhibitor or an androgen receptor blocker. In some embodiments, the castrate-resistant prostate cancer naïve to novel hormonal agents (NHA) has not been previously treated with an androgen biosynthesis inhibitor. In some embodiments, the castrate-resistant prostate cancer naïve to novel hormonal agents (NHA) has not been previously treated with an androgen receptor blocker. In some embodiments, the castrate-resistant prostate cancer naïve to novel hormonal agents (NHA) has not been previously treated with abiraterone acetate. In some embodiments, the castrate-resistant prostate cancer naïve to novel hormonal agents (NHA) has not been previously treated with an androgen receptor blocker selected from enzalutamide, darolutamide, and apalutamide.

In some embodiments, the prostate cancer is castrate-sensitive prostate cancer naïve to novel hormonal agents (NHA). In some embodiments, the castrate-sensitive prostate cancer naïve to novel hormonal agents (NHA) has not been previously treated with one or more second generation antiandrogens. In some embodiments, the castrate-sensitive prostate cancer naïve to novel hormonal agents (NHA) has not been previously treated with an androgen biosynthesis inhibitor or an androgen receptor blocker. In some embodiments, the castrate-sensitive prostate cancer naïve to novel hormonal agents (NHA) has not been previously treated with an androgen biosynthesis inhibitor. In some embodiments, the castrate-sensitive prostate cancer naïve to novel hormonal agents (NHA) has not been previously treated with an androgen receptor blocker. In some embodiments, the castrate-sensitive prostate cancer naïve to novel hormonal agents (NHA) has not been previously treated with abiraterone acetate. In some embodiments, the castrate-sensitive prostate cancer naïve to novel hormonal agents (NHA) has not been previously treated with an androgen receptor blocker selected from enzalutamide, darolutamide, and apalutamide.

In some embodiments, the prostate cancer is metastatic castrate-resistant prostate cancer naïve to novel hormonal agents (NHA). In some embodiments, the metastatic castrate-resistant prostate cancer naïve to novel hormonal agents (NHA) has not been previously treated with one or more second generation antiandrogens. In some embodiments, the metastatic castrate-resistant prostate cancer naïve to novel hormonal agents (NHA) has not been previously treated with an androgen biosynthesis inhibitor or an androgen receptor blocker. In some embodiments, the metastatic castrate-resistant prostate cancer naïve to novel hormonal agents (NHA) has not been previously treated with an androgen biosynthesis inhibitor. In some embodiments, the metastatic castrate-resistant prostate cancer naïve to novel hormonal agents (NHA) has not been previously treated with an androgen receptor blocker. In some embodiments, the metastatic castrate-resistant prostate cancer naïve to novel hormonal agents (NHA) has not been previously treated with abiraterone acetate. In some embodiments, the metastatic castrate-resistant prostate cancer naïve to novel hormonal agents (NHA) has not been previously treated with an androgen receptor blocker selected from enzalutamide, darolutamide, and apalutamide.

In some embodiments, the prostate cancer is metastatic castrate-sensitive prostate cancer naïve to novel hormonal agents (NHA). In some embodiments, the metastatic castrate-sensitive prostate cancer naïve to novel hormonal agents (NHA) has not been previously treated with one or more second generation antiandrogens. In some embodiments, the metastatic castrate-sensitive prostate cancer naïve to novel hormonal agents (NHA) has not been previously treated with an androgen biosynthesis inhibitor or an androgen receptor blocker. In some embodiments, the metastatic castrate-sensitive prostate cancer naïve to novel hormonal agents (NHA) has not been previously treated with an androgen biosynthesis inhibitor. In some embodiments, the metastatic castrate-sensitive prostate cancer naïve to novel hormonal agents (NHA) has not been previously treated with an androgen receptor blocker. In some embodiments, the metastatic castrate-sensitive prostate cancer naïve to novel hormonal agents (NHA) has not been previously treated with abiraterone acetate. In some embodiments, the metastatic castrate-sensitive prostate cancer naïve to novel hormonal agents (NHA) has not been previously treated with an androgen receptor blocker selected from enzalutamide, darolutamide, and apalutamide.

In some embodiments, the second generation antiandrogen is an androgen biosynthesis inhibitor or an androgen receptor blocker.

In some embodiments, the androgen biosynthesis inhibitor is abiraterone acetate.

In some embodiments, the androgen receptor blocker is selected from enzalutamide, darolutamide, and apalutamide.

Additional features, advantages, and aspects of the present disclosure are set forth or apparent from consideration of the following detailed description, drawings, and claims. Moreover, it is to be understood that both the foregoing summary of the present disclosure and the following detailed description are exemplary and intended to provide further explanation without limiting the scope of the present disclosure as claimed.

### BRIEF DESCRIPTION OF THE DRAWINGS

The accompanying drawings, which are included to provide a further understanding of the present disclosure, are incorporated in and constitute a part of this specification, illustrate aspects of the present disclosure and, together with the detailed description, serve to explain the principles of the present disclosure. The patent or application file contains at least one drawing executed in color. Copies of this patent or patent application publication with color drawing(s) will be provided by the Office upon request and payment of the necessary fee.

FIG. 1 depicts an XRPD pattern of Compound A.

FIG. 2 depicts an ¹H-NMR spectrum of Compound A, recorded in DMSO-d₆.

FIG. 3 depicts an ¹⁹F-NMR spectrum of Compound A, recorded in DMSO-d₆.

FIG. 4 depicts an TG/DSC thermogram of Compound A.

FIG. 5 depicts an DSC thermogram of Compound A (first heating cycle).

FIG. 6 depicts an DSC thermogram of Compound A (first cooling cycle).

FIG. 7 depicts an DSC thermogram of Compound A (second heating cycle).

FIG. 8 depicts an DSC thermogram of Compound A (second cooling cycle).

FIG. 9 depicts an VT-XRPD patterns of Compound A.

FIG. 10 depicts an DVS isotherm plot of Compound A.

FIG. 11 depicts an DVS kinetic plot of Compound A.

FIG. 12 depicts an overlay of the XRPD pattern of Compound A (top) and the XRPD pattern of Compound A post DVS analysis (bottom).

FIG. 13 depicts an TG/DSC thermogram of Compound A.

FIG. 14 depicts an FT-IR spectrum of potential chloride Pattern 2 from DCM:methanol (50:50% v/v).

FIG. 15 depicts an TG/DSC thermogram of potential chloride Pattern 2 from DCM.

FIG. 16 depicts an TG/DSC thermogram of potential chloride Pattern 2 from DCM:methanol (50:50% v/v).

FIG. 17 depicts an FT-IR spectrum of tosylate Pattern 1 from THF.

FIG. 18 depicts an TG/DSC thermogram of tosylate Pattern 1 from THF.

FIG. 19 depicts an ¹H-NMR spectrum of tosylate Pattern 1 from THF, recorded in DMSO-d₆.

FIG. 20 depicts an FT-IR spectrum of besylate Pattern 1 from THF:water (98:2% v/v).

FIG. 21 depicts an TG/DSC thermogram of besylate Pattern 1 from THF:water (98:2% v/v).

FIG. 22 depicts an ¹H-NMR spectrum of besylate Pattern 1 from THF:water (98:2% v/v), recorded in DMSO-d₆.

FIG. 23 depicts an FT-IR spectrum of phosphate Pattern 1 from THF:water (98:2% v/v).

FIG. 24 depicts an TG/DSC thermogram of phosphate Pattern 1 from THF:water (98:2% v/v).

FIG. 25 depicts an ¹H-NMR spectrum of phosphate Pattern 1 from THF:water (98:2% v/v), recorded in DMSO-d₆.

FIG. 26 depicts an ³¹P-NMR spectrum of phosphate Pattern 1 from THF:water (98:2% v/v), recorded in DMSO-d₆.

FIG. 27 depicts an TG/DSC thermogram of the amorphous chloride.

FIG. 28 depicts an DSC thermogram of the amorphous chloride (first heating cycle).

FIG. 29 depicts an DSC thermogram of the amorphous chloride (cooling cycle).

FIG. 30 depicts an DSC thermogram of the amorphous chloride (second heating cycle).

FIG. 31 depicts an additional DSC thermogram of the amorphous chloride (first heating cycle).

FIG. 32 depicts an additional DSC thermogram of the amorphous chloride (cooling cycle).

FIG. 33 depicts an additional DSC thermogram of the amorphous chloride (second heating cycle).

FIG. 34 depicts an overlay of XRPD patterns of free base Pattern 9 from 1,4-dioxane/L-Histidine (top), free base Pattern 4 post-storage at 40° C./75% RH (middle), and free base Pattern 4 (bottom).

FIG. 35 depicts an TG/DSC thermogram of free base Pattern 9.

FIG. 36 depicts an overlay of the XRPD patterns of the acetonitrile and anisole crystallization screen samples pre- and post-temperature cycling.

FIG. 37 depicts an overlay of the XRPD patterns of the ethyl formate and isopropyl acetate crystallization screen samples pre- and post-temperature cycling.

FIG. 38 depicts an overlay of the XRPD patterns of the THF and toluene crystallization screen samples pre- and post-temperature cycling.

FIG. 39 depicts an TG/DSC thermogram of free base Pattern 1 from 2-ethoxyethanol.

FIG. 40 depicts an FT-IR spectrum of free base Pattern 1 from THF.

FIG. 41 depicts an TG/DSC thermogram of free base Pattern 2 from DCM:methanol (50:50% v/v).

FIG. 42 depicts an FT-IR spectrum of free base Pattern 2 from DCM:methanol (50:50% v/v).

FIG. 43 depicts an TG/DSC thermogram of free base Pattern 3 from 1,4-dioxane and succinic acid.

FIG. 44 depicts an FT-IR spectrum of free base Pattern 3 from 1,4-dioxane and hydrochloride acid.

FIG. 45 depicts an TG/DSC thermogram of free base Pattern 4 from 1,4-dioxane and malonic acid.

FIG. 46 depicts an FT-IR spectrum of free base Pattern 4 from 1,4-dioxane and benzoic acid.

FIG. 47 depicts an TG/DSC thermogram of free base Pattern 6 from DCM post temperature cycling.

FIG. 48 depicts an FT-IR spectrum of free base Pattern 6 from DCM post temperature cycling.

FIG. 49 depicts an TG/DSC thermogram of free base Pattern 8 from anisole.

FIG. 50 depicts a TG/DSC thermogram of free base Pattern 8 from anisole (dried further).

FIG. 51 depicts a TG/DSC thermogram of free base Pattern 8 from toluene.

FIG. 52 depicts an FT-IR spectrum of free base Pattern 8 from anisole.

FIG. 53 depicts an FT-IR spectrum of free base Pattern 8 from toluene.

FIG. 54 depicts a TG/DSC thermogram of free base Pattern 1.

FIG. 55 depicts an ¹H-NMR spectrum of free base Pattern 1, recorded in DMSO-d₆.

FIG. 56 depicts a DSC thermogram of free base Pattern 1 (first heating cycle).

FIG. 57 depicts a DSC thermogram of free base Pattern 1 (cooling cycle).

FIG. 58 depicts a DSC thermogram of free base Pattern 1 (second heating cycle).

FIG. 59 depicts a modulated DSC thermogram of the 3-gram batch of free base Pattern 1.

FIG. 60 depicts a DVS isotherm plot of free base Pattern 1.

FIG. 61 depicts a DVS kinetic plot of free base Pattern 1.

FIG. 62 depicts an overlay of the XRPD pattern of free base Pattern 1 (top) and the XRPD pattern of free base Pattern 1 post DVS analysis (bottom).

FIG. 63 depicts a TG/DSC thermogram of free base Pattern 2.

FIG. 64 depicts an ¹H-NMR spectrum of free base Pattern 2, recorded in DMSO-d₆.

FIG. 65 depicts a TG/DSC thermogram of free base Pattern 6.

FIG. 66 depicts an ¹H-NMR spectrum of free base Pattern 6, recorded in DMSO-d₆.

FIG. 67 depicts an ¹H-NMR spectrum of free base Pattern 1 after heating to 250° C., recorded in DMSO-d₆.

FIG. 68 depicts an ¹H-NMR spectrum of tosylate Pattern 1, recorded in DMSO-d₆.

FIG. 69 depicts a TG/DSC thermogram of tosylate Pattern 1.

FIG. 70 depicts an ¹H-NMR spectrum of besylate Pattern 1, recorded in DMSO-d₆.

FIG. 71 depicts a TG/DSC thermogram of besylate Pattern 1.

FIG. 72 depicts an ¹H-NMR spectrum of phosphate Pattern 1, recorded in DMSO-d₆.

FIG. 73 depicts a ³¹P-NMR spectrum of phosphate Pattern 1, recorded in DMSO-d₆.

FIG. 74 depicts a TG/DSC thermogram of phosphate Pattern 1.

FIG. 75 depicts an overlay of the XRPD patterns of free base Pattern 1 post stability analysis.

FIG. 76 depicts an overlay of the XRPD patterns of free base Pattern 2 post stability analysis.

FIG. 77 depicts an overlay of the XRPD patterns of tosylate Pattern 1 post stability analysis.

FIG. 78 depicts an overlay of the XRPD patterns of besylate Pattern 1 post stability analysis.

FIG. 79 depicts an overlay of the XRPD patterns of the scale up attempts from ethanol.

FIG. 80 depicts an XRPD pattern of the scaled-up batch of free base Pattern 1.

FIG. 81 depicts an ¹H-NMR spectrum of the 3-gram batch of free base Pattern 1, recorded in DMSO-d₆.

FIG. 82 depicts a TG/DSC thermogram of 3-gram batch of free base Pattern 1.

FIG. 83 depicts a DSC thermogram of 3-gram batch of free base Pattern 1 (first heating cycle).

FIG. 84 depicts a DSC thermogram of 3-gram batch of free base Pattern 1 (cooling cycle).

FIG. 85 depicts a DSC thermogram of 3-gram batch of free base Pattern 1 (second heating cycle).

FIG. 86 depicts an XRPD pattern of free base Pattern 1.

FIG. 87 depicts an XRPD pattern of free base Pattern 2.

FIG. 88 depicts an XRPD pattern of free base Pattern 3.

FIG. 89 depicts an XRPD pattern of free base Pattern 4.

FIG. 90 depicts an XRPD pattern of free base Pattern 5.

FIG. 91 depicts an XRPD pattern of free base Pattern 6.

FIG. 92 depicts an XRPD pattern of free base Pattern 7.

FIG. 93 depicts an XRPD pattern of free base Pattern 8.

FIG. 94 depicts an XRPD pattern of free base Pattern 9.

FIG. 95 depicts an XRPD pattern of tosylate Pattern 1.

FIG. 96 depicts an XRPD pattern of phosphate Pattern 1.

FIG. 97 depicts an XRPD pattern of besylate Pattern 1.

### DETAILED DESCRIPTION

The present disclosure provides salts and polymorphic salt forms of Compound A that are useful in the preparation of a medicament and/or as pharmaceutical agents. In some embodiments, one or more of the salts and/or salt forms described herein can be formulated into a pharmaceutical composition.

### Definitions

Compound A of the present disclosure refers to 4-(4-((1-(4-(((1R,3R)-3-(4-cyano-3-methoxyphenoxy)-2,2,4,4-tetramethylcyclobutyl) carbamoyl)phenyl) piperidin-4-yl)methyl) piperazin-1-yl)-N—((S)-2,6-dioxopiperidin-3-yl)-2-fluorobenzamide, which has the following structure:

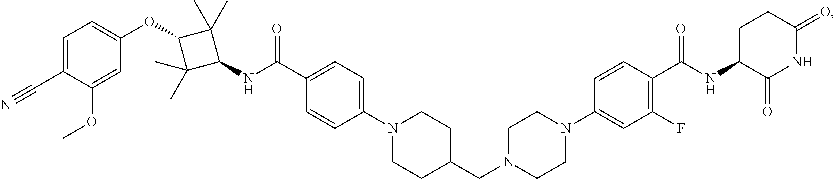 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Compound A can be prepared as described in US Patent Application Publication No. 2021/0196710 A1, which is incorporated herein by reference.

The terms “powder X-ray diffraction pattern”, “PXRD pattern”, “X-ray powder diffraction pattern”, and “XRPD pattern” are used interchangeably and refer to the experimentally observed diffractogram or parameters derived therefrom. Powder X-ray diffraction patterns are typically characterized by peak position (abscissa) and peak intensities (ordinate). The term “peak intensities” refers to relative signal intensities within a given X-ray diffraction pattern. Factors which can affect the relative peak intensities are sample thickness and preferred orientation (i.e., the crystalline particles are not distributed randomly). The term “peak positions” as used herein refers to X-ray reflection positions as measured and observed in powder X-ray diffraction experiments. Peak positions are directly related to the dimensions of the unit cell. The peaks, identified by their respective peak positions, are extracted from the diffraction patterns for the various polymorphic forms of salts of Compound A.

The terms “2 theta value”, “2θ”, “2 θ”, “°2θ”, or “°2 θ” refer to the peak position in degrees based on the experimental setup of the X-ray diffraction experiment and is a common abscissa unit in diffraction patterns. In general, the experimental setup requires that if a reflection is diffracted when the incoming beam forms an angle theta (θ) with a certain lattice plane, the reflected beam is recorded at an angle 2 theta (2 θ). It should be understood that reference herein to specific 2θ values for a specific polymorphic form is intended to mean the 2θ values (in degrees) as measured using the X-ray diffraction experimental conditions as described herein.

“Preferred orientation effects” refer to variable peak intensities or relative intensity differences between different PXRD measurements of the same samples that can be due to the orientation of the particles. Without wishing to be bound by theory, in PXRD it can be desirable to have a sample in which particles are oriented randomly (e.g., a powder). However, it can be difficult or in some cases impossible to achieve truly random particle orientations in practice. As particle size increases, the randomness of particle orientation can decrease, leading to increased challenges with achieving a preferred orientation. Without wishing to be bound by theory, a smaller particle size can reduce technical challenges associated with preferred orientation and allow for more accurate representation of peaks. However, one of skill in the art will understand how to reduce or mitigate preferred orientation effects and will recognize preferred orientation effects that can exist even between two different measurements of the same sample. For instance, in some embodiments, differences in resolution or relative peak intensities can be attributed to preferred orientation effects.

As used herein, the term “substantially pure” with reference to a particular salt (or to a mixture of two or more salts) of a compound indicates the salt (or a mixture) includes less than 10%, less than 5%, less than 3%, less than 1%, less than 0.5%, less than 0.2%, or less than 0.1% by weight of impurities, including other salt forms of the compound. Such purity may be determined, for example, by powder X-ray diffraction.

As used herein, the term “polymorph” or “salt form” refers to different crystalline forms of the same compound and other solid state molecular forms including pseudo-polymorphs, such as hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such as the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way (S. Byrn et al, Pharmaceutical Solids: A Strategic Approach to Regulatory Considerations, Pharmaceutical research, Vol. 12, No. 7, p. 945-954, 1995; J. K. Haleblian and W. McCrone, Pharmaceutical Applications of Polymorphism, Journal of Pharmaceutical Sciences, Vol. 58, No. 8, p. 91 1-929, 1969).

Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

The term “amorphous” refers to any solid substance which (i) lacks order in three dimensions, or (ii) exhibits order in less than three dimensions, order only over short distances (e.g., less than 10 A), or both. Thus, amorphous substances include partially crystalline materials and crystalline mesophases with, e.g., one- or two-dimensional translational order (liquid crystals), orientational disorder (orientationally disordered crystals), or conformational disorder (conformationally disordered crystals). Amorphous solids may be characterized by known techniques, including powder X-ray diffraction (PXRD) crystallography, solid state nuclear magnet resonance (ssNMR) spectroscopy, differential scanning calorimetry (DSC), or some combination of these techniques. Amorphous solids give diffuse PXRD patterns, typically comprised of one or two broad peaks (i.e., peaks having base widths of about 5 °2θ or greater).

The term “crystalline” refers to any solid substance exhibiting three-dimensional order, which, in contrast to an amorphous solid substance, gives a distinctive PXRD pattern with sharply defined peaks.

The term “ambient temperature” refers to a temperature condition typically encountered in a laboratory setting. This includes the approximate temperature range of about 20 to about 30° C.

The term “detectable amount” refers to an amount or amount per unit volume that can be detected using conventional techniques, such as X-ray powder diffraction, differential scanning calorimetry, HPLC, Fourier Transform Infrared Spectroscopy (FT-IR), Raman spectroscopy, and the like.

The term “solvate” describes a molecular complex comprising the drug substance and a stoichiometric or non-stoichiometric amount of one or more solvent molecules (e.g., ethanol). When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex may be non-stoichiometric.

The term “hydrate” describes a solvate comprising the drug substance and a stoichiometric or non-stoichiometric amount of water.

The term “relative humidity” refers to the ratio of the amount of water vapor in air at a given temperature to the maximum amount of water vapor that can be held at that temperature and pressure, expressed as a percentage.

The term “relative intensity” refers to an intensity value derived from a sample X-ray diffraction pattern. The complete ordinate range scale for a diffraction pattern is assigned a value of 100. A peak having intensity falling between about 50% to about 100% on this scale intensity is termed very strong (vs); a peak having intensity falling between about 50% to about 25% is termed strong(s). Additional weaker peaks are present in typical diffraction patterns and are also characteristic of a given polymorph, wherein the additional peaks are termed medium (m), weak (w) and very weak (vw).

The term “slurry” refers to a solid substance suspended in a liquid medium, typically water or an organic solvent.

The term “under vacuum” refers to typical pressures obtainable by a laboratory oil or oil-free diaphragm vacuum pump.

The term “pharmaceutical composition” refers to a composition comprising one or more of the polymorphic forms of salts of Compound A described herein, and other chemical components, such as physiologically/pharmaceutically acceptable carriers, diluents, vehicles and/or excipients. The purpose of a pharmaceutical composition is to facilitate administration of a compound to an organism, such as a human or other mammals.

The term “pharmaceutically acceptable” “carrier”, “diluent”, “vehicle”, or “excipient” refers to a material (or materials) that may be included with a particular pharmaceutical agent to form a pharmaceutical composition, and may be solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like.

Similarly, the carrier or diluent may include time-delay or time-release material known in the art, such as glyceryl monostearate or glyceryl distearate alone or with a wax, ethylcellulose, hydroxypropyl methylcellulose, methylmethacrylate and the like.

The term “treating”, as used herein, unless otherwise indicated, means reversing, alleviating, or inhibiting the progress of the disorder or condition to which such term applies, or one or more symptoms of such disorder or condition. The term “treatment”, as used herein, unless otherwise indicated, refers to the act of “treating” as defined immediately above. For example, the terms “treat”, “treating” and “treatment” can refer to a method of alleviating or abrogating a particular disorder and/or one or more of its attendant symptoms.

As used herein, “subject” means a human or animal (in the case of an animal, the subject can be a mammal). In one aspect, the subject is a human. In one aspect, the subject is a male.

Prostate cancer is the uncontrolled growth of cancerous cells in the prostate gland. In some embodiments, the prostate cancer is metastatic prostate cancer, castrate-resistant prostate cancer, metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), metastatic castrate-resistant prostate cancer naïve to novel hormonal agents (NHA), castrate-sensitive prostate cancer naïve to novel hormonal agents (NHA), or metastatic castrate-sensitive prostate cancer naïve to novel hormonal agents (NHA).

Metastatic prostate cancer, or metastases, refers to prostate cancer that has spread beyond the prostate to other parts of the body, e.g., bones, lymph nodes, liver, lungs, brain.

Castrate-resistant prostate cancer or castration-resistant prostate cancer (or prostate cancer that is castrate- or castration-resistant) is a type of prostate cancer that keeps growing even when the amount of testosterone in the body is reduced to very low levels.

Metastatic castrate-resistant prostate cancer is a type of prostate cancer that has metastasized and continues to grow even when the amount of testosterone in the body is reduced to very low levels.

Castrate-sensitive prostate cancer or castration-sensitive prostate cancer (CSPC), or prostate cancer that is castrate- or castration-sensitive, is prostate cancer that can be controlled by reducing the amount of androgens (male hormones) in the body (e.g., through castration) and/or prostate cancer that requires androgens to grow and stops growing when androgens are not present. CSPC is also referred to as androgen-dependent prostate cancer, androgen-sensitive prostate cancer, or hormone-sensitive prostate cancer (HSPC).

Metastatic castrate-sensitive prostate cancer is a type of castrate-sensitive prostate cancer that has metastasized and requires androgens to grow and stops growing when androgens are not present, or can be controlled by reducing the amount of androgens in the body (e.g., through castration).

Prostate cancer naïve to novel hormonal agents (NHA) is prostate cancer that has not been previously treated with one or more second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Metastatic prostate cancer naïve to novel hormonal agents (NHA) is metastatic prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Castrate-resistant prostate cancer naïve to novel hormonal agents (NHA) is castrate-resistant prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Castrate-sensitive prostate cancer naïve to novel hormonal agents (NHA) is castrate-sensitive prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Metastatic castrate-resistant prostate cancer naïve to novel hormonal agents (NHA) is metastatic castrate-resistant prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

Metastatic castrate-sensitive prostate cancer naïve to novel hormonal agents (NHA) is metastatic castrate-sensitive prostate cancer that has not been previously treated with second generation antiandrogens such as androgen biosynthesis inhibitors or androgen receptor blockers. In some embodiments, the androgen biosynthesis inhibitor is abiraterone (e.g., abiraterone acetate). In some embodiments, the androgen receptor blocker is enzalutamide, darolutamide, or apalutamide.

As used herein, the term “anti-cancer agent” is used to describe an anti-cancer agent, or a therapeutic agent administered concurrently with an anti-cancer agent (e.g., palonosetron), with which may be co-administered and/or co-formulated with a compound of the disclosure to treat cancer, and the side effects associated with the cancer treatment. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a CDK inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, and mixtures thereof. In one embodiment, the anti-cancer agent is selected from the group consisting of abiraterone, estramustine, docetaxel, ketoconazole, goserelin, histrelin, triptorelin, buserelin, cyproterone, flutamide, bicalutamide, nilutamide, pamidronate, and zolendronate. In one embodiment, the anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, focal adhesion kinase inhibitor, Map kinase inhibitor, VEGF trap antibody, and chemical castration agent.

In some embodiments, the anti-cancer agent is selected from the group consisting of temozolomide, capecitabine, irinotecan, tamoxifen, anastrazole, exemestane, letrozole, DES, Estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

The term “about” is used herein to mean approximately, in the region of, roughly or around. When the term “about” is used in conjunction with a numerical range, it modifies that range by extending the boundaries above and below the numerical values set forth. In general, the term “about” is used herein to modify a numerical value above and below the stated value by a variance of 20%, a variance of 10%, a variance of 5%, a variance of 3%, or a variance of 1%. When used in the context of XRPD peak values, the term “about” can indicate a peak value ±0.20, ±0.15, ±0.10, ±0.05, or ±0.01 °2θ. In some embodiments, when used in the context of XRPD peak values “about” can indicate a peak value at substantially exactly the disclosed peak value.

### Crystalline Forms of Compound A

As set forth below, Compound A can form salts with different acids. In some embodiments, the salts of Compound A described herein exist in various crystalline forms. All PXRD peaks described herein are in ° 2θ (±0.2 °2θ; ±0.1 °2θ; or ±0.0 °2θ; Cu Kα1 radiation). Additionally, all PXRD spectra are obtained using Cu Kα1 X-rays at a wavelength of 1.5406 Å.

### Compound A Free Base Pattern 1

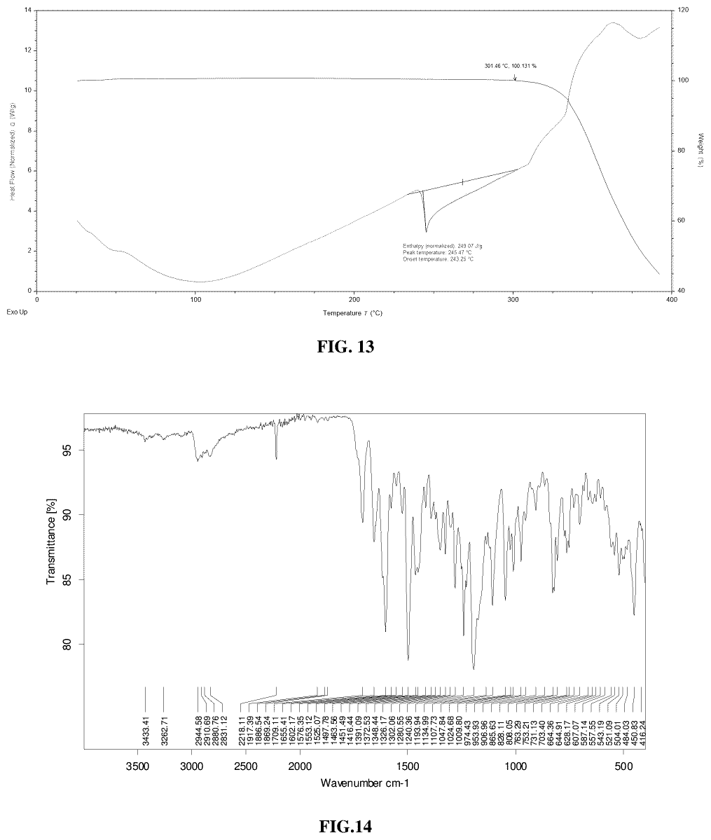

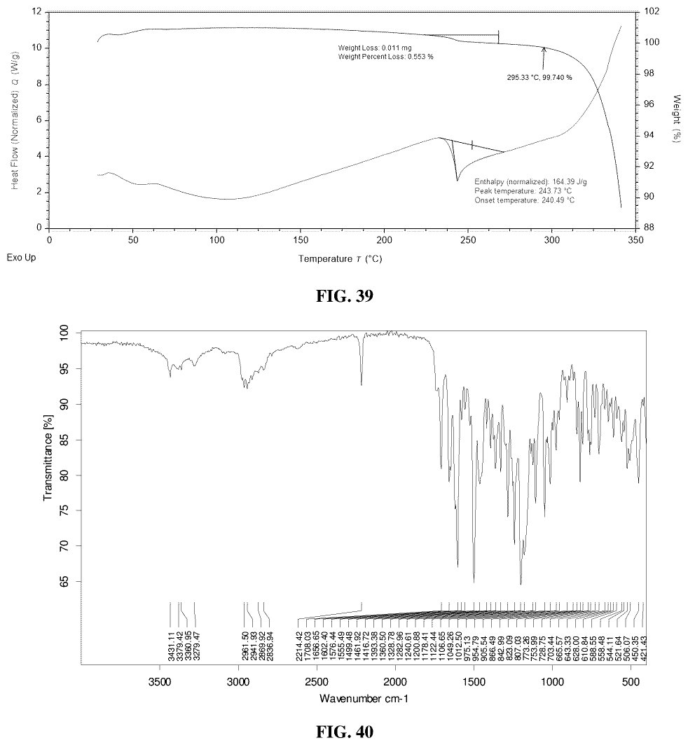

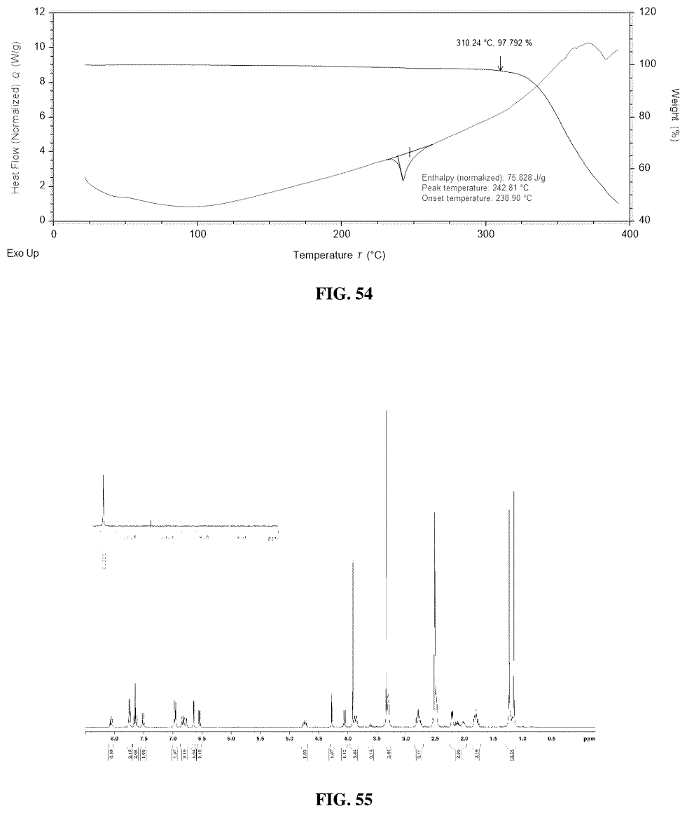

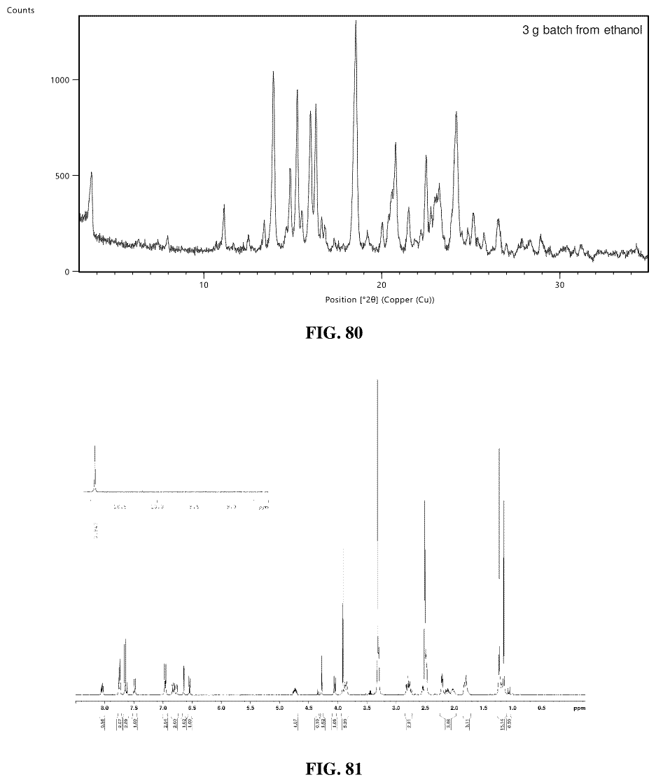

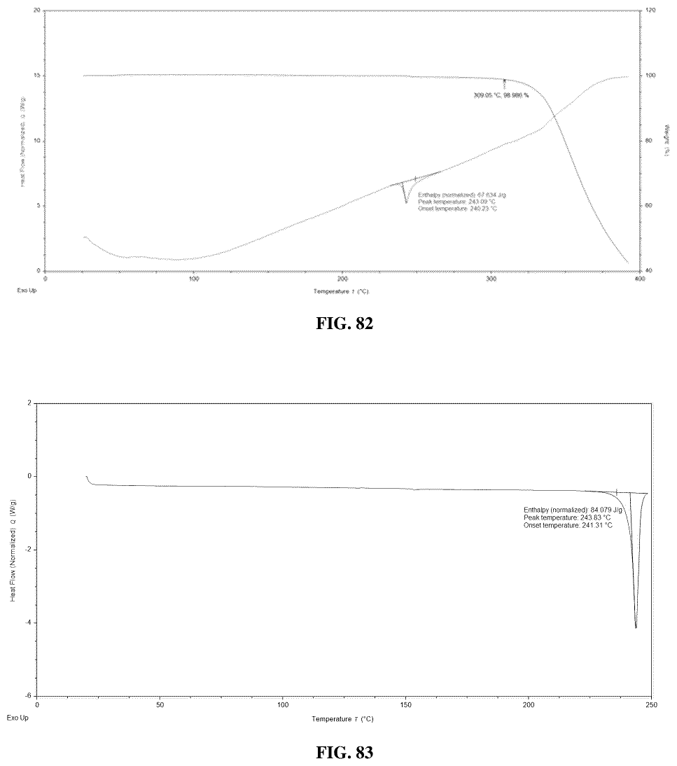

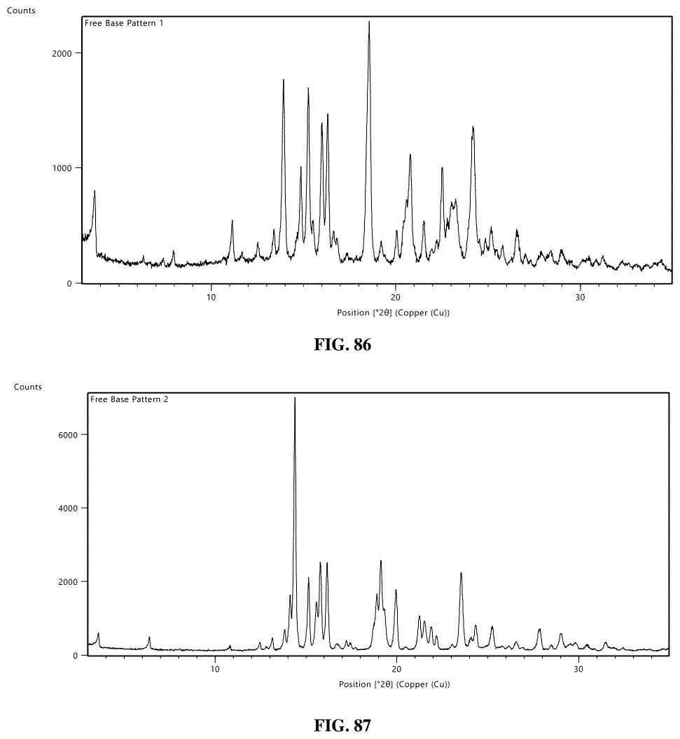

In some embodiments, the present disclosure provides solid forms of Compound A, e.g., crystalline forms of Compound A free base Pattern 1. In some embodiments, the Compound A free base Pattern 1 XRPD profile is substantially similar to that shown in FIG. 80 or FIG. 86. In some embodiments, the Compound A free base Pattern 1 FT-IR spectrum is substantially similar to that shown in FIG. 40. In some embodiments, the Compound A free base Pattern 1 ¹H NMR spectrum is substantially similar to that shown in FIG. 55 or FIG. 81. In some embodiments, the Compound A free base Pattern 1 TGA profile is substantially similar to that shown in FIG. 13, FIG. 39, FIG. 54, or FIG. 82. In some embodiments, the Compound A free base Pattern 1 DSC profile is substantially similar to that shown in FIG. 13, FIG. 39, FIG. 54, or FIG. 82.

In some embodiments, the solid form of Compound A free base Pattern 1 is crystalline Compound A free base Pattern 1 characterized by two or three XRPD signals selected from the group consisting of 18.6 °2θ, 13.9 °2θ, and 15.3 °2θ (±0.2 °2θ; ±0.1 °2θ; or ±0.0 °2θ; Cu Kα1 radiation). In some embodiments, the solid form of Compound A free base Pattern 1 is crystalline Compound A free base Pattern 1 characterized by XRPD signals at 18.6 °2θ, 13.9 °2θ, and 15.3 °2θ (±0.2 °2θ; ±0.1 °2θ; or ±0.0 °2θ; Cu Kα1 radiation).

In some embodiments, the solid form of Compound A free base Pattern 1 is crystalline Compound A free base Pattern 1 characterized by two or more, or three or more XRPD signals selected from the group consisting of 18.6 °2θ, 13.9 °2θ, 15.3 °2θ, 16.3 °2θ, and 16.0 °2θ (±0.2 °2θ; ±0.1 °2θ; or ±0.0 °2θ; Cu Kα1 radiation). In some embodiments, the solid form of Compound A free base Pattern 1 is crystalline Compound A free base Pattern 1 characterized by XRPD signals at 18.6 °2θ, 13.9 °2θ, 15.3 °2θ, 16.3 °2θ, and 16.0 °2θ (±0.2 °2θ; ±0.1 °2θ; or ±0.0 °2θ; Cu Kα1 radiation).

In some embodiments, the solid form of Compound A free base Pattern 1 is crystalline Compound A free base Pattern 1 characterized by two or more, or three or more XRPD signals selected from the group consisting of 18.6 °2θ, 13.9 °2θ, 15.3 °2θ, 16.3 °2θ, 16.0 °2θ, 24.2 °2θ, and 20.8 °2θ (±0.2 °2θ; ±0.1 °2θ; or ±0.0 °2θ; Cu Kα1 radiation). In some embodiments, the solid form of Compound A free base Pattern 1 is crystalline Compound A free base Pattern 1 characterized by XRPD signals at 18.6 °2θ, 13.9 °2θ, 15.3 °2θ, 16.3 °2θ, 16.0 °2θ, 24.2 °2θ, and 20.8 °2θ (±0.2 °2θ; ±0.1 °2θ; or ±0.0 °2θ; Cu Kα1 radiation).

In some embodiments, the solid form of Compound A free base Pattern 1 is crystalline Compound A free base Pattern 1 characterized by two or more, or three or more XRPD signals selected from the group consisting of 18.6 °2θ, 13.9 °2θ, 15.3 °2θ, 16.3 °2θ, 16.0 °2θ, 24.2 °2θ, 20.8 °2θ, 22.5 °2θ, 14.9 °2θ, and 3.7 °2θ (±0.2 °2θ; ±0.1 °2θ; or ±0.0 °2θ; Cu Kα1 radiation). In some embodiments, the solid form of Compound A free base Pattern 1 is crystalline Compound A free base Pattern 1 characterized by XRPD signals at 18.6 °2θ, 13.9 °2θ, 15.3 °2θ, 16.3 °2θ, 16.0 °2θ, 24.2 °2θ, 20.8 °2θ, 22.5 °2θ, 14.9 °2θ, and 3.7 °2θ (±0.2 °2θ; ±0.1 °2θ; or ±0.0 °2θ; Cu Kα1 radiation).

In some embodiments, the crystalline Compound A free base Pattern 1 is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, or forty-one XRPD signals selected from those set forth in Table 1.

TABLE 1
XRPD peak table of free base Pattern 1

| | Pos. | d-spacing | Height | Rel. Int. |
|---|---|---|---|---|
| No. | [° 2θ] | [Å] | [cts] | [%] |
| 1 | 3.7000 | 23.88057 | 641.16 | 29.77 |
| 2 | 6.3237 | 13.97714 | 59.09 | 2.74 |
| 3 | 7.3807 | 11.97772 | 44.38 | 2.06 |
| 4 | 7.9467 | 11.12582 | 125.42 | 5.82 |
| 5 | 11.1345 | 7.94666 | 389.66 | 18.10 |
| 6 | 11.6773 | 7.57845 | 93.78 | 4.35 |
| 7 | 12.5277 | 7.06587 | 184.13 | 8.55 |
| 8 | 13.3953 | 6.61013 | 315.10 | 14.63 |
| 9 | 13.9133 | 6.36516 | 1642.97 | 76.30 |
| 10 | 14.8622 | 5.96084 | 872.25 | 40.51 |
| 11 | 15.2763 | 5.80015 | 1511.82 | 70.21 |
| 12 | 15.5103 | 5.71317 | 372.73 | 17.31 |
| 13 | 16.0052 | 5.53762 | 1268.84 | 58.92 |
| 14 | 16.3082 | 5.43540 | 1331.24 | 61.82 |
| 15 | 16.6231 | 5.33315 | 301.68 | 14.01 |
| 16 | 16.8305 | 5.26790 | 229.03 | 10.64 |
| 17 | 17.3267 | 5.11814 | 93.56 | 4.34 |
| 18 | 18.5579 | 4.78127 | 2153.36 | 100 |
| 19 | 19.2152 | 4.61917 | 219.40 | 10.19 |
| 20 | 20.0526 | 4.42812 | 303.49 | 14.09 |
| 21 | 20.4657 | 4.33967 | 404.08 | 18.77 |
| 22 | 20.7944 | 4.27180 | 996.66 | 46.28 |
| 23 | 21.5336 | 4.12680 | 392.13 | 18.21 |
| 24 | 22.2132 | 4.00206 | 226.38 | 10.51 |
| 25 | 22.4984 | 3.95197 | 882.82 | 41.00 |
| 26 | 22.9853 | 3.86934 | 544.07 | 25.27 |
| 27 | 23.2645 | 3.82354 | 578.73 | 26.88 |
| 28 | 24.2381 | 3.67212 | 1186.56 | 55.10 |
| 29 | 24.8457 | 3.58368 | 239.10 | 11.10 |
| 30 | 25.1442 | 3.54180 | 332.80 | 15.46 |
| 31 | 25.7829 | 3.45549 | 185.60 | 8.62 |
| 32 | 26.5158 | 3.36163 | 283.85 | 13.18 |
| 33 | 27.0060 | 3.30172 | 116.95 | 5.43 |
| 34 | 27.9072 | 3.19711 | 131.95 | 6.13 |
| 35 | 28.4031 | 3.14241 | 152.44 | 7.08 |
| 36 | 28.9434 | 3.08496 | 152.29 | 7.07 |
| 37 | 30.4651 | 2.93425 | 92.39 | 4.29 |
| 38 | 30.8466 | 2.89882 | 76.26 | 3.54 |
| 39 | 31.1968 | 2.86708 | 104.15 | 4.84 |
| 40 | 32.2339 | 2.77716 | 63.57 | 2.95 |
| 41 | 34.3944 | 2.60750 | 68.99 | 3.20 |

### Compound A Free Base Pattern 2

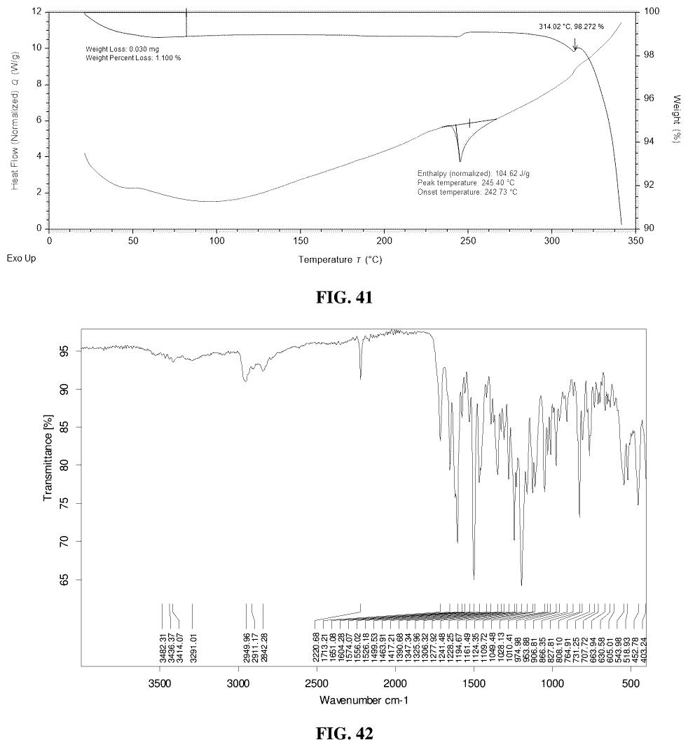

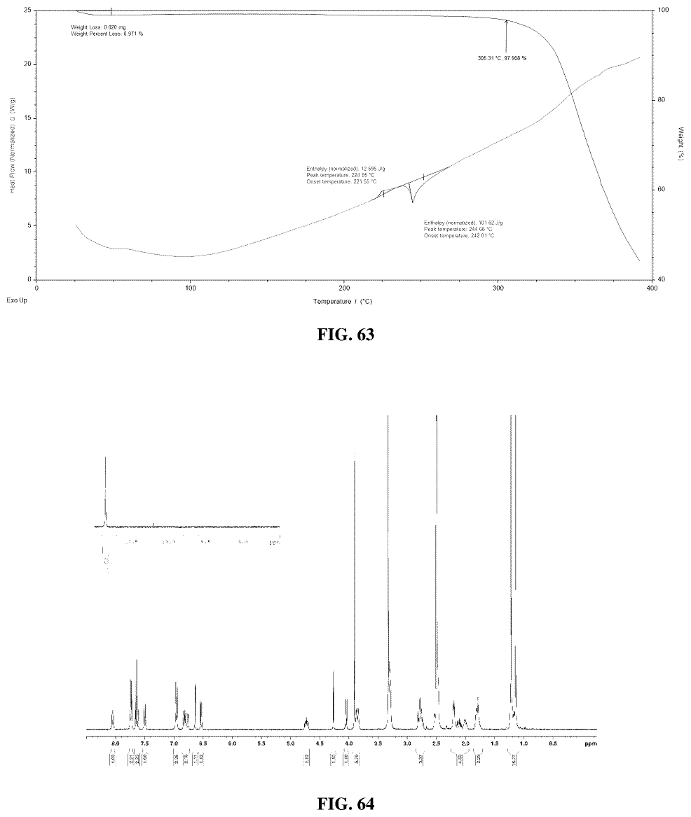

In some embodiments, the present disclosure provides solid forms of Compound A, e.g., crystalline forms of Compound A free base Pattern 2. In some embodiments, the Compound A free base Pattern 2 XRPD profile is substantially similar to that shown in FIG. 87. In some embodiments, the Compound A free base Pattern 2 FT-IR spectrum is substantially similar to that shown in FIG. 42. In some embodiments, the Compound A free base Pattern 2 ¹H NMR spectrum is substantially similar to that shown in FIG. 64. In some embodiments, the Compound A free base Pattern 2 TGA profile is substantially similar to that shown in FIG. 41 or FIG. 63. In some embodiments, the Compound A free base Pattern 2 DSC profile is substantially similar to that shown in FIG. 41 or FIG. 63.

In some embodiments, the solid form of Compound A free base Pattern 2 is crystalline Compound A free base Pattern 2 characterized by two or three XRPD signals selected from the group consisting of 14.4 °2θ, 19.1 °2θ, and 15.8 °2θ (±0.2 °2θ; ±0.1 °2θ; or ±0.0 °2θ; Cu Kα1 radiation). In some embodiments, the solid form of Compound A free base Pattern 2 is crystalline Compound A free base Pattern 2 characterized by XRPD signals at 14.4 °2θ, 19.1 °2θ, and 15.8 °2θ (±0.2 °2θ; ±0.1 °2θ; or ±0.0 °2θ; Cu Kα1 radiation).

In some embodiments, the solid form of Compound A free base Pattern 2 is crystalline Compound A free base Pattern 2 characterized by two or more, or three or more XRPD signals selected from the group consisting of 14.4 °2θ, 19.1 °2θ, 15.8 °2θ, 16.2 °2θ, and 23.5 °2θ (±0.2 °2θ; ±0.1 °2θ; or ±0.0 °2θ; Cu Kα1 radiation). In some embodiments, the solid form of Compound A free base Pattern 2 is crystalline Compound A free base Pattern 2 characterized by XRPD signals at 14.4 °2θ, 19.1 °2θ, 15.8 °2θ, 16.2 °2θ, and 23.5 °2θ (±0.2 °2θ; ±0.1 °2θ; or ±0.0 °2θ; Cu Kα1 radiation).

In some embodiments, the solid form of Compound A free base Pattern 2 is crystalline Compound A free base Pattern 2 characterized by two or more, or three or more XRPD signals selected from the group consisting of 14.4 °2θ, 19.1 °2θ, 15.8 °2θ, 16.2 °2θ, 23.5 °2θ, 15.1 °2θ, and 20.0 °2θ (±0.2 °2θ; ±0.1 °2θ; or ±0.0 °2θ; Cu Kα1 radiation). In some embodiments, the solid form of Compound A free base Pattern 2 is crystalline Compound A free base Pattern 2 characterized by XRPD signals at 14.4 °2θ, 19.1 °2θ, 15.8 °2θ, 16.2 °2θ, 23.5 °2θ, 15.1 °2θ, and 20.0 °2θ (±0.2 °2θ; ±0.1 °2θ; or ±0.0 °2θ; Cu Kα1 radiation).

In some embodiments, the solid form of Compound A free base Pattern 2 is crystalline Compound A free base Pattern 2 characterized by two or more, or three or more XRPD signals selected from the group consisting of 14.4 °2θ, 19.1 °2θ, 15.8 °2θ, 16.2 °2θ, 23.5 °2θ, 15.1 °2θ, 20.0 °2θ, 18.9 °2θ, 14.1 °2θ, and 15.6 °2θ (±0.2 °2θ; ±0.1 °2θ; or ±0.0 °2θ; Cu Kα1 radiation). In some embodiments, the solid form of Compound A free base Pattern 2 is crystalline Compound A free base Pattern 2 characterized by XRPD signals at 14.4 °2θ, 19.1 °2θ, 15.8 °2θ, 16.2 °2θ, 23.5 °2θ, 15.1 °2θ, 20.0 °2θ, 18.9 °2θ, 14.1 °2θ, and 15.6 °2θ (±0.2 °2θ; ±0.1 °2θ; or ±0.0 °2θ; Cu Kα1 radiation).

In some embodiments, the crystalline Compound A free base Pattern 2 is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, or forty XRPD signals selected from those set forth in Table 2.

TABLE 2
XRPD peak table of Compound A free base Pattern 2

| | Pos. | d-spacing | Height | Rel. Int. |
|---|---|---|---|---|
| No. | [° 2θ] | [Å] | [cts] | [%] |
| 1 | 3.5938 | 24.58631 | 471.26 | 6.74 |
| 2 | 6.3918 | 13.82845 | 370.61 | 5.30 |
| 3 | 10.8342 | 8.16620 | 101.22 | 1.45 |
| 4 | 12.4874 | 7.08856 | 202.93 | 2.90 |
| 5 | 13.1596 | 6.72796 | 328.63 | 4.70 |
| 6 | 13.8368 | 6.40018 | 585.70 | 8.38 |
| 7 | 14.1142 | 6.27501 | 1463.28 | 20.93 |
| 8 | 14.3971 | 6.15234 | 6990.97 | 100 |
| 9 | 15.1415 | 5.85148 | 1990.42 | 28.47 |
| 10 | 15.5564 | 5.69637 | 1276.45 | 18.26 |
| 11 | 15.7884 | 5.61318 | 2452.85 | 35.09 |
| 12 | 16.1703 | 5.48145 | 2435.14 | 34.83 |
| 13 | 16.6720 | 5.31762 | 172.04 | 2.46 |
| 14 | 17.2093 | 5.15277 | 270.44 | 3.87 |
| 15 | 17.4536 | 5.08121 | 191.78 | 2.74 |
| 16 | 18.6940 | 4.74676 | 599.68 | 8.58 |
| 17 | 18.9051 | 4.69424 | 1571.65 | 22.48 |
| 18 | 19.1353 | 4.63828 | 2498.87 | 35.74 |
| 19 | 19.3359 | 4.59061 | 1067.30 | 15.27 |
| 20 | 19.9557 | 4.44941 | 1696.27 | 24.26 |
| 21 | 20.4790 | 4.33688 | 93.58 | 1.34 |
| 22 | 21.2716 | 4.17704 | 874.54 | 12.51 |
| 23 | 21.4850 | 4.13602 | 756.05 | 10.81 |
| 24 | 21.8580 | 4.06292 | 644.56 | 9.22 |
| 25 | 21.9276 | 4.05354 | 588.96 | 8.42 |
| 26 | 22.1715 | 4.00950 | 409.04 | 5.85 |
| 27 | 23.0424 | 3.85988 | 160.96 | 2.30 |
| 28 | 23.5394 | 3.77951 | 2160.71 | 30.91 |
| 29 | 24.0647 | 3.69817 | 353.88 | 5.06 |
| 30 | 24.3750 | 3.65180 | 637.30 | 9.12 |
| 31 | 25.2690 | 3.52459 | 606.07 | 8.67 |
| 32 | 26.1682 | 3.40549 | 127.50 | 1.82 |
| 33 | 26.5622 | 3.35586 | 238.17 | 3.41 |
| 34 | 27.8794 | 3.20023 | 550.50 | 7.87 |
| 35 | 28.4715 | 3.13501 | 139.12 | 1.99 |
| 36 | 29.0143 | 3.07758 | 457.90 | 6.55 |
| 37 | 29.8633 | 2.99200 | 192.73 | 2.76 |
| 38 | 30.3918 | 2.94116 | 127.53 | 1.82 |
| 39 | 31.4577 | 2.84389 | 222.04 | 3.18 |
| 40 | 32.4282 | 2.76096 | 63.32 | 0.91 |

### Compound A Free Base Pattern 3

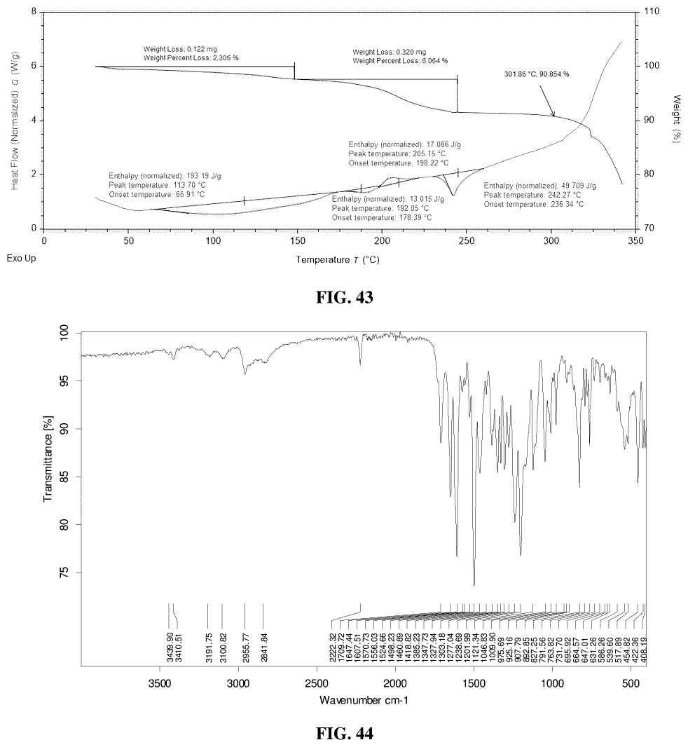

In some embodiments, the present disclosure provides solid forms of Compound A, e.g., crystalline forms of Compound A free base Pattern 3. In some embodiments, the Compound A free base Pattern 3 XRPD profile is substantially similar to that shown in FIG. 88. In some embodiments, the Compound A free base Pattern 3 FT-IR spectrum is substantially similar to that shown in FIG. 44. In some embodiments, the Compound A free base Pattern 3 TGA profile is substantially similar to that shown in FIG. 43. In some embodiments, the Compound A free base Pattern 3 DSC profile is substantially similar to that shown in FIG. 43.

In some embodiments, the solid form of Compound A free base Pattern 3 is crystalline Compound A free base Pattern 3 characterized by two or three XRPD signals selected from the group consisting of 20.6 °2θ, 16.1 °2θ, and 16.3 °2θ (±0.2 °2θ; ±0.1 °2θ; or ±0.0 °2θ; Cu Kα1 radiation). In some embodiments, the solid form of Compound A free base Pattern 3 is crystalline Compound A free base Pattern 3 characterized by XRPD signals at 20.6 °2θ, 16.1 °2θ, and 16.3 °2θ (±0.2 °2θ; ±0.1 °2θ; or ±0.0 °2θ; Cu Kα1 radiation).

In some embodiments, the solid form of Compound A free base Pattern 3 is crystalline Compound A free base Pattern 3 characterized by two or more, or three or more XRPD signals selected from the group consisting of 20.6 °2θ, 16.1 °2θ, 16.3 °2θ, 17.3 °2θ, and 16.8 °2θ (±0.2 °2θ; ±0.1 °2θ; or ±0.0 °2θ; Cu Kα1 radiation). In some embodiments, the solid form of Compound A free base Pattern 3 is crystalline Compound A free base Pattern 3 characterized by XRPD signals at 20.6 °2θ, 16.1 °2θ, 16.3 °2θ, 17.3 °2θ, and 16.8 °2θ (±0.2 °2θ; ±0.1 °2θ; or ±0.0 °2θ; Cu Kα1 radiation).

In some embodiments, the solid form of Compound A free base Pattern 3 is crystalline Compound A free base Pattern 3 characterized by two or more, or three or more XRPD signals selected from the group consisting of 20.6 °2θ, 16.1 °2θ, 16.3 °2θ, 17.3 °2θ, 16.8 °2θ, 18.1 °2θ, and 18.7 °2θ (±0.2 °2θ; ±0.1 °2θ; or ±0.0 °2θ; Cu Kα1 radiation). In some embodiments, the solid form of Compound A free base Pattern 3 is crystalline Compound A free base Pattern 3 characterized by XRPD signals at 20.6 °2θ, 16.1 °2θ, 16.3 °2θ, 17.3 °2θ, 16.8 °2θ, 18.1 °2θ, and 18.7 °2θ (±0.2 °2θ; ±0.1 °2θ; or ±0.0 °2θ; Cu Kα1 radiation).

In some embodiments, the solid form of Compound A free base Pattern 3 is crystalline Compound A free base Pattern 3 characterized by two or more, or three or more XRPD signals selected from the group consisting of 20.6 °2θ, 16.1 °2θ, 16.3 °2θ, 17.3 °2θ, 16.8 °2θ, 18.1 °2θ, 18.7 °2θ, 19.4 °2θ, 15.2 °2θ, and 22.1 °2θ (±0.2 °2θ; ±0.1 °2θ; or ±0.0 °2θ; Cu Kα1 radiation). In some embodiments, the solid form of Compound A free base Pattern 3 is crystalline Compound A free base Pattern 3 characterized by XRPD signals at 20.6 °2θ, 16.1 °2θ, 16.3 °2θ, 17.3 °2θ, 16.8 °2θ, 18.1 °2θ, 18.7 °2θ, 19.4 °2θ, 15.2 °2θ, and 22.1 °2θ (±0.2 °2θ; ±0.1 °2θ; or ±0.0 °2θ; Cu Kα1 radiation).

In some embodiments, the crystalline Compound A free base Pattern 3 is characterized by one, two, three, four, five, six, seven, eight, nine, ten, eleven, twelve, thirteen, fourteen, fifteen, sixteen, seventeen, eighteen, or nineteen XRPD signals selected from those set forth in Table 3.

TABLE 3
XRPD peak table of free base Pattern 3

| | Pos. | d-spacing | Height | Rel. Int. |
|---|---|---|---|---|
| No. | [° 2θ] | [Å] | [cts] | [%] |
| 1 | 3.1533 | 28.01991 | 199.81 | 22.26 |
| 2 | 5.1360 | 17.20662 | 133.94 | 14.92 |
| 3 | 6.4839 | 13.63215 | 54.37 | 6.06 |
| 4 | 7.2865 | 12.13234 | 44.57 | 4.97 |
| 5 | 10.9326 | 8.09293 | 142.82 | 15.91 |
| 6 | 12.3928 | 7.14247 | 93.30 | 10.39 |
| 7 | 15.1920 | 5.83216 | 398.19 | 44.36 |
| 8 | 16.0588 | 5.51926 | 883.64 | 98.44 |
| 9 | 16.3349 | 5.42659 | 773.87 | 86.21 |
| 10 | 16.7671 | 5.28766 | 592.30 | 65.98 |
| 11 | 17.3470 | 5.11218 | 695.50 | 77.48 |
| 12 | 18.0762 | 4.90757 | 585.33 | 65.21 |
| 13 | 18.7039 | 4.74428 | 585.40 | 65.21 |
| 14 | 19.3582 | 4.58537 | 475.42 | 52.96 |
| 15 | 20.5831 | 4.31517 | 897.66 | 100 |
| 16 | 22.0559 | 4.03025 | 361.08 | 40.22 |
| 17 | 24.0039 | 3.70741 | 212.30 | 23.65 |
| 18 | 25.3280 | 3.51651 | 225.68 | 25.14 |
| 19 | 27.4228 | 3.25246 | 95.55 | 10.64 |

### Compound A Free Base Pattern 4

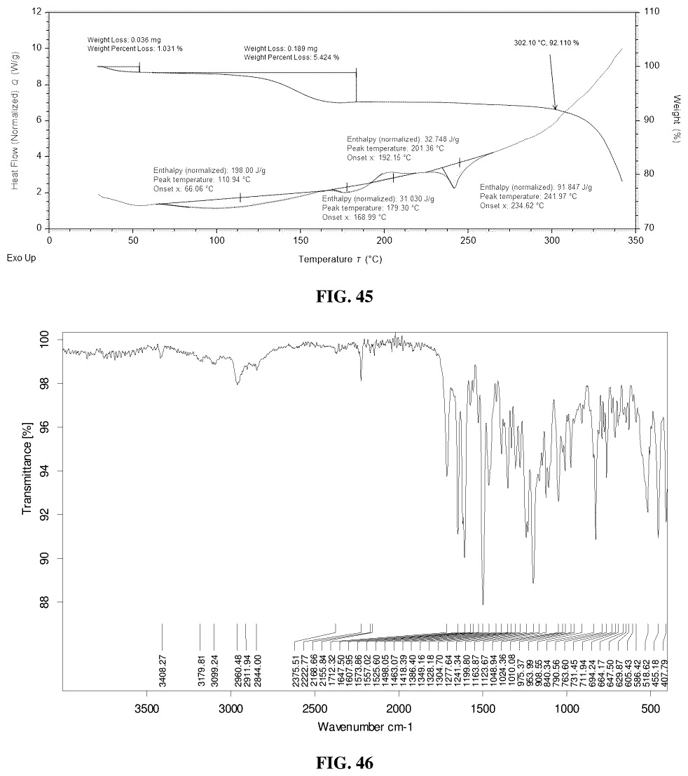

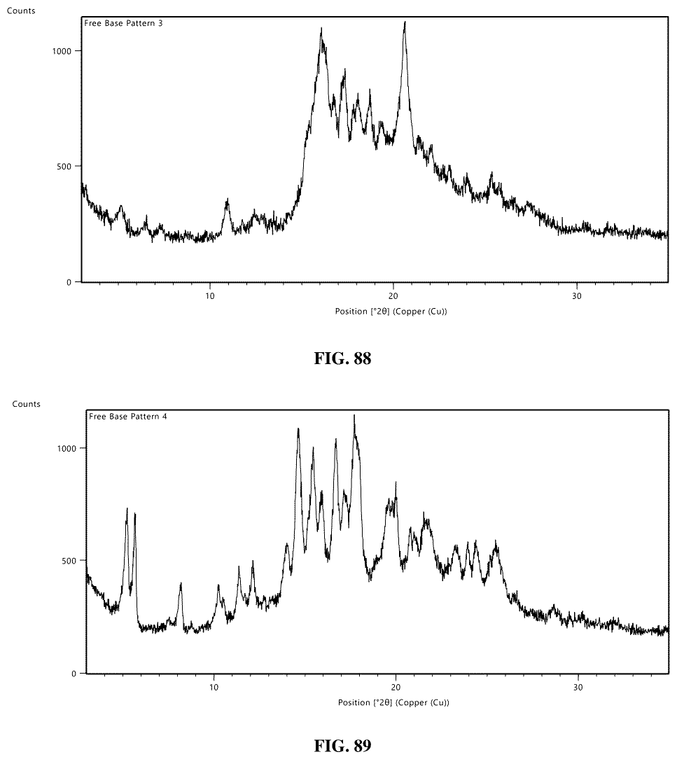

In some embodiments, the present disclosure provides solid forms of Compound A, e.g., crystalline forms of Compound A free base Pattern 4. In some embodiments, the Compound A free base Pattern 4 XRPD profile is substantially similar to that shown in FIG. 89. In some embodiments, the Compound A free base Pattern 4 FT-IR spectrum is substantially similar to that shown in FIG. 46. In some embodiments, the Compound A free base Pattern 4 TGA profile is substantially similar to that shown in FIG. 45. In some embodiments, the Compound A free base Pattern 4 DSC profile is substantially similar to that shown in FIG. 45.

In some embodiments, the solid form of Compound A free base Pattern 4 is crystalline Compound A free base Pattern 4 characterized by two or three XRPD signals selected from the group consisting of 14.6 °2θ, 17.7 °2θ, and 16.7 °2θ (±0.2 °2θ; ±0.1 °2θ; or ±0.0 °2θ; Cu Kα1 radiation). In some embodiments, the solid form of Compound A free base Pattern 4 is crystalline Compound A free base Pattern 4 characterized by XRPD signals at 14.6 °2θ, 17.7 °2θ, and 16.7 °2θ (±0.2 °2θ; ±0.1 °2θ; or ±0.0 °2θ; Cu Kα1 radiation).

In some embodiments, the solid form of Compound A free base Pattern 4 is crystalline Compound A free base Pattern 4 characterized by two or more, or three or more XRPD signals selected from the group consisting of 14.6 °2θ, 17.7 °2θ, 16.7 °2θ, 15.5 °2θ, and 18.0 °2θ (±0.2 °2θ; ±0.1 °2θ; or ±0.0 °2θ; Cu Kα1 radiation). In some embodiments, the solid form of Compound A free base Pattern 4 is crystalline Compound A free base Pattern 4 characterized by XRPD signals at 14.6 °2θ, 17.7 °2θ, 16.7 °2θ, 15.5 °2θ, and 18.0 °2θ (±0.2 °2θ; ±0.1 °2θ; or ±0.0 °2θ; Cu Kα1 radiation).

In some embodiments, the solid form of Compound A free base Pattern 4 is crystalline Compound A free base Pattern 4 characterized by two or more, or three or more XRPD signals selected from the group consisting of 14.6 °2θ, 17.7 °2θ, 16.7 °2θ, 15.5 °2θ, 18.0 °2θ, 17.2 °2θ, and 20.0 °2θ (±0.2 °2θ; ±0.1 °2θ; or ±0.0 °2θ; Cu Kα1 radiation). In some embodiments, the solid form of Compound A free base Pattern 4 is crystalline Compound A free base Pattern 4 characterized by XRPD signals at 14.6 °2θ, 17.7 °2θ, 16.7 °2θ, 15.5 °2θ, 18.0 °2θ, 17.2 °2θ, and 20.0 °2θ (±0.2 °2θ; ±0.1 °2θ; or ±0.0 °2θ; Cu Kα1 radiation).

In some embodiments, the solid form of Compound A free base Pattern 4 is crystalline Compound A free base Pattern 4 characterized by two or more, or three or more XRPD signals selected from the group consisting of 14.6 °2θ, 17.7 °2θ, 16.7 °2θ, 15.5 °2θ, 18.0 °2θ, 17.2 °2θ, 20.0 °2θ, 15.9 °2θ, 19.5 °2θ, and 5.2 °2θ (±0.2 °2θ; ±0.1 °2θ; or ±0.0 °2θ; Cu Kα1 radiation). In some embodiments, the solid form of Compound A free base Pattern 4 is crystalline Compound A free base Pattern 4 characterized by XRPD signals at 14.6 °2θ, 17.7 °2θ, 16.7 °2θ, 15.5 °2θ, 18.0 °2θ, 17.2 °2θ, 20.0 °2θ, 15.9 °2θ, 19.5 °2θ, and 5.2 °2θ (±0.2 °2θ; ±0.1 °2θ; or ±0.0 °2θ; Cu Kα1 radiation).

In some embodiments, the crystalline Compound A free base Pattern 4 is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four XRPD signals selected from those set forth in Table 4.

TABLE 4
XRPD peak table of free base Pattern 4

| | Pos. | d-spacing | Height | Rel. Int. |
|---|---|---|---|---|
| No. | [° 2θ] | [Å] | [cts] | [%] |
| 1 | 5.2452 | 16.84856 | 484.96 | 56.21 |
| 2 | 5.7086 | 15.48181 | 354.75 | 41.12 |
| 3 | 8.2080 | 10.77228 | 203.06 | 23.54 |
| 4 | 10.2494 | 8.63082 | 183.66 | 21.29 |
| 5 | 11.3960 | 7.76488 | 245.49 | 28.45 |
| 6 | 12.1283 | 7.29762 | 290.20 | 33.64 |
| 7 | 14.0643 | 6.29716 | 346.67 | 40.18 |
| 8 | 14.6344 | 6.05310 | 862.77 | 100 |
| 9 | 15.4641 | 5.73013 | 777.06 | 90.07 |
| 10 | 15.9300 | 5.56358 | 553.15 | 64.11 |
| 11 | 16.6892 | 5.31219 | 801.05 | 92.85 |
| 12 | 17.1572 | 5.16831 | 569.68 | 66.03 |
| 13 | 17.6836 | 5.01563 | 845.37 | 97.98 |
| 14 | 18.0122 | 4.92488 | 701.45 | 81.30 |
| 15 | 19.5261 | 4.54631 | 493.83 | 57.24 |
| 16 | 19.9947 | 4.44082 | 556.81 | 64.54 |
| 17 | 20.7873 | 4.27325 | 386.39 | 44.78 |
| 18 | 21.6418 | 4.10642 | 412.68 | 47.83 |
| 19 | 23.2647 | 3.82350 | 285.92 | 33.14 |
| 20 | 23.9294 | 3.71878 | 292.03 | 33.85 |
| 21 | 24.3788 | 3.65124 | 304.27 | 35.27 |
| 22 | 25.5365 | 3.48828 | 261.36 | 30.29 |
| 23 | 28.6029 | 3.12091 | 44.50 | 5.16 |
| 24 | 30.0998 | 2.96903 | 19.47 | 2.26 |

### Compound A Free Base Pattern 5

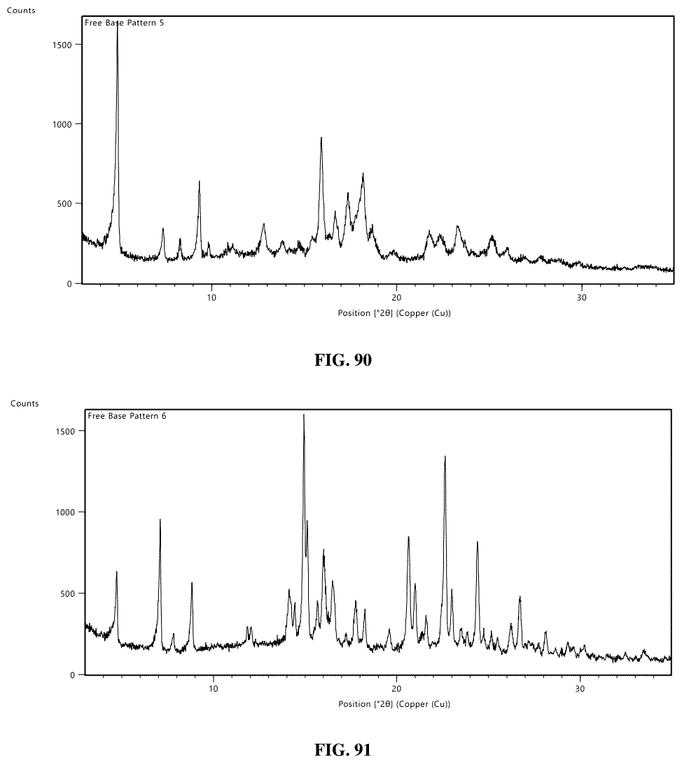

In some embodiments, the present disclosure provides solid forms of Compound A, e.g., crystalline forms of Compound A free base Pattern 5. In some embodiments, the Compound A free base Pattern 5 XRPD profile is substantially similar to that shown in FIG. 90.

In some embodiments, the solid form of Compound A free base Pattern 5 is crystalline Compound A free base Pattern 5 characterized by two or three XRPD signals selected from the group consisting of 14.9 °2θ, 22.6 °2θ, and 7.1 °2θ (±0.2 °2θ; ±0.1 °2θ; or ±0.0 °2θ; Cu Kα1 radiation). In some embodiments, the solid form of Compound A free base Pattern 5 is crystalline Compound A free base Pattern 5 characterized by XRPD signals at 14.9 °2θ, 22.6 °2θ, and 7.1 °2θ (±0.2 °2θ; ±0.1 °2θ; or ±0.0 °2θ; Cu Kα1 radiation).

In some embodiments, the solid form of Compound A free base Pattern 5 is crystalline Compound A free base Pattern 5 characterized by two or more, or three or more XRPD signals selected from the group consisting of 14.9 °2θ, 22.6 °2θ, 7.1 °2θ, 15.1 °2θ, and 20.6 °2θ (±0.2 °2θ; ±0.1 °2θ; or ±0.0 °2θ; Cu Kα1 radiation). In some embodiments, the solid form of Compound A free base Pattern 5 is crystalline Compound A free base Pattern 5 characterized by XRPD signals at 14.9 °2θ, 22.6 °2θ, 7.1 °2θ, 15.1 °2θ, and 20.6 °2θ (±0.2 °2θ; ±0.1 °2θ; or ±0.0 °2θ; Cu Kα1 radiation).

In some embodiments, the solid form of Compound A free base Pattern 5 is crystalline Compound A free base Pattern 5 characterized by two or more, or three or more XRPD signals selected from the group consisting of 14.9 °2θ, 22.6 °2θ, 7.1 °2θ, 15.1 °2θ, 20.6 °2θ, 24.4 °2θ, and 16.0 °2θ (±0.2 °2θ; ±0.1 °2θ; or ±0.0 °2θ; Cu Kα1 radiation). In some embodiments, the solid form of Compound A free base Pattern 5 is crystalline Compound A free base Pattern 5 characterized by XRPD signals at 14.9 °2θ, 22.6 °2θ, 7.1 °2θ, 15.1 °2θ, 20.6 °2θ, 24.4 °2θ, and 16.0 °2θ (±0.2 °2θ; ±0.1 °2θ; or ±0.0 °2θ; Cu Kα1 radiation).

In some embodiments, the solid form of Compound A free base Pattern 5 is crystalline Compound A free base Pattern 5 characterized by two or more, or three or more XRPD signals selected from the group consisting of 14.9 °2θ, 22.6 °2θ, 7.1 °2θ, 15.1 °2θ, 20.6 °2θ, 24.4 °2θ, 16.0 °2θ, 16.5 °2θ, 4.7 °2θ, and 21.0 °2θ (±0.2 °2θ; ±0.1 °2θ; or =0.0 °2θ; Cu Kα1 radiation). In some embodiments, the solid form of Compound A free base Pattern 5 is crystalline Compound A free base Pattern 5 characterized by XRPD signals at 14.9 °2θ, 22.6 °2θ, 7.1 °2θ, 15.1 °2θ, 20.6 °2θ, 24.4 °2θ, 16.0 °2θ, 16.5 °2θ, 4.7 °2θ, and 21.0 °2θ (±0.2 °2θ; ±0.1 °2θ; or ±0.0 °2θ; Cu Kα1 radiation).

In some embodiments, the crystalline Compound A free base Pattern 5 is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, or thirty-five XRPD signals 10 selected from those set forth in Table 5.

TABLE 5
XRPD peak table of free base Pattern 5

| | Pos. | d-spacing | Height | Rel. Int. |
|---|---|---|---|---|
| No. | [° 2θ] | [Å] | [cts] | [%] |
| 1 | 4.7342 | 18.66570 | 435.02 | 31.31 |
| 2 | 7.1042 | 12.44325 | 805.24 | 57.96 |
| 3 | 7.8131 | 11.31573 | 94.99 | 6.84 |
| 4 | 8.8298 | 10.01502 | 419.92 | 30.22 |
| 5 | 12.0610 | 7.33820 | 120.56 | 8.68 |
| 6 | 14.1220 | 6.27157 | 357.99 | 25.77 |
| 7 | 14.4339 | 6.13674 | 272.54 | 19.62 |
| 8 | 14.9492 | 5.92635 | 1389.39 | 100.00 |
| 9 | 15.1267 | 5.85718 | 766.21 | 55.15 |
| 10 | 15.6613 | 5.65842 | 298.52 | 21.49 |
| 11 | 15.9933 | 5.54173 | 592.82 | 42.67 |
| 12 | 16.4914 | 5.37543 | 439.69 | 31.65 |
| 13 | 17.2091 | 5.15284 | 88.73 | 6.39 |
| 14 | 17.7696 | 4.99155 | 293.33 | 21.11 |
| 15 | 18.2593 | 4.85878 | 234.14 | 16.85 |
| 16 | 19.5985 | 4.52969 | 135.75 | 9.77 |
| 17 | 20.6355 | 4.30433 | 720.53 | 51.86 |
| 18 | 20.9882 | 4.23279 | 427.27 | 30.75 |
| 19 | 21.6022 | 4.11385 | 216.50 | 15.58 |
| 20 | 22.6261 | 3.92996 | 1216.47 | 87.55 |
| 21 | 22.9965 | 3.86748 | 402.94 | 29.00 |
| 22 | 23.4912 | 3.78715 | 151.02 | 10.87 |
| 23 | 23.8393 | 3.73263 | 132.46 | 9.53 |
| 24 | 24.4027 | 3.64772 | 710.05 | 51.11 |
| 25 | 24.7362 | 3.59929 | 140.17 | 10.09 |
| 26 | 25.1602 | 3.53959 | 129.14 | 9.29 |
| 27 | 25.4791 | 3.49600 | 102.40 | 7.37 |
| 28 | 26.2265 | 3.39805 | 188.29 | 13.55 |
| 29 | 26.7367 | 3.33436 | 346.69 | 24.95 |
| 30 | 27.7251 | 3.21769 | 68.09 | 4.90 |
| 31 | 28.1086 | 3.17466 | 153.39 | 11.04 |
| 32 | 29.3312 | 3.04505 | 87.26 | 6.28 |
| 33 | 30.2210 | 2.95739 | 67.79 | 4.88 |
| 34 | 32.4755 | 2.75705 | 34.32 | 2.47 |
| 35 | 33.4594 | 2.67820 | 50.98 | 3.67 |

### Compound A Free Base Pattern 6

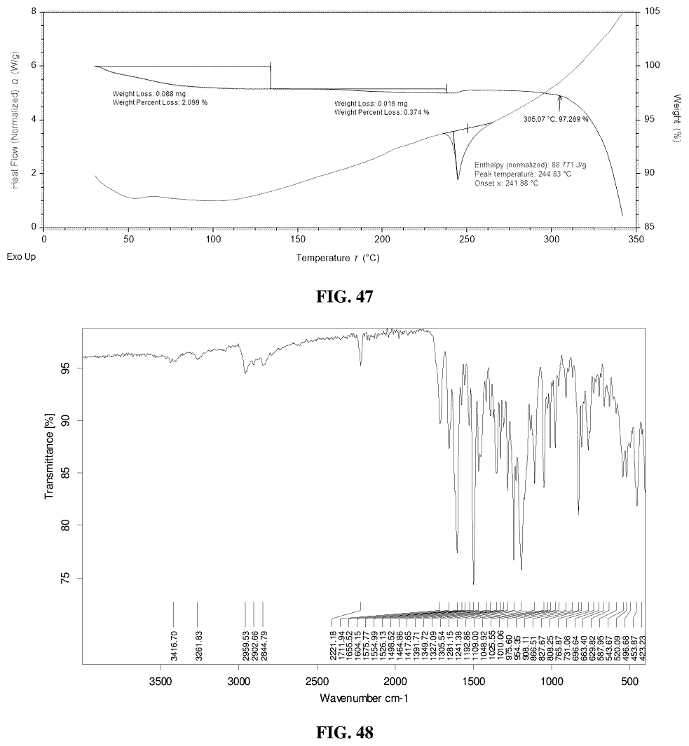

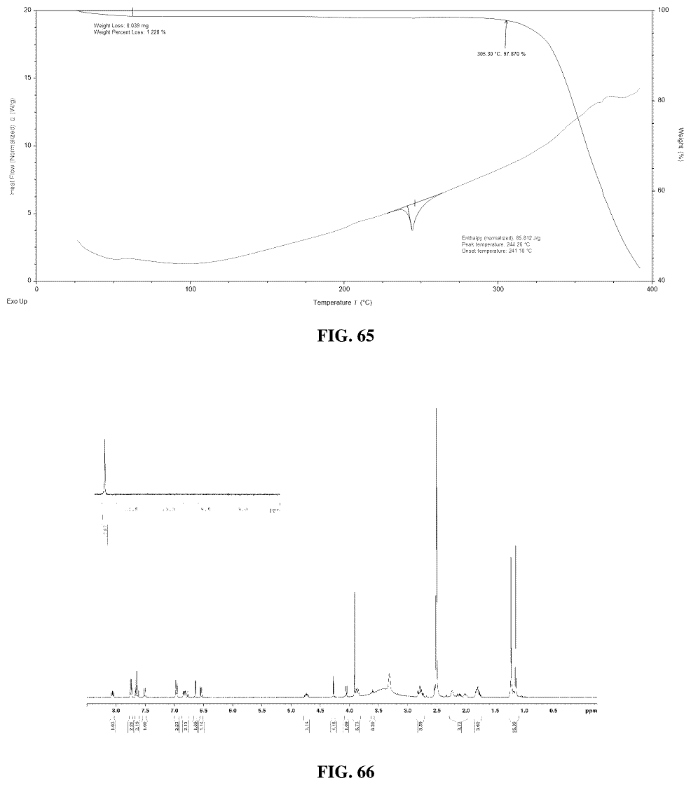

In some embodiments, the present disclosure provides solid forms of Compound A, e.g., crystalline forms of Compound A free base Pattern 6. In some embodiments, the Compound A free base Pattern 6 XRPD profile is substantially similar to that shown in FIG. 91. In some embodiments, the Compound A free base Pattern 6 FT-IR spectrum is substantially similar to that shown in FIG. 48. In some embodiments, the Compound A free base Pattern 6 ¹H NMR spectrum is substantially similar to that shown in FIG. 66. In some embodiments, the Compound A free base Pattern 6 TGA profile is substantially similar to that shown in FIG. 47 or FIG. 65. In some embodiments, the Compound A free base Pattern 6 DSC profile is substantially similar to that shown in FIG. 47 or FIG. 65.

In some embodiments, the solid form of Compound A free base Pattern 6 is crystalline Compound A free base Pattern 6 characterized by two or three XRPD signals selected from the group consisting of 3.5 °2θ, 3.6 °2θ, and 15.7 °2θ (±0.2 °2θ; ±0.1 °2θ; or ±0.0 °2θ; Cu Kα1 radiation). In some embodiments, the solid form of Compound A free base Pattern 6 is crystalline Compound A free base Pattern 6 characterized by XRPD signals at 3.5 °2θ, 3.6 °2θ, and 15.7 °2θ (±0.2 °2θ; ±0.1 °2θ; or ±0.0 °2θ; Cu Kα1 radiation).

In some embodiments, the solid form of Compound A free base Pattern 6 is crystalline Compound A free base Pattern 6 characterized by two or more, or three or more XRPD signals selected from the group consisting of 3.5 °2θ, 3.6 °2θ, 15.7 °2θ, 18.9 °2θ, and 23.7 °2θ (±0.2 °2θ; ±0.1 °2θ; or ±0.0 °2θ; Cu Kα1 radiation). In some embodiments, the solid form of Compound A free base Pattern 6 is crystalline Compound A free base Pattern 6 characterized by XRPD signals at 3.5 °2θ, 3.6 °2θ, 15.7 °2θ, 18.9 °2θ, and 23.7 °2θ (±0.2 °2θ; ±0.1 °2θ; or ±0.0 °2θ; Cu Kα1 radiation).

In some embodiments, the solid form of Compound A free base Pattern 6 is crystalline Compound A free base Pattern 6 characterized by two or more, or three or more XRPD signals selected from the group consisting of 3.5 °2θ, 3.6 °2θ, 15.7 °2θ, 18.9 °2θ, 23.7 °2θ, 16.2 °2θ, and 19.7 °2θ (±0.2 °2θ; ±0.1 °2θ; or ±0.0 °2θ; Cu Kα1 radiation). In some embodiments, the solid form of Compound A free base Pattern 6 is crystalline Compound A free base Pattern 6 characterized by XRPD signals at 3.5 °2θ, 3.6 °2θ, 15.7 °2θ, 18.9 °2θ, 23.7 °2θ, 16.2 °2θ, and 19.7 °2θ (±0.2 °2θ; ±0.1 °2θ; or ±0.0 °2θ; Cu Kα1 radiation).

In some embodiments, the solid form of Compound A free base Pattern 6 is crystalline Compound A free base Pattern 6 characterized by two or more, or three or more XRPD signals selected from the group consisting of 3.5 °2θ, 3.6 °2θ, 15.7 °2θ, 18.9 °2θ, 23.7 °2θ, 16.2 °2θ, 19.7 °2θ, 14.6 °2θ, 18.6 °2θ, and 15.1 °2θ (±0.2 °2θ; ±0.1 °2θ; or ±0.0 °2θ; Cu Kα1 radiation). In some embodiments, the solid form of Compound A free base Pattern 6 is crystalline Compound A free base Pattern 6 characterized by XRPD signals at 3.5 °2θ, 3.6 °2θ, 15.7 °2θ, 18.9 °2θ, 23.7 °2θ, 16.2 °2θ, 19.7 °2θ, 14.6 °2θ, 18.6 °2θ, and 15.1 °2θ (±0.2 °2θ; ±0.1 °2θ; or ±0.0 °2θ; Cu Kα1 radiation).

In some embodiments, the crystalline Compound A free base Pattern 6 is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, or thirty-six XRPD signals selected from those set forth in Table 6.

TABLE 6
XRPD peak table of free base Pattern 6

| | | | | Rel. Int. |
|---|---|---|---|---|
| No. | Pos. [°2θ] | d-spacing [Å] | Height [cts] | [%] |
| 1 | 3.4705 | 25.45949 | 1679.19 | 100 |
| 2 | 3.5648 | 24.78610 | 1608.67 | 95.80 |
| 3 | 5.2888 | 16.70955 | 105.77 | 6.30 |
| 4 | 6.2078 | 14.23787 | 274.03 | 16.32 |
| 5 | 6.9912 | 12.64407 | 68.31 | 4.07 |
| 6 | 8.6470 | 10.22631 | 75.89 | 4.52 |
| 7 | 9.9575 | 8.88317 | 93.78 | 5.58 |
| 8 | 10.7047 | 8.26472 | 201.20 | 11.98 |
| 9 | 11.4467 | 7.73062 | 124.96 | 7.44 |
| 10 | 12.0019 | 7.37421 | 132.67 | 7.90 |
| 11 | 12.4318 | 7.12015 | 204.83 | 12.20 |
| 12 | 12.9909 | 6.81498 | 303.98 | 18.10 |
| 13 | 13.3689 | 6.62311 | 150.21 | 8.95 |
| 14 | 14.1081 | 6.27769 | 282.11 | 16.80 |
| 15 | 14.3720 | 6.16303 | 454.21 | 27.05 |
| 16 | 14.5990 | 6.06768 | 546.02 | 32.52 |
| 17 | 15.0957 | 5.86916 | 483.71 | 28.81 |
| 18 | 15.7250 | 5.63567 | 1415.14 | 84.27 |
| 19 | 16.1687 | 5.48200 | 772.23 | 45.99 |
| 20 | 16.8342 | 5.26674 | 216.30 | 12.88 |
| 21 | 18.6367 | 4.76123 | 530.46 | 31.59 |
| 22 | 18.9323 | 4.68756 | 1027.73 | 61.20 |
| 23 | 19.3543 | 4.58628 | 442.11 | 26.33 |
| 24 | 19.6714 | 4.51305 | 757.73 | 45.12 |
| 25 | 21.0474 | 4.22101 | 448.20 | 26.69 |
| 26 | 21.8493 | 4.06788 | 446.56 | 26.59 |
| 27 | 23.0355 | 3.86103 | 280.34 | 16.69 |
| 28 | 23.6761 | 3.75799 | 980.03 | 58.36 |
| 29 | 24.5613 | 3.62452 | 326.92 | 19.47 |
| 30 | 25.1952 | 3.53475 | 231.17 | 13.77 |
| 31 | 26.0205 | 3.42448 | 214.65 | 12.78 |
| 32 | 27.6762 | 3.22326 | 167.57 | 9.98 |
| 33 | 28.5253 | 3.12922 | 147.44 | 8.78 |
| 34 | 29.0560 | 3.07326 | 238.55 | 14.21 |
| 35 | 31.5100 | 2.83929 | 59.23 | 3.53 |
| 36 | 32.4043 | 2.76295 | 49.20 | 2.93 |

### Compound A Free Base Pattern 7

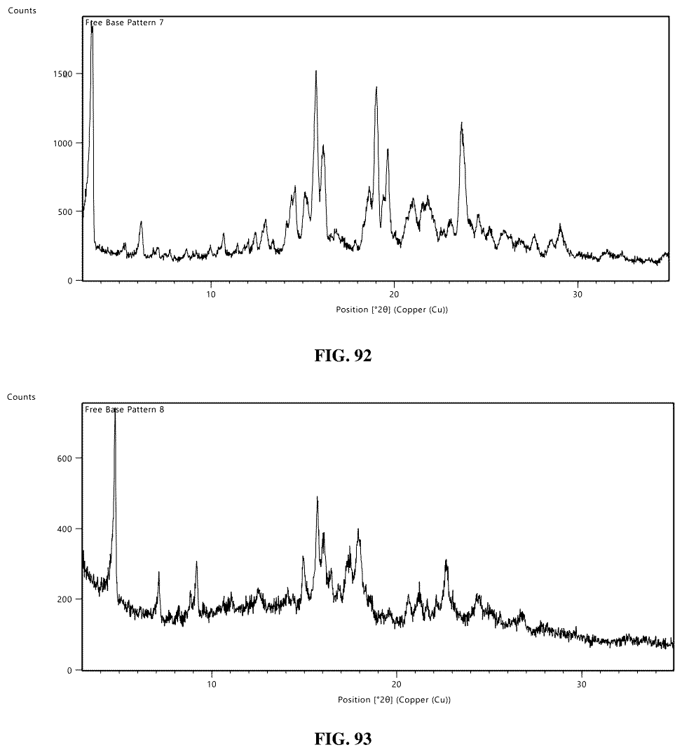

In some embodiments, the present disclosure provides solid forms of Compound A, e.g., crystalline forms of Compound A free base Pattern 7. In some embodiments, the Compound A free base Pattern 7 XRPD profile is substantially similar to that shown in FIG. 92.

In some embodiments, the solid form of Compound A free base Pattern 7 is crystalline Compound A free base Pattern 7 characterized by two or three XRPD signals selected from the group consisting of 4.8 °2θ, 15.7 °2θ, and 17.9 °2θ (±0.2 °2θ; ±0.1 °2θ; or ±0.0 °2θ; Cu Kα1 radiation). In some embodiments, the solid form of Compound A free base Pattern 7 is crystalline Compound A free base Pattern 7 characterized by XRPD signals at 4.8 °2θ, 15.7 °2θ, and 17.9 °2θ (±0.2 °2θ; ±0.1 °2θ; or ±0.0 °2θ; Cu Kα1 radiation).

In some embodiments, the solid form of Compound A free base Pattern 7 is crystalline Compound A free base Pattern 7 characterized by two or more, or three or more XRPD signals selected from the group consisting of 4.8 °2θ, 15.7 °2θ, 17.9 °2θ, 16.0 °2θ, and 17.4 °2θ (±0.2 °2θ; ±0.1 °2θ; or ±0.0 °2θ; Cu Kα1 radiation). In some embodiments, the solid form of Compound A free base Pattern 7 is crystalline Compound A free base Pattern 7 characterized by XRPD signals at 4.8 °2θ, 15.7 °2θ, 17.9 °2θ, 16.0 °2θ, and 17.4 °2θ (±0.2 °2θ; ±0.1 °2θ; or ±0.0 °2θ; Cu Kα1 radiation).

In some embodiments, the solid form of Compound A free base Pattern 7 is crystalline Compound A free base Pattern 7 characterized by two or more, or three or more XRPD signals selected from the group consisting of 4.8 °2θ, 15.7 °2θ, 17.9 °2θ, 16.0 °2θ, 17.4 °2θ, 15.0 °2θ, and 22.7 °2θ (±0.2 °2θ; ±0.1 °2θ; or ±0.0 °2θ; Cu Kα1 radiation). In some embodiments, the solid form of Compound A free base Pattern 7 is crystalline Compound A free base Pattern 7 characterized by XRPD signals at 4.8 °2θ, 15.7 °2θ, 17.9 °2θ, 16.0 °2θ, 17.4 °2θ, 15.0 °2θ, and 22.7 °2θ (±0.2 °2θ; ±0.1 °2θ; or ±0.0 °2θ; Cu Kα1 radiation).

In some embodiments, the solid form of Compound A free base Pattern 7 is crystalline Compound A free base Pattern 7 characterized by two or more, or three or more XRPD signals selected from the group consisting of 4.8 °2θ, 15.7 °2θ, 17.9 °2θ, 16.0 °2θ, 17.4 °2θ, 15.0 °2θ, 22.7 °2θ, 9.2 °2θ, 16.4 °2θ, and 7.1 °2θ (±0.2 °2θ; ±0.1 °2θ; or ±0.0 °2θ; Cu Kα1 radiation). In some embodiments, the solid form of Compound A free base Pattern 7 is crystalline Compound A free base Pattern 7 characterized by XRPD signals at 4.8 °2θ, 15.7 °2θ, 17.9 °2θ, 16.0 °2θ, 17.4 °2θ, 15.0 °2θ, 22.7 °2θ, 9.2 °2θ, 16.4 °2θ, and 7.1 °2θ (±0.2 °2θ; ±0.1 °2θ; or ±0.0 °2θ; Cu Kα1 radiation).

In some embodiments, the crystalline Compound A free base Pattern 7 is characterized by one, two, three, four, five, six, seven, eight, nine, ten, eleven, twelve, thirteen, fourteen, fifteen, or sixteen XRPD signals selected from those set forth in Table 7.

TABLE 7
XRPD peak table of free base Pattern 7

| | | | | Rel. Int. |
|---|---|---|---|---|
| No. | Pos. [°2θ] | d-spacing [Å] | Height [cts] | [%] |
| 1 | 4.7846 | 18.46937 | 555.74 | 100 |
| 2 | 7.1460 | 12.37051 | 116.68 | 21.00 |
| 3 | 9.1907 | 9.62255 | 166.56 | 29.97 |
| 4 | 10.8425 | 8.15997 | 47.31 | 8.51 |
| 5 | 12.5754 | 7.03915 | 84.85 | 15.27 |
| 6 | 14.9522 | 5.92513 | 187.84 | 33.80 |
| 7 | 15.7216 | 5.63688 | 337.17 | 60.67 |
| 8 | 16.0360 | 5.52707 | 232.80 | 41.89 |
| 9 | 16.4450 | 5.39049 | 147.58 | 26.56 |
| 10 | 17.4437 | 5.08406 | 190.93 | 34.36 |
| 11 | 17.9433 | 4.94364 | 259.48 | 46.69 |
| 12 | 20.6178 | 4.30800 | 90.81 | 16.34 |
| 13 | 21.2453 | 4.18214 | 95.70 | 17.22 |
| 14 | 22.6860 | 3.91971 | 183.18 | 32.96 |
| 15 | 24.3570 | 3.65445 | 85.43 | 15.37 |
| 16 | 26.8207 | 3.32410 | 43.24 | 7.78 |

### Compound A Free Base Pattern 8

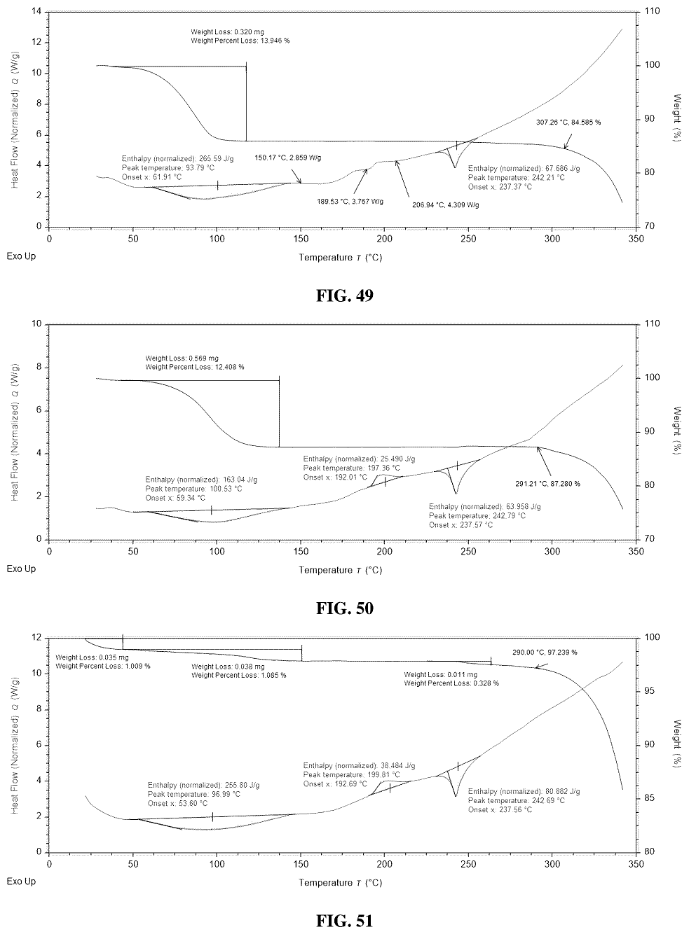

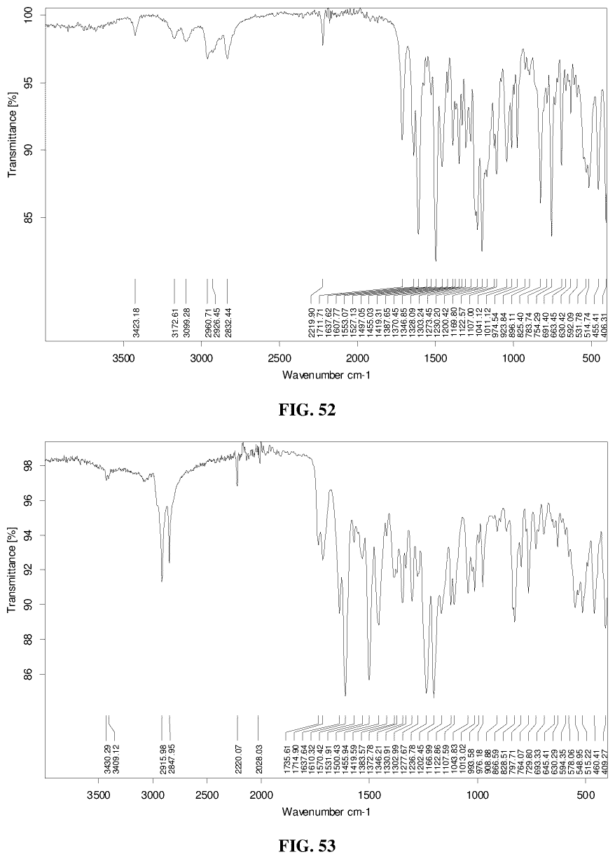

In some embodiments, the present disclosure provides solid forms of Compound A, e.g., crystalline forms of Compound A free base Pattern 8. In some embodiments, the Compound A free base Pattern 8 XRPD profile is substantially similar to that shown in FIG. 93. In some embodiments, the Compound A free base Pattern 8 FT-IR spectrum is substantially similar to that shown in FIG. 52 or FIG. 53. In some embodiments, the Compound A free base Pattern 8 TGA profile is substantially similar to that shown in FIG. 49, FIG. 50, or FIG. 51. In some embodiments, the Compound A free base Pattern 8 DSC profile is substantially similar to that shown in FIG. 49, FIG. 50, or FIG. 51.

In some embodiments, the solid form of Compound A free base Pattern 8 is crystalline Compound A free base Pattern 8 characterized by two or three XRPD signals selected from the group consisting of 4.9 °2θ, 15.9 °2θ, and 18.2 °2θ (±0.2 °2θ; ±0.1 °2θ; or ±0.0 °2θ; Cu Kα1 radiation). In some embodiments, the solid form of Compound A free base Pattern 8 is crystalline Compound A free base Pattern 8 characterized by XRPD signals at 4.9 °2θ, 15.9 °2θ, and 18.2 °2θ (±0.2 °2θ; ±0.1 °2θ; or ±0.0 °2θ; Cu Kα1 radiation).

In some embodiments, the solid form of Compound A free base Pattern 8 is crystalline Compound A free base Pattern 8 characterized by two or more, or three or more XRPD signals selected from the group consisting of 4.9 °2θ, 15.9 °2θ, 18.2 °2θ, 9.3 °2θ, and 17.4 °2θ (±0.2 °2θ; ±0.1 °2θ; or ±0.0 °2θ; Cu Kα1 radiation). In some embodiments, the solid form of Compound A free base Pattern 8 is crystalline Compound A free base Pattern 8 characterized by XRPD signals at 4.9 °2θ, 15.9 °2θ, 18.2 °2θ, 9.3 °2θ, and 17.4 °2θ (±0.2 °2θ; ±0.1 °2θ; or ±0.0 °2θ; Cu Kα1 radiation).

In some embodiments, the solid form of Compound A free base Pattern 8 is crystalline Compound A free base Pattern 8 characterized by two or more, or three or more XRPD signals selected from the group consisting of 4.9 °2θ, 15.9 °2θ, 18.2 °2θ, 9.3 °2θ, 17.4 °2θ, 16.7 °2θ, and 23.3 °2θ (±0.2 °2θ; ±0.1 °2θ; or ±0.0 °2θ; Cu Kα1 radiation). In some embodiments, the solid form of Compound A free base Pattern 8 is crystalline Compound A free base Pattern 8 characterized by XRPD signals at 4.9 °2θ, 15.9 °2θ, 18.2 °2θ, 9.3 °2θ, 17.4 °2θ, 16.7 °2θ, and 23.3 °2θ (±0.2 °2θ; ±0.1 °2θ; or ±0.0 °2θ; Cu Kα1 radiation).

In some embodiments, the solid form of Compound A free base Pattern 8 is crystalline Compound A free base Pattern 8 characterized by two or more, or three or more XRPD signals selected from the group consisting of 4.9 °2θ, 15.9 °2θ, 18.2 °2θ, 9.3 °2θ, 17.4 °2θ, 16.7 °2θ, 23.3 °2θ, 12.8 °2θ, 18.7 °2θ, and 21.8 °2θ (±0.2 °2θ; ±0.1 °2θ; or ±0.0 °2θ; Cu Kα1 radiation). In some embodiments, the solid form of Compound A free base Pattern 8 is crystalline Compound A free base Pattern 8 characterized by XRPD signals at 4.9 °2θ, 15.9 °2θ, 18.2 °2θ, 9.3 °2θ, 17.4 °2θ, 16.7 °2θ, 23.3 °2θ, 12.8 °2θ, 18.7 °2θ, and 21.8 °2θ (±0.2 °2θ; ±0.1 °2θ; or ±0.0 °2θ; Cu Kα1 radiation).

In some embodiments, the crystalline Compound A free base Pattern 8 is characterized by one, two, three, four, five, six, seven, eight, nine, ten, eleven, twelve, thirteen, fourteen, fifteen, sixteen, seventeen, eighteen, nineteen, twenty, twenty-one, or twenty-two XRPD signals selected from those set forth in Table 8.

TABLE 8
XRPD peak table of free base Pattern 8

| | | | | Rel. Int. |
|---|---|---|---|---|
| No. | Pos. [°2θ] | d-spacing [Å] | Height [cts] | [%] |
| 1 | 4.9271 | 17.93558 | 1408.55 | 100 |
| 2 | 7.3721 | 11.99163 | 201.90 | 14.33 |
| 3 | 8.2925 | 10.66268 | 123.26 | 8.75 |
| 4 | 9.3164 | 9.49295 | 452.53 | 32.13 |
| 5 | 9.8468 | 8.98276 | 101.69 | 7.22 |
| 6 | 11.0294 | 8.02213 | 75.81 | 5.38 |
| 7 | 12.8200 | 6.90543 | 243.26 | 17.27 |
| 8 | 13.8335 | 6.40170 | 127.75 | 9.07 |
| 9 | 15.4236 | 5.74510 | 158.30 | 11.24 |
| 10 | 15.9237 | 5.56579 | 755.83 | 53.66 |
| 11 | 16.6601 | 5.32140 | 319.47 | 22.68 |
| 12 | 17.3530 | 5.11043 | 433.99 | 30.81 |
| 13 | 18.2047 | 4.87323 | 536.78 | 38.11 |
| 14 | 18.6651 | 4.75406 | 221.38 | 15.72 |
| 15 | 19.7961 | 4.48491 | 76.21 | 5.41 |
| 16 | 21.7500 | 4.08623 | 211.02 | 14.98 |
| 17 | 22.3220 | 3.98281 | 180.80 | 12.84 |
| 18 | 23.2977 | 3.81816 | 252.51 | 17.93 |
| 19 | 25.1152 | 3.54582 | 180.34 | 12.80 |
| 20 | 25.9684 | 3.43123 | 119.03 | 8.45 |
| 21 | 26.8884 | 3.31588 | 59.74 | 4.24 |
| 22 | 29.7854 | 2.99964 | 31.67 | 2.25 |

### Compound A Free Base Pattern 9

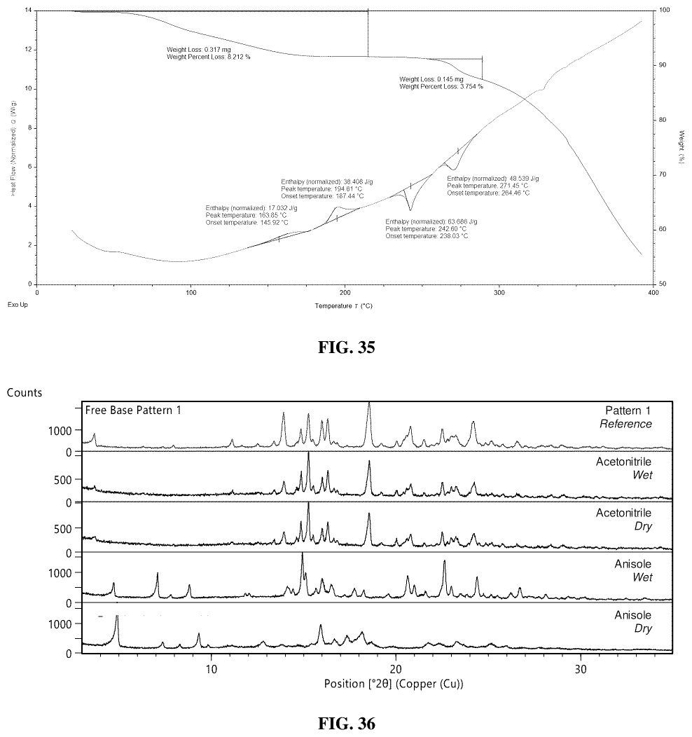

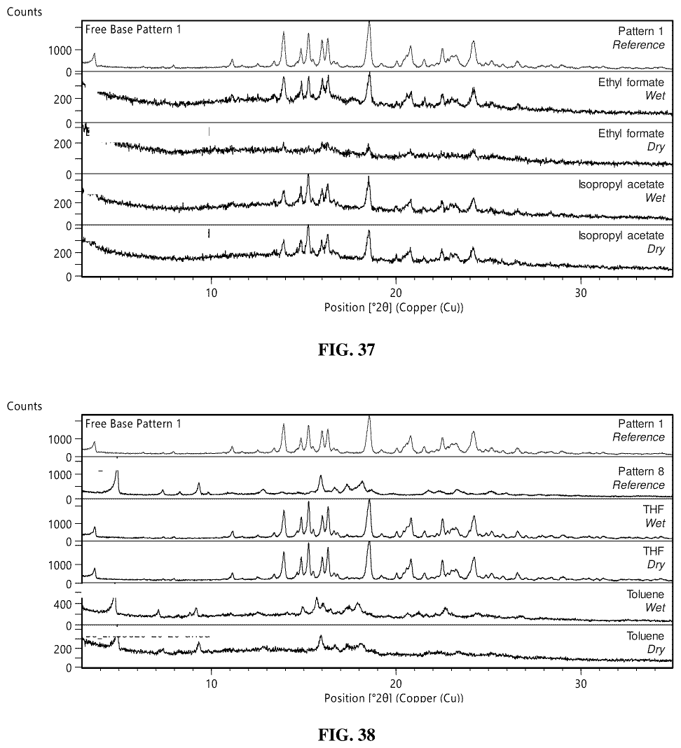

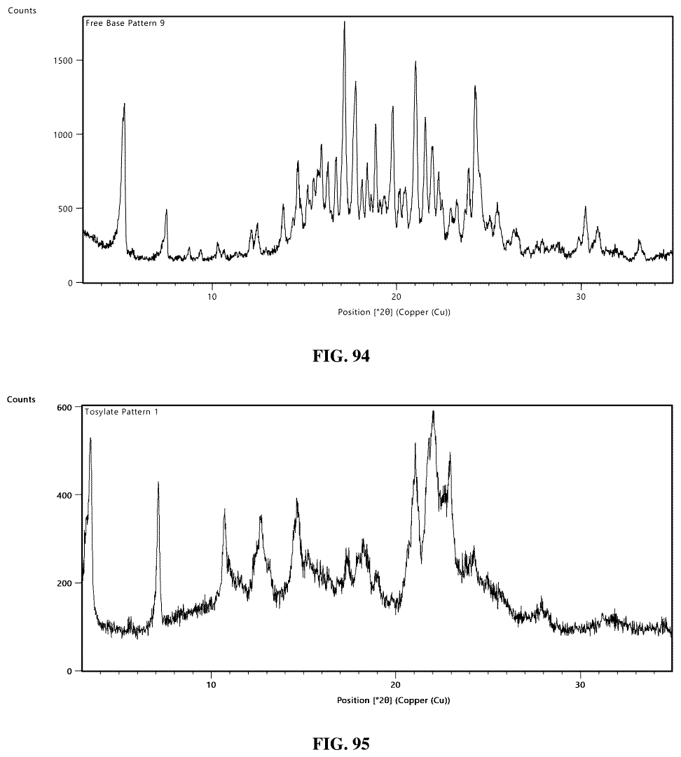

In some embodiments, the present disclosure provides solid forms of Compound A, e.g., crystalline forms of Compound A free base Pattern 9. In some embodiments, the Compound A free base Pattern 9 XRPD profile is substantially similar to that shown in FIG. 94. In some embodiments, the Compound A free base Pattern 9 TGA profile is substantially similar to that shown in FIG. 35. In some embodiments, the Compound A free base Pattern 9 DSC profile is substantially similar to that shown in FIG. 35.

In some embodiments, the solid form of Compound A free base Pattern 9 is crystalline Compound A free base Pattern 9 characterized by two or three XRPD signals selected from the group consisting of 17.2 °2θ, 21.0 °2θ, and 24.2 °2θ (±0.2 °2θ; ±0.1 °2θ; or ±0.0 °2θ; Cu Kα1 radiation). In some embodiments, the solid form of Compound A free base Pattern 9 is crystalline Compound A free base Pattern 9 characterized by XRPD signals at 17.2 °2θ, 21.0 °2θ, and 24.2 °2θ (±0.2 °2θ; ±0.1 °2θ; or ±0.0 °2θ; Cu Kα1 radiation).

In some embodiments, the solid form of Compound A free base Pattern 9 is crystalline Compound A free base Pattern 9 characterized by two or more, or three or more XRPD signals selected from the group consisting of 17.2 °2θ, 21.0 °2θ, 24.2 °2θ, 17.8 °2θ, and 19.8 °2θ (±0.2 °2θ; ±0.1 °2θ; or ±0.0 °2θ; Cu Kα1 radiation). In some embodiments, the solid form of Compound A free base Pattern 9 is crystalline Compound A free base Pattern 9 characterized by XRPD signals at 17.2 °2θ, 21.0 °2θ, 24.2 °2θ, 17.8 °2θ, and 19.8 °2θ (±0.2 °2θ; ±0.1 °2θ; or ±0.0 °2θ; Cu Kα1 radiation).

In some embodiments, the solid form of Compound A free base Pattern 9 is crystalline Compound A free base Pattern 9 characterized by two or more, or three or more XRPD signals selected from the group consisting of 17.2 °2θ, 21.0 °2θ, 24.2 °2θ, 17.8 °2θ, 19.8 °2θ, 5.3 °2θ, and 21.5 °2θ (±0.2 °2θ; ±0.1 °2θ; or ±0.0 °2θ; Cu Kα1 radiation). In some embodiments, the solid form of Compound A free base Pattern 9 is crystalline Compound A free base Pattern 9 characterized by XRPD signals at 17.2 °2θ, 21.0 °2θ, 24.2 °2θ, 17.8 °2θ, 19.8 °2θ, 5.3 °2θ, and 21.5 °2θ (±0.2 °2θ; ±0.1 °2θ; or ±0.0 °2θ; Cu Kα1 radiation).

In some embodiments, the solid form of Compound A free base Pattern 9 is crystalline Compound A free base Pattern 9 characterized by two or more, or three or more XRPD signals selected from the group consisting of 17.2 °2θ, 21.0 °2θ, 24.2 °2θ, 17.8 °2θ, 19.8 °2θ, 5.3 °2θ, 21.5 °2θ, 18.9 °2θ, 15.9 °2θ, and 21.9 °2θ (±0.2 °2θ; ±0.1 °2θ; or ±0.0 °2θ; Cu Kα1 radiation). In some embodiments, the solid form of Compound A free base Pattern 9 is crystalline Compound A free base Pattern 9 characterized by XRPD signals at 17.2 °2θ, 21.0 °2θ, 24.2 °2θ, 17.8 °2θ, 19.8 °2θ, 5.3 °2θ, 21.5 °2θ, 18.9 °2θ, 15.9 °2θ, and 21.9 °2θ (±0.2 °2θ; ±0.1 °2θ; or ±0.0 °2θ; Cu Kα1 radiation).

In some embodiments, the crystalline Compound A free base Pattern 9 is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, or forty-two XRPD signals selected from those set forth in Table 9.

TABLE 9
XRPD peak table of free base Pattern 9

| | | | | Rel. Int. |
|---|---|---|---|---|
| No. | Pos. [°2θ] | d-spacing [Å] | Height [cts] | [%] |
| 1 | 5.2587 | 16.80532 | 992.19 | 66.02 |
| 2 | 7.5444 | 11.71813 | 324.36 | 21.58 |
| 3 | 8.7729 | 10.07985 | 78.14 | 5.20 |
| 4 | 9.4016 | 9.40715 | 58.15 | 3.87 |
| 5 | 10.3019 | 8.58699 | 100.94 | 6.72 |
| 6 | 12.1271 | 7.29836 | 195.38 | 13.00 |
| 7 | 12.4455 | 7.11233 | 232.02 | 15.44 |
| 8 | 13.8319 | 6.40244 | 350.20 | 23.30 |
| 9 | 14.6420 | 6.04999 | 651.41 | 43.35 |
| 10 | 15.1567 | 5.84565 | 464.66 | 30.92 |
| 11 | 15.4935 | 5.71936 | 551.68 | 36.71 |
| 12 | 15.9189 | 5.56745 | 780.38 | 51.93 |
| 13 | 16.2899 | 5.44147 | 667.44 | 44.41 |
| 14 | 16.7350 | 5.29773 | 698.90 | 46.51 |
| 15 | 17.2001 | 5.15552 | 1502.83 | 100 |
| 16 | 17.7980 | 4.98364 | 1172.54 | 78.02 |
| 17 | 18.1364 | 4.89143 | 543.12 | 36.14 |
| 18 | 18.4086 | 4.81971 | 649.76 | 43.24 |
| 19 | 18.8565 | 4.70623 | 925.93 | 61.61 |
| 20 | 19.3271 | 4.59267 | 419.40 | 27.91 |
| 21 | 19.8046 | 4.48301 | 1046.15 | 69.61 |
| 22 | 20.1561 | 4.40561 | 455.07 | 30.28 |
| 23 | 20.5053 | 4.33137 | 448.81 | 29.86 |
| 24 | 21.0399 | 4.22251 | 1316.04 | 87.57 |
| 25 | 21.5464 | 4.12439 | 964.82 | 64.20 |
| 26 | 21.9024 | 4.05814 | 747.69 | 49.75 |
| 27 | 22.2780 | 3.99056 | 583.12 | 38.80 |
| 28 | 22.4942 | 3.95270 | 357.81 | 23.81 |
| 29 | 22.9214 | 3.87999 | 341.68 | 22.74 |
| 30 | 23.2816 | 3.82077 | 371.69 | 24.73 |
| 31 | 23.9028 | 3.72286 | 597.05 | 39.73 |
| 32 | 24.2477 | 3.67069 | 1187.96 | 79.05 |
| 33 | 25.0625 | 3.55316 | 279.90 | 18.62 |
| 34 | 25.4298 | 3.50267 | 344.69 | 22.94 |
| 35 | 26.3415 | 3.38347 | 190.53 | 12.68 |
| 36 | 26.5879 | 3.35267 | 147.05 | 9.78 |
| 37 | 27.1133 | 3.28889 | 68.60 | 4.56 |
| 38 | 27.8973 | 3.19821 | 115.46 | 7.68 |
| 39 | 28.6140 | 3.11972 | 72.51 | 4.82 |
| 40 | 30.2340 | 2.95615 | 343.46 | 22.85 |
| 41 | 30.8780 | 2.89595 | 204.79 | 13.63 |
| 42 | 33.1838 | 2.69980 | 111.90 | 7.45 |

### Compound A Tosylate Pattern 1

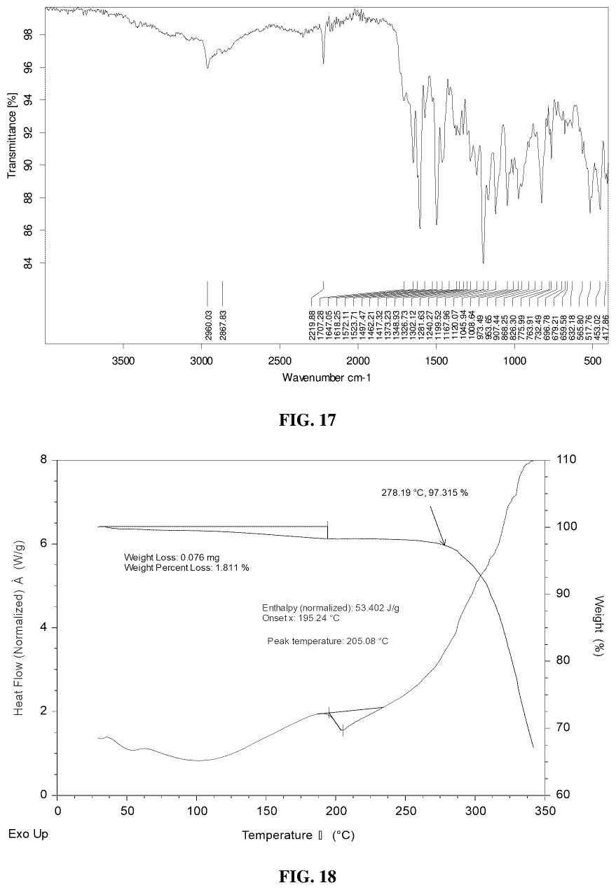

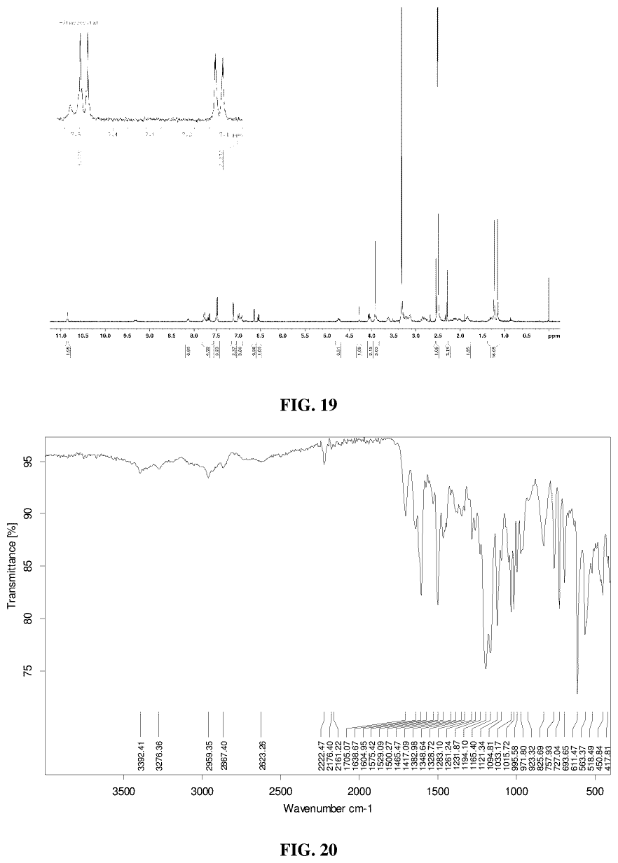

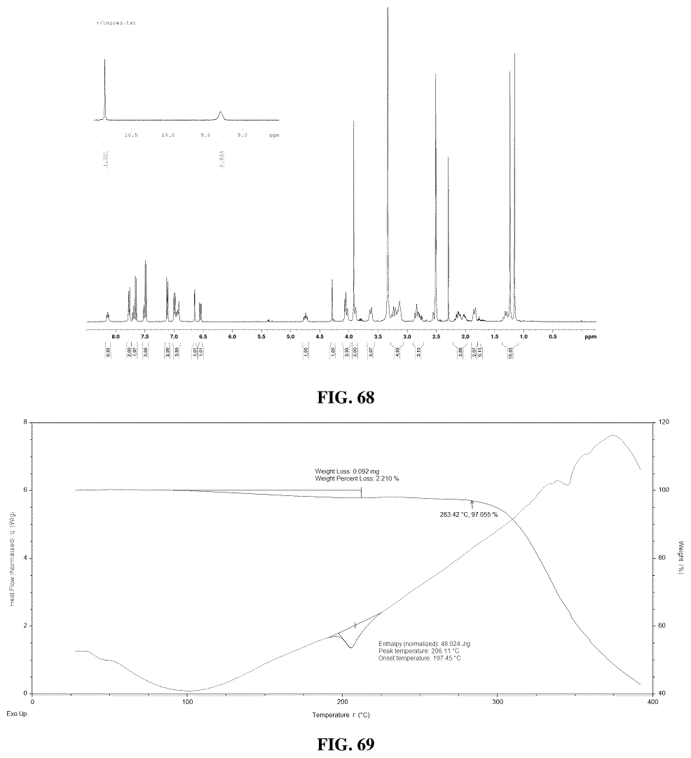

In some embodiments, the present disclosure provides solid forms of Compound A, e.g., crystalline forms of Compound A tosylate Pattern 1. In some embodiments, the Compound A tosylate Pattern 1 XRPD profile is substantially similar to that shown in FIG. 95. In some embodiments, the Compound A tosylate Pattern 1 FT-IR spectrum is substantially similar to that shown in FIG. 17. In some embodiments, the Compound A tosylate Pattern 1 ¹H NMR spectrum is substantially similar to that shown in FIG. 19 or FIG. 68. In some embodiments, the Compound A tosylate Pattern 1 TGA profile is substantially similar to that shown in FIG. 18 or FIG. 69. In some embodiments, the Compound A tosylate Pattern 1 DSC profile is substantially similar to that shown in FIG. 18 or FIG. 69.

In some embodiments, the solid form of Compound A tosylate Pattern 1 is crystalline Compound A tosylate Pattern 1 characterized by two or three XRPD signals selected from the group consisting of 3.5 °2θ, 22.0 °2θ, and 23.0 °2θ (±0.2 °2θ; ±0.1 °2θ; or ±0.0 °2θ; Cu Kα1 radiation). In some embodiments, the solid form of Compound A tosylate Pattern 1 is crystalline Compound A tosylate Pattern 1 characterized by XRPD signals at 3.5 °2θ, 22.0 °2θ, and 23.0 °2θ (±0.2 °2θ; ±0.1 °2θ; or ±0.0 °2θ; Cu Kα1 radiation).

In some embodiments, the solid form of Compound A tosylate Pattern 1 is crystalline Compound A tosylate Pattern 1 characterized by two or more, or three or more XRPD signals selected from the group consisting of 3.5 °2θ, 22.0 °2θ, 23.0 °2θ, 21.7 °2θ, and 7.2 °2θ (±0.2 °2θ; ±0.1 °2θ; or ±0.0 °2θ; Cu Kα1 radiation). In some embodiments, the solid form of Compound A tosylate Pattern 1 is crystalline Compound A tosylate Pattern 1 characterized by XRPD signals at 3.5 °2θ, 22.0 °2θ, 23.0 °2θ, 21.7 °2θ, and 7.2 °2θ (±0.2 °2θ; ±0.1 °2θ; or ±0.0 °2θ; Cu Kα1 radiation).

In some embodiments, the solid form of Compound A tosylate Pattern 1 is crystalline Compound A tosylate Pattern 1 characterized by two or more, or three or more XRPD signals selected from the group consisting of 3.5 °2θ, 22.0 °2θ, 23.0 °2θ, 21.7 °2θ, 7.2 °2θ, 21.0 °2θ, and 14.7 °2θ (±0.2 °2θ; ±0.1 °2θ; or ±0.0 °2θ; Cu Kα1 radiation). In some embodiments, the solid form of Compound A tosylate Pattern 1 is crystalline Compound A tosylate Pattern 1 characterized by XRPD signals at 3.5 °2θ, 22.0 °2θ, 23.0 °2θ, 21.7 °2θ, 7.2 °2θ, 21.0 °2θ, and 14.7 °2θ (±0.2 °2θ; ±0.1 °2θ; or ±0.0 °2θ; Cu Kα1 radiation).

In some embodiments, the solid form of Compound A tosylate Pattern 1 is crystalline Compound A tosylate Pattern 1 characterized by two or more, or three or more XRPD signals selected from the group consisting of 3.5 °2θ, 22.0 °2θ, 23.0 °2θ, 21.7 °2θ, 7.2 °2θ, 21.0 °2θ, 14.7 °2θ, 10.7 °2θ, 12.7 °2θ, and 18.4 °2θ (±0.2 °2θ; ±0.1 °2θ; or ±0.0 °2θ; Cu Kα1 radiation). In some embodiments, the solid form of Compound A tosylate Pattern 1 is crystalline Compound A tosylate Pattern 1 characterized by XRPD signals at 3.5 °2θ, 22.0 °2θ, 23.0 °2θ, 21.7 °2θ, 7.2 °2θ, 21.0 °2θ, 14.7 °2θ, 10.7 °2θ, 12.7 °2θ, and 18.4 °2θ (±0.2 °2θ; ±0.1 °2θ; or ±0.0 °2θ; Cu Kα1 radiation).

In some embodiments, the crystalline Compound A tosylate Pattern 1 is characterized by one, two, three, four, five, six, seven, eight, nine, ten, eleven, twelve, or thirteen XRPD signals selected from those set forth in Table 10.

TABLE 10
XRPD peak table of tosylate Pattern 1

| | | | | Rel. Int. |
|---|---|---|---|---|
| No. | Pos. [°2θ] | d-spacing [Å] | Height [cts] | [%] |
| 1 | 3.4707 | 25.45815 | 436.59 | 100 |
| 2 | 7.1537 | 12.35734 | 312.20 | 71.51 |
| 3 | 10.7124 | 8.25880 | 220.78 | 50.57 |
| 4 | 12.6695 | 6.98713 | 204.28 | 46.79 |
| 5 | 14.6897 | 6.03044 | 226.52 | 51.88 |
| 6 | 17.3482 | 5.11185 | 104.42 | 23.92 |
| 7 | 18.3591 | 4.83257 | 113.92 | 26.09 |
| 8 | 21.0208 | 4.22631 | 311.07 | 71.25 |
| 9 | 21.7151 | 4.09273 | 327.09 | 74.92 |
| 10 | 22.0225 | 4.03629 | 429.59 | 98.40 |
| 11 | 22.9548 | 3.87442 | 329.26 | 75.42 |
| 12 | 24.2064 | 3.67685 | 102.00 | 23.36 |
| 13 | 27.8589 | 3.20254 | 35.48 | 8.13 |

### Compound A Phosphate Pattern 1

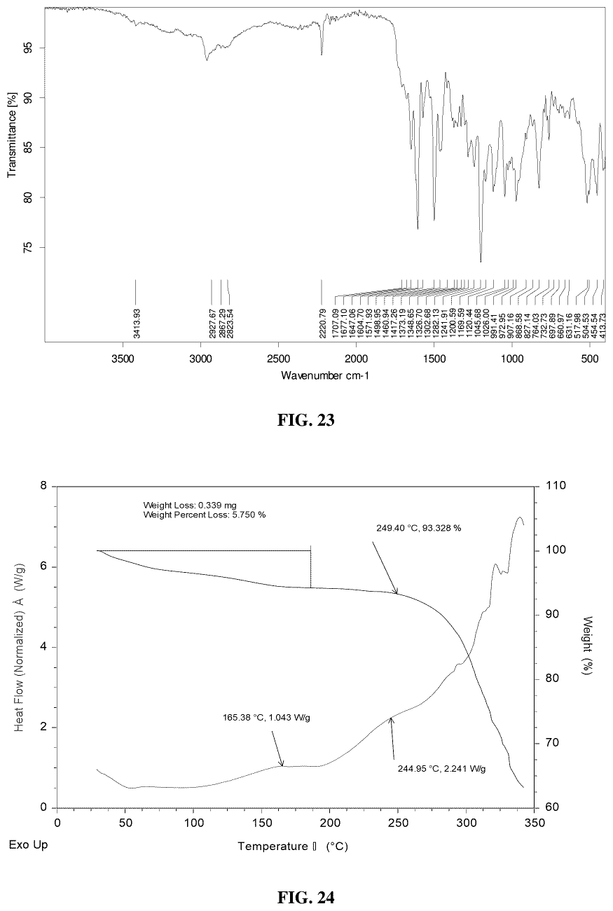

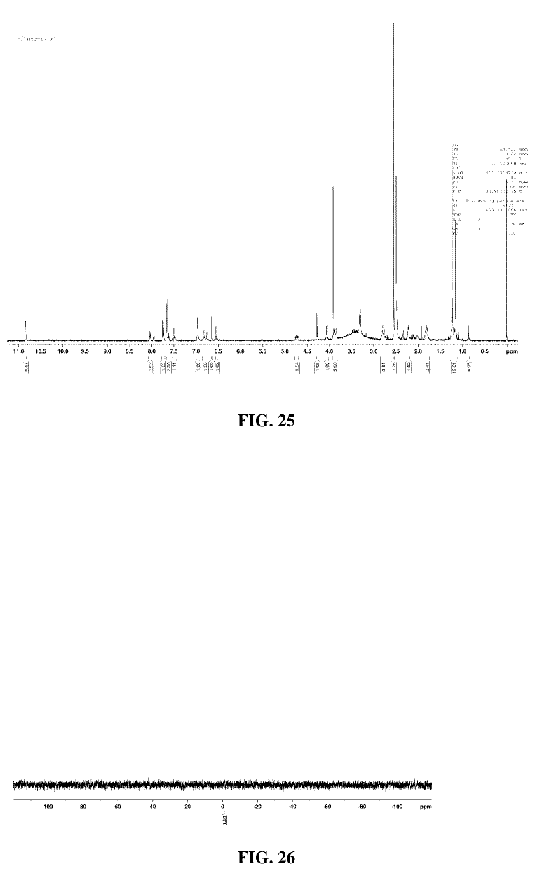

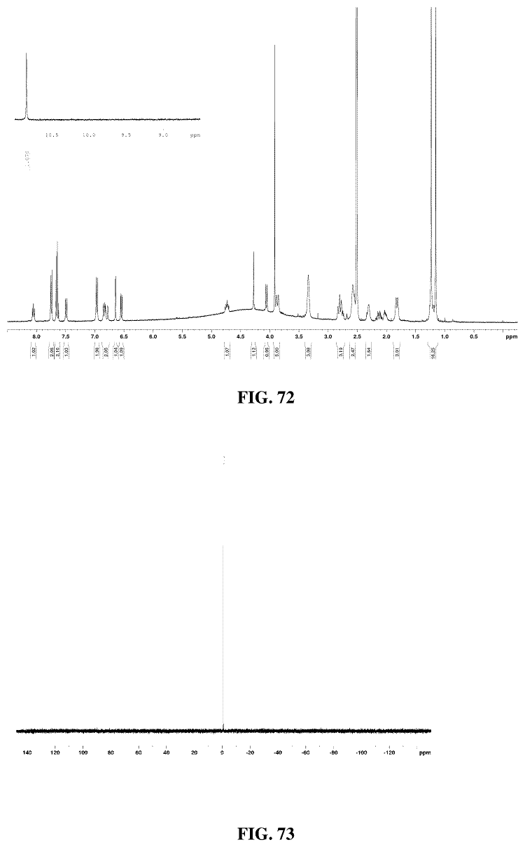

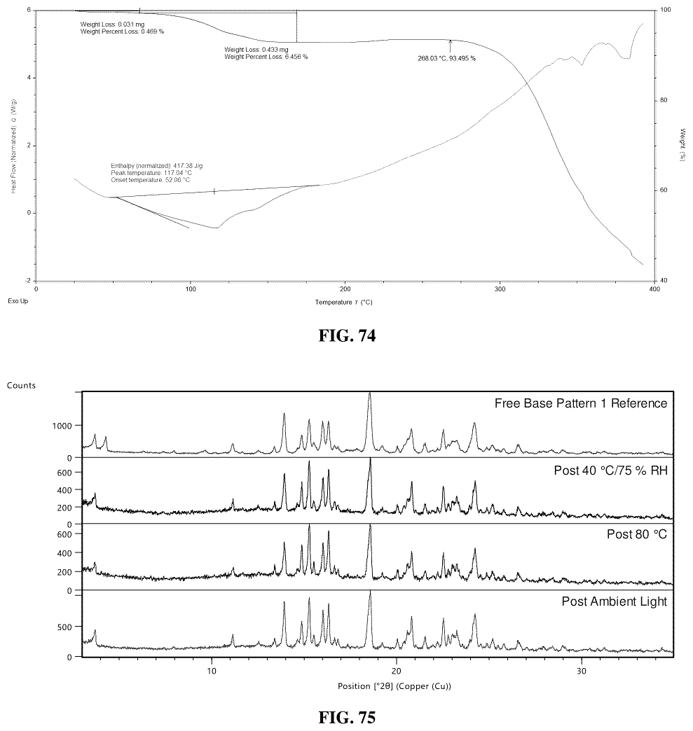

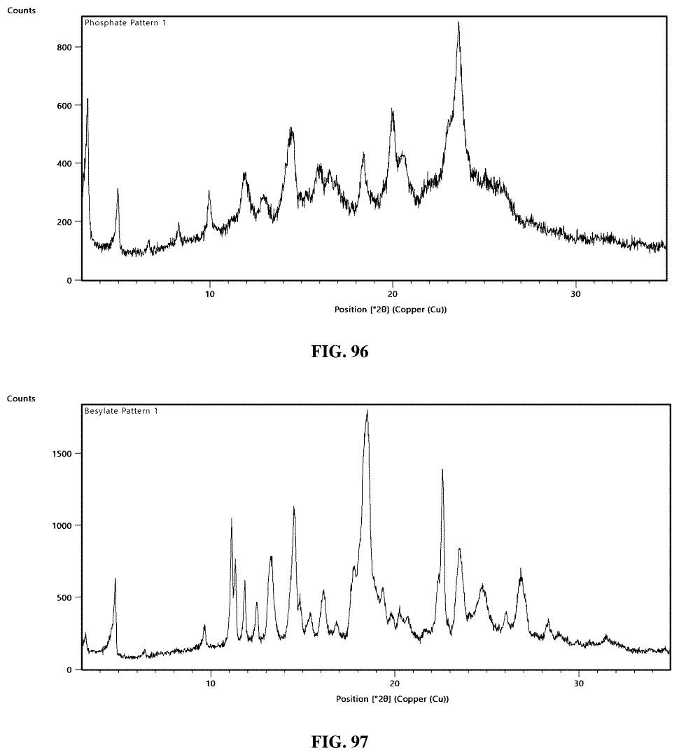

In some embodiments, the present disclosure provides solid forms of Compound A, e.g., crystalline forms of Compound A phosphate Pattern 1. In some embodiments, the Compound A phosphate Pattern 1 XRPD profile is substantially similar to that shown in FIG. 96. In some embodiments, the Compound A phosphate Pattern 1 FT-IR spectrum is substantially similar to that shown in FIG. 23. In some embodiments, the Compound A phosphate Pattern 1 ¹H NMR spectrum is substantially similar to that shown in FIG. 25 or FIG. 72. In some embodiments, the Compound A phosphate Pattern 1 ³¹P NMR spectrum is substantially similar to that shown in FIG. 26 or FIG. 73. In some embodiments, the Compound A phosphate Pattern 1 TGA profile is substantially similar to that shown in FIG. 24 or FIG. 74. In some embodiments, the Compound A phosphate Pattern 1 DSC profile is substantially similar to that shown in FIG. 24 or FIG. 74.

In some embodiments, the solid form of Compound A phosphate Pattern 1 is crystalline Compound A phosphate Pattern 1 characterized by two or three XRPD signals selected from the group consisting of 23.6 °2θ, 3.3 °2θ, and 19.9 °2θ (±0.2 °2θ; ±0.1 °2θ; or ±0.0 °2θ; Cu Kα1 radiation). In some embodiments, the solid form of Compound A phosphate Pattern 1 is crystalline Compound A phosphate Pattern 1 characterized by XRPD signals at 23.6 °2θ, 3.3 °2θ, and 19.9 °2θ (±0.2 °2θ; ±0.1 °2θ; or ±0.0 °2θ; Cu Kα1 radiation).

In some embodiments, the solid form of Compound A phosphate Pattern 1 is crystalline Compound A phosphate Pattern 1 characterized by two or more, or three or more XRPD signals selected from the group consisting of 23.6 °2θ, 3.3 °2θ, 19.9 °2θ, 14.6 °2θ, and 18.4 °2θ (±0.2 °2θ; ±0.1 °2θ; or ±0.0 °2θ; Cu Kα1 radiation). In some embodiments, the solid form of Compound A phosphate Pattern 1 is crystalline Compound A phosphate Pattern 1 characterized by XRPD signals at 23.6 °2θ, 3.3 °2θ, 19.9 °2θ, 14.6 °2θ, and 18.4 °2θ (±0.2 °2θ; ±0.1 °2θ; or ±0.0 °2θ; Cu Kα1 radiation).

In some embodiments, the solid form of Compound A phosphate Pattern 1 is crystalline Compound A phosphate Pattern 1 characterized by two or more, or three or more XRPD signals selected from the group consisting of 23.6 °2θ, 3.3 °2θ, 19.9 °2θ, 14.6 °2θ, 18.4 °2θ, 20.6 °2θ, and 16.1 °2θ (±0.2 °2θ; ±0.1 °2θ; or ±0.0 °2θ; Cu Kα1 radiation). In some embodiments, the solid form of Compound A phosphate Pattern 1 is crystalline Compound A phosphate Pattern 1 characterized by XRPD signals at 23.6 °2θ, 3.3 °2θ, 19.9 °2θ, 14.6 °2θ, 18.4 °2θ, 20.6 °2θ, and 16.1 °2θ (±0.2 °2θ; ±0.1 °2θ; or ±0.0 °2θ; Cu Kα1 radiation).

In some embodiments, the solid form of Compound A phosphate Pattern 1 is crystalline Compound A phosphate Pattern 1 characterized by two or more, or three or more XRPD signals selected from the group consisting of 23.6 °2θ, 3.3 °2θ, 19.9 °2θ, 14.6 °2θ, 18.4 °2θ, 20.6 °2θ, 16.1 °2θ, 11.9 °2θ, 5.0 °2θ, and 10.0 °2θ (±0.2 °2θ; ±0.1 °2θ; or ±0.0 °2θ; Cu Kα1 radiation). In some embodiments, the solid form of Compound A phosphate Pattern 1 is crystalline Compound A phosphate Pattern 1 characterized by XRPD signals at 23.6 °2θ, 3.3 °2θ, 19.9 °2θ, 14.6 °2θ, 18.4 °2θ, 20.6 °2θ, 16.1 °2θ, 11.9 °2θ, 5.0 °2θ, and 10.0 °2θ (±0.2 °2θ; ±0.1 °2θ; or ±0.0 °2θ; Cu Kα1 radiation).

In some embodiments, the crystalline Compound A phosphate Pattern 1 is characterized by one, two, three, four, five, six, seven, eight, nine, ten, eleven, twelve, thirteen, or fourteen XRPD signals selected from those set forth in Table 11.

TABLE 11
XRPD peak table of phosphate Pattern 1

| | | | | Rel. Int. |
|---|---|---|---|---|
| No. | Pos. [°2θ] | d-spacing [Å] | Height [cts] | [%] |
| 1 | 3.3199 | 26.61348 | 537.58 | 75.81 |
| 2 | 4.9733 | 17.76900 | 218.73 | 30.85 |
| 3 | 6.6513 | 13.28944 | 33.29 | 4.69 |
| 4 | 8.2888 | 10.66739 | 82.56 | 11.64 |
| 5 | 9.9769 | 8.86592 | 178.25 | 25.14 |
| 6 | 11.8812 | 7.44884 | 236.08 | 33.29 |
| 7 | 12.8688 | 6.87934 | 155.37 | 21.91 |
| 8 | 14.5722 | 6.07880 | 371.13 | 52.34 |
| 9 | 16.0609 | 5.51856 | 244.72 | 34.51 |
| 10 | 18.4101 | 4.81932 | 278.09 | 39.22 |
| 11 | 19.9435 | 4.45209 | 411.94 | 58.09 |
| 12 | 20.6004 | 4.31158 | 265.54 | 37.45 |
| 13 | 23.5976 | 3.77031 | 709.09 | 100 |
| 14 | 26.0194 | 3.42462 | 120.54 | 17.00 |

### Compound A Besylate Pattern 1

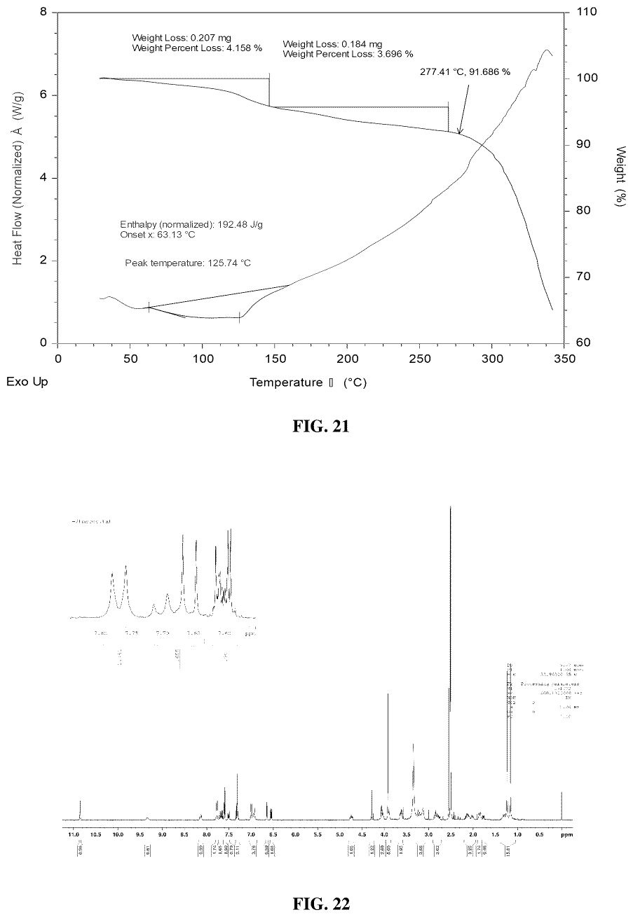

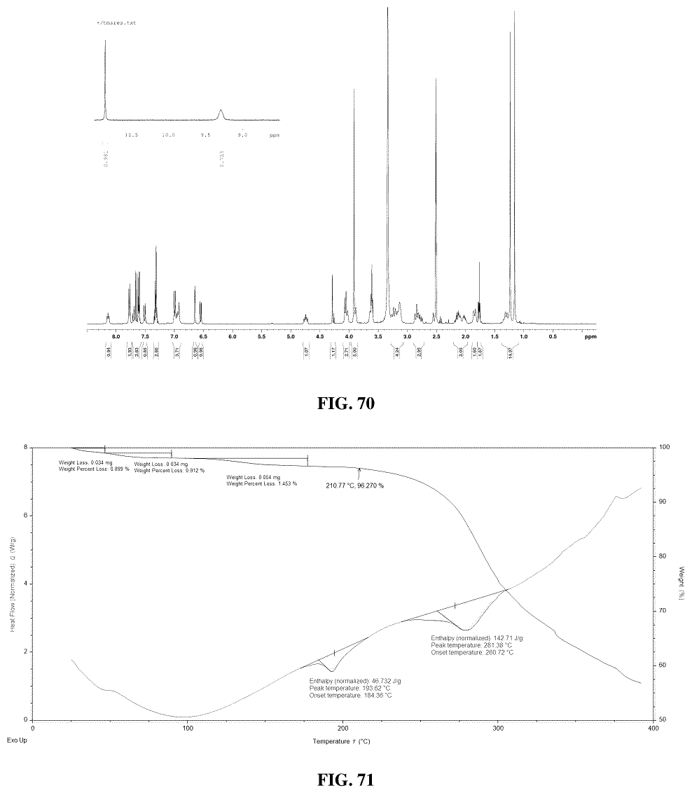

In some embodiments, the present disclosure provides solid forms of Compound A, e.g., crystalline forms of Compound A besylate Pattern 1. In some embodiments, the Compound A besylate Pattern 1 XRPD profile is substantially similar to that shown in FIG. 97. In some embodiments, the Compound A besylate Pattern 1 FT-IR spectrum is substantially similar to that shown in FIG. 20. In some embodiments, the Compound A besylate Pattern 1 ¹H NMR spectrum is substantially similar to that shown in FIG. 22 or FIG. 70. In some embodiments, the Compound A besylate Pattern 1 TGA profile is substantially similar to that shown in FIG. 21 or FIG. 71. In some embodiments, the Compound A besylate Pattern 1 DSC profile is substantially similar to that shown in FIG. 21 or FIG. 71.

In some embodiments, the solid form of Compound A besylate Pattern 1 is crystalline Compound A besylate Pattern 1 characterized by two or three XRPD signals selected from the group consisting of 18.5 °2θ, 18.3 °2θ, and 22.6 °2θ (±0.2 °2θ; ±0.1 °2θ; or ±0.0 °2θ; Cu Kα1 radiation). In some embodiments, the solid form of Compound A besylate Pattern 1 is crystalline Compound A besylate Pattern 1 characterized by XRPD signals at 18.5 °2θ, 18.3 °2θ, and 22.6 °2θ (±0.2 °2θ; ±0.1 °2θ; or ±0.0 °2θ; Cu Kα1 radiation).

In some embodiments, the solid form of Compound A besylate Pattern 1 is crystalline Compound A besylate Pattern 1 characterized by two or more, or three or more XRPD signals selected from the group consisting of 18.5 °2θ, 18.3 °2θ, 22.6 °2θ, 11.1 °2θ, and 14.6 °2θ (±0.2 °2θ; ±0.1 °2θ; or ±0.0 °2θ; Cu Kα1 radiation). In some embodiments, the solid form of Compound A besylate Pattern 1 is crystalline Compound A besylate Pattern 1 characterized by XRPD signals at 18.5 °2θ, 18.3 °2θ, 22.6 °2θ, 11.1 °2θ, and 14.6 °2θ (±0.2 °2θ; ±0.1 °2θ; or ±0.0 °2θ; Cu Kα1 radiation).

In some embodiments, the solid form of Compound A besylate Pattern 1 is crystalline Compound A besylate Pattern 1 characterized by two or more, or three or more XRPD signals selected from the group consisting of 18.5 °2θ, 18.3 °2θ, 22.6 °2θ, 11.1 °2θ, 14.6 °2θ, 23.5 °2θ, and 13.3 °2θ (±0.2 °2θ; ±0.1 °2θ; or ±0.0 °2θ; Cu Kα1 radiation). In some embodiments, the solid form of Compound A besylate Pattern 1 is crystalline Compound A besylate Pattern 1 characterized by XRPD signals at 18.5 °2θ, 18.3 °2θ, 22.6 °2θ, 11.1 °2θ, 14.6 °2θ, 23.5 °2θ, and 13.3 °2θ (±0.2 °2θ; ±0.1 °2θ; or ±0.0 °2θ; Cu Kα1 radiation).

In some embodiments, the solid form of Compound A besylate Pattern 1 is crystalline Compound A besylate Pattern 1 characterized by two or more, or three or more XRPD signals selected from the group consisting of 18.5 °2θ, 18.3 °2θ, 22.6 °2θ, 11.1 °2θ, 14.6 °2θ, 23.5 °2θ, 13.3 °2θ, 11.3 °2θ, 17.8 °2θ, and 4.8 °2θ (±0.2 °2θ; ±0.1 °2θ; or ±0.0 °2θ; Cu Kα1 radiation). In some embodiments, the solid form of Compound A besylate Pattern 1 is crystalline Compound A besylate Pattern 1 characterized by XRPD signals at 18.5 °2θ, 18.3 °2θ, 22.6 °2θ, 11.1 °2θ, 14.6 °2θ, 23.5 °2θ, 13.3 °2θ, 11.3 °2θ, 17.8 °2θ, and 4.8 °2θ (±0.2 °2θ; ±0.1 °2θ; or ±0.0 °2θ; Cu Kα1 radiation).

In some embodiments, the crystalline Compound A besylate Pattern 1 is characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty XRPD signals selected from those set forth in Table 12.

TABLE 12
XRPD peak table of besylate Pattern 1

| | | | | Rel. Int. |
|---|---|---|---|---|
| No. | Pos. [°2θ] | d-spacing [Å] | Height [cts] | [%] |
| 1 | 3.2079 | 27.54304 | 165.64 | 10.55 |
| 2 | 4.8192 | 18.33697 | 559.47 | 35.65 |
| 3 | 6.3869 | 13.83909 | 31.40 | 2.00 |
| 4 | 9.6657 | 9.15070 | 163.56 | 10.42 |
| 5 | 11.1338 | 7.94716 | 936.62 | 59.68 |
| 6 | 11.3464 | 7.79870 | 607.64 | 38.72 |
| 7 | 11.8678 | 7.45723 | 462.85 | 29.49 |
| 8 | 12.4881 | 7.08821 | 339.69 | 21.64 |
| 9 | 13.1324 | 6.74183 | 508.82 | 32.42 |
| 10 | 13.3327 | 6.64101 | 619.35 | 39.46 |
| 11 | 14.5689 | 6.08018 | 929.90 | 59.25 |
| 12 | 14.8502 | 5.96561 | 349.60 | 22.28 |
| 13 | 15.4179 | 5.74723 | 245.36 | 15.63 |
| 14 | 16.1567 | 5.48604 | 383.37 | 24.43 |
| 15 | 16.7843 | 5.28228 | 164.80 | 10.50 |
| 16 | 17.7518 | 4.99651 | 570.62 | 36.36 |
| 17 | 18.2687 | 4.85629 | 1306.02 | 83.22 |
| 18 | 18.5421 | 4.78529 | 1569.44 | 100 |
| 19 | 19.3548 | 4.58616 | 423.95 | 27.01 |
| 20 | 19.8401 | 4.47507 | 220.93 | 14.08 |
| 21 | 20.2426 | 4.38699 | 250.20 | 15.94 |
| 22 | 20.7061 | 4.28981 | 207.10 | 13.20 |
| 23 | 22.3403 | 3.97958 | 460.24 | 29.33 |
| 24 | 22.5739 | 3.93893 | 1209.04 | 77.04 |
| 25 | 23.4851 | 3.78811 | 679.64 | 43.30 |
| 26 | 24.7073 | 3.60343 | 407.06 | 25.94 |
| 27 | 26.0343 | 3.42269 | 226.70 | 14.44 |
| 28 | 26.8719 | 3.31788 | 457.13 | 29.13 |
| 29 | 28.2913 | 3.15456 | 137.28 | 8.75 |
| 30 | 31.4664 | 2.84313 | 65.23 | 4.16 |

### Methods of Ubiquitinating/Degrading a Target Protein in a Cell

The present disclosure provides a method of ubiquitinating/degrading a target protein in a cell.

In some embodiments, the method comprises administering a solid form of Compound A of the disclosure or a salt form of Compound A of the disclosure, wherein Compound A is a bifunctional compound comprising an E3 ubiquitin ligase binding moiety and a protein targeting moiety linked via a linker moiety.

In some embodiments, the E3 ubiquitin ligase binding moiety is coupled to the protein targeting moiety and wherein the E3 ubiquitin ligase binding moiety recognizes a ubiquitin pathway protein (e.g., an ubiquitin ligase, preferably an E3 ubiquitin ligase) and the protein targeting moiety recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

In some embodiments, this application provides a solid form of Compound A of the disclosure or a salt form of Compound A of the disclosure that degrades the androgen receptor (AR) protein.

In some embodiments, the present disclosure is directed to a method of treating a patient in need for a disease state or condition modulated through a protein where the degradation of that protein will produce a therapeutic effect in that patient, the method comprising administering to a patient in need an effective amount of a solid form of Compound A of the disclosure or a salt form of Compound A of the disclosure, optionally in combination with another anti-cancer agent. The disease state or condition may be a disease caused by overexpression of a protein, which leads to a disease state and/or condition.

### Methods of Treatment

In one aspect, the present application pertains to a method of treating and/or preventing cancer comprising administering to a subject in need thereof a therapeutically effective amount of a solid form of Compound A of the disclosure or a salt form of Compound A of the disclosure.

In one aspect, the present application pertains to a method of treating and/or preventing cancer comprising administering to a subject in need thereof a therapeutically effective amount of a solid form of Compound A of the disclosure or a salt form of Compound A of the disclosure, in combination with one or more additional anti-cancer agents.

The methods of treating cancer described herein result in a reduction in tumor size. Alternatively, or in addition, the cancer is metastatic cancer and this method of treatment includes inhibition of metastatic cancer cell invasion.

In some embodiments, the cancer is prostate cancer.

In some embodiments, the cancer is metastatic prostate cancer.

In some embodiments, the cancer is castrate-resistant prostate cancer.

In some embodiments, the cancer is metastatic castrate-resistant prostate cancer (mCRPC).

In some embodiments, the prostate cancer is castrate-sensitive prostate cancer.

In some embodiments, the prostate cancer is metastatic castrate-sensitive prostate cancer.

In some embodiments, the prostate cancer is prostate cancer naïve to novel hormonal agents (NHA).

In some embodiments, the prostate cancer is metastatic prostate cancer naïve to novel hormonal agents (NHA).

In some embodiments, the prostate cancer is castrate-resistant prostate cancer naïve to novel hormonal agents (NHA).

In some embodiments, the prostate cancer is castrate-sensitive prostate cancer naïve to novel hormonal agents (NHA).

In some embodiments, the prostate cancer is metastatic castrate-resistant prostate cancer naïve to novel hormonal agents (NHA).

In some embodiments, the prostate cancer is metastatic castrate-sensitive prostate cancer naïve to novel hormonal agents (NHA).

In some embodiments, the prostate cancer is not prostate cancer naïve to novel hormonal agents (NHA). In some embodiments, the prostate cancer that is not prostate cancer naïve to novel hormonal agents (NHA) is also metastatic prostate cancer, castrate-resistant prostate cancer, metastatic castrate-resistant prostate cancer, castrate-sensitive prostate cancer, or metastatic castrate-sensitive prostate cancer.

In one aspect, the application pertains to treating prostate cancer with a solid form of Compound A of the disclosure or a salt form of Compound A of the disclosure in combination with another anti-cancer agent. In some embodiments, the prostate cancer treated with the combination of a solid form of Compound A of the disclosure or a salt form of Compound A of the disclosure and another anti-cancer agent is metastatic prostate cancer, castrate-resistant prostate cancer, metastatic castrate-resistant prostate cancer (mCRPC), castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA).

In some embodiments, the prostate cancer treated with the combination of a solid form of Compound A of the disclosure or a salt form of Compound A of the disclosure and another anti-cancer agent is not prostate cancer naïve to novel hormonal agents (NHA). In some embodiments, the prostate cancer that is not prostate cancer naïve to novel hormonal agents (NHA) is also metastatic prostate cancer, castrate-resistant prostate cancer, metastatic castrate-resistant prostate cancer, castrate-sensitive prostate cancer, or metastatic castrate-sensitive prostate cancer.

In some embodiments, the other anti-cancer agent is abiraterone, estramustine, docetaxel, ketoconazole, goserelin, histrelin, triptorelin, buserelin, cyproterone, flutamide, bicalutamide, nilutamide, pamidronate, zolendronate, or a pharmaceutically acceptable salt thereof.

In some embodiments, treating cancer results in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In some embodiments, treating cancer results in a reduction in tumor volume. Preferably, after treatment, tumor volume is reduced by 5% or greater relative to its volume prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In some embodiments, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In some embodiments, the specified magnification is 2×, 3×, 4×, 5×, 10×, or 50×.

In some embodiments, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In some embodiments, the number of metastatic lesions may be measured by counting metastatic lesions visible to the naked eye or at a specified magnification. In some embodiments, the specified magnification is 2×, 3×, 4×, 5×, 10×, or 50×.

In some embodiments, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In some embodiments, an increase in average survival time of a population may be measured, for example, by calculating for a population the average length of survival following initiation of treatment with an active agent or compound of the disclosure. In some embodiments, an increase in average survival time of a population may also be measured, for example, by calculating for a population the average length of survival following completion of a first round of treatment with an active agent or compound of the disclosure.

In some embodiments, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In some embodiments, an increase in average survival time of a population may be measured by calculating for a population the average length of survival following initiation of treatment with an active agent or compound of the disclosure. In some embodiments, an increase in average survival time of a population may be measured by calculating for a population the average length of survival following completion of a first round of treatment with a compound of the disclosure.

In some embodiments, treating cancer results in a decrease in tumor growth rate. Preferably, after treatment, tumor growth rate is reduced by at least 5% relative to growth rate prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In some embodiments, tumor growth rate is measured according to a change in tumor diameter per unit time.

In some embodiments, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In some embodiments, tumor regrowth is measured by measuring an increase in the diameter of a tumor after a prior tumor shrinkage that followed treatment. In some embodiments, a decrease in tumor regrowth is indicated by failure of tumors to reoccur after treatment has stopped.

The dosages of the solid forms of Compound A of the disclosure or salt forms of Compound A of the disclosure for any of the methods and uses described herein vary depending on the agent, the age, weight, and clinical condition of the recipient subject, and the experience and judgment of the clinician or practitioner administering the therapy, among other factors affecting the selected dosage.

The therapeutically effective amount of the solid form of Compound A of the disclosure or the salt form of Compound A of the disclosure may be administered one or more times over a day for up to 30 or more days, followed by 1 or more days of non-administration of the compound. This type of treatment schedule, i.e., administration of the solid form of Compound A of the disclosure or the salt form of Compound A of the disclosure on consecutive days followed by non-administration of solid/salt forms on consecutive days may be referred to as a treatment cycle. A treatment cycle may be repeated as many times as necessary to achieve the intended affect.

In some embodiments, the therapeutically effective amount of the solid form of Compound A of the disclosure or the salt form of Compound A of the disclosure is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, twenty, twenty-five, thirty consecutive days, or, once, twice, three times, four times, or more daily, in single or divided doses, for 2 months, 3 months, 4 months, 5 months, 6 months, or longer.

In some embodiments, the therapeutically effective amount of the solid form of Compound A of the disclosure or the salt form of Compound A of the disclosure is about 10 to about 40 mg, about 20 to about 50 mg, about 30 to about 60 mg, about 40 to about 70 mg, about 50 to about 80 mg, about 60 to about 90 mg, about 70 to about 100 mg, about 80 to about 110 mg, about 90 to about 120 mg, about 100 to about 130 mg, about 110 to about 140 mg, about 120 to about 150 mg, about 130 to about 160 mg, about 140 to about 170 mg, about 150 to about 180 mg, about 160 to about 190 mg, about 170 to about 200 mg, about 180 to about 210 mg, about 190 to about 220 mg, about 200 to about 230 mg, about 210 to about 240 mg, about 220 to about 250 mg, about 230 to about 260 mg, about 240 to about 270 mg, about 250 to about 280 mg, about 260 to about 290 mg, about 270 to about 300 mg, about 280 to about 310 mg, about 290 to about 320 mg, about 300 to about 330 mg, about 310 to about 340 mg, about 320 to about 350 mg, about 330 to about 360 mg, about 340 to about 370 mg, about 350 to about 380 mg, about 360 to about 390 mg, about 370 to about 400 mg, about 380 to about 410 mg, about 390 to about 420 mg, about 400 to about 430 mg, about 410 to about 440 mg, about 420 to about 450 mg, about 430 to about 460 mg, about 440 to about 470 mg, about 450 to about 480 mg, about 460 to about 490 mg, about 470 to about 500 mg, about 480 to about 510 mg, about 490 to about 520 mg, about 500 to about 530 mg, about 510 to about 540 mg, about 520 to about 550 mg, about 530 to about 560 mg, about 540 to about 570 mg, about 550 to about 580 mg, about 560 to about 590 mg, about 570 to about 600 mg, about 580 to about 610 mg, about 590 to about 620 mg, about 600 to about 630 mg, about 610 to about 640 mg, about 620 to about 650 mg, about 630 to about 660 mg, about 640 to about 670 mg, about 650 to about 680 mg, about 660 to about 690 mg, about 670 to about 700 mg, about 680 to about 710 mg, about 690 to about 720 mg, about 700 to about 730 mg, about 710 to about 740 mg, about 720 to about 750 mg, about 730 to about 760 mg, about 740 to about 770 mg, about 750 to about 780 mg, about 760 to about 790 mg, about 770 to about 800 mg, about 780 to about 810 mg, about 790 to about 820 mg, about 800 to about 830 mg, about 810 to about 840 mg, about 820 to about 850 mg, about 830 to about 860 mg, about 840 to about 870 mg, about 850 to about 880 mg, about 860 to about 890 mg, about 870 to about 900 mg, about 880 to about 910 mg, about 890 to about 920 mg, about 900 to about 930 mg, about 910 to about 940 mg, about 920 to about 950 mg, about 930 to about 960 mg, about 940 to about 970 mg, about 950 to about 980 mg, about 960 to about 990 mg, or about 970 to about 1,000 mg administered once, twice, three times, four times, or more daily in single or divided doses (which dose may be adjusted for the patient's weight in kg, body surface area in m², and/or age in years).

In some embodiments, the therapeutically effective amount of the solid form of Compound A of the disclosure or the salt form of Compound A of the disclosure is about 70 mg to about 1000 mg administered once, twice, three times, four times, or more daily in single or divided doses (which dose may be adjusted for the patient's weight in kg, body surface area in m², and/or age in years).

In some embodiments, the therapeutically effective amount of the solid form of Compound A of the disclosure or the salt form of Compound A of the disclosure is about 70 mg, 100 mg, 105 mg, 140 mg, 150 mg, 175 mg, 210 mg, 245 mg, 280 mg, 300 mg, 315 mg, 350 mg, 385 mg, 420 mg, 455 mg, 490 mg, 525 mg, 560 mg, 595 mg, 630 mg, 665 mg, or 700 mg administered once, twice, three times, four times, or more daily in single or divided doses (which dose may be adjusted for the patient's weight in kg, body surface area in m², and/or age in years).

In some embodiments, the therapeutically effective amount of the solid form of Compound A of the disclosure or the salt form of Compound A of the disclosure is administered to the subject once daily. In some embodiments, this daily dose of the solid form of Compound A of the disclosure or the salt form of Compound A of the disclosure may be administered to the subject all at once. In some embodiments, this daily dose of the solid form of Compound A of the disclosure or the salt form of Compound A of the disclosure may be administered to the subject in two portions (i.e., a divided dose). In some embodiments, this daily dose of the solid form of Compound A of the disclosure or the salt form of Compound A of the disclosure may be administered to the subject in three divided doses. In some embodiments, this daily dose of the solid form of Compound A of the disclosure or the salt form of Compound A of the disclosure may be administered to the subject in four divided doses. In some embodiments, this daily dose of the solid form of Compound A of the disclosure or the salt form of Compound A of the disclosure may be administered to the subject in five or more divided doses. In some embodiments, these portions or divided doses are administered to the subject at regular intervals throughout the day, for example, every 12 hours, every 8 hours, every 6 hours, every 5 hours, every 4 hours, etc.

The therapeutically effective amount of the solid form of Compound A of the disclosure or the salt form of Compound A of the disclosure can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the solid form of Compound A of the disclosure or the salt form of Compound A of the disclosure or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, once every two weeks, or monthly depending on half-life and clearance rate of the particular formulation.

In some embodiments, for the methods of treating prostate cancer with the combination of the solid form of Compound A of the disclosure or the salt form of Compound A of the disclosure and another anti-cancer agent, the therapeutically effective amount of the solid form of Compound A of the disclosure or the salt form of Compound A of the disclosure is described herein, and the therapeutically effective amount of the other anti-cancer agent is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, or thirty consecutive days, or, once, twice, three times, four times, or more daily, in single or divided doses, for 2 months, 3 months, 4 months, 5 months, 6 months, or longer.

In some embodiments, the solid form of Compound A of the disclosure or the salt form of Compound A of the disclosure and the other anti-cancer agent are administered to the subject simultaneously. In some embodiments, the solid form of Compound A of the disclosure or the salt form of Compound A of the disclosure and the other anti-cancer agent are administered to the subject sequentially.

In some embodiments, the solid form of Compound A of the disclosure or the salt form of Compound A of the disclosure and the other anti-cancer agent are administered to the subject in temporal proximity.

In some embodiments, “temporal proximity” means that administration of the solid form of Compound A of the disclosure or the salt form of Compound A of the disclosure occurs within a time period before or after the administration of additional anti-cancer agent, such that the therapeutic effect of the solid form of Compound A of the disclosure or the salt form of Compound A of the disclosure overlaps with the therapeutic effect of the additional anti-cancer agent. In some embodiments, the therapeutic effect of the solid form of Compound A of the disclosure or the salt form of Compound A of the disclosure completely overlaps with the therapeutic effect of the additional anti-cancer agent. In some embodiments, “temporal proximity” means that administration of the solid form of Compound A of the disclosure or the salt form of Compound A of the disclosure occurs within a time period before or after the administration of additional anti-cancer agent, such that there is a synergistic effect between the solid form of Compound A of the disclosure or the salt form of Compound A of the disclosure and the anti-cancer agent.

“Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

### EXAMPLES

### Example 1. General Methods of Analysis

### X-Ray Powder Diffraction (XRPD)

XRPD analysis was carried out on a PANalytical X'pert pro with PIXcel detector (128 channels), scanning the samples between 3 and 35 °2θ. The material was gently ground to release any agglomerates and loaded onto a multi-well plate with Kapton or Mylar polymer film to support the sample. The multi-well plate was then placed into the diffractometer and analyzed using Cu K radiation (α₁γ=1.54060 Å; α₂=1.54443 Å; β=1.39225 Å; α₁:α₂ ratio=0.5) running in transmission mode (step size 0.0130 °2θ, step time 18.87 s) using 40 kV/40 mA generator settings. Data were visualized and images generated using the HighScore Plus 4.7 desktop application (PANalytical, 2θ17).

### Spinning Stage X-Ray Powder Diffraction (XRPD)

XRPD analysis of the second set of competitive slurry experiments was carried out on a Philips X'Pert Pro Multipurpose diffractometer equipped with a spinning stage autosampler. The samples were scanned between 5 and 34.997 °2θ using Cu K radiation (α₁γ=1.54060 Å; α₂=1.54443 Å; β=1.39225 Å; α₁:α₂ ratio=0.5) running in Bragg-Brentano geometry (step size 0.008 °2θ, step time 10.160 s, rotation period 2 s) using 40 kV/40 mA generator settings. Data were visualized and images generated using the HighScore Plus 4.7 desktop application (PANalytical, 2θ17).

### Polarised Light Microscopy (PLM)

The presence of crystallinity (birefringence) was determined using an Olympus BX50 microscope, equipped with cross-polarising lenses and a Motic camera. Images were captured using Motic Images Plus 2.0. All images were recorded using the 20× objective, unless otherwise stated.

### Thermogravimetric/Differential Thermal Analysis (TG/DTA)

Approximately 5 mg of material was weighed into an open aluminium pan and loaded into a simultaneous thermogravimetric/differential thermal analyser (TG/DTA) and held at room temperature. The sample was then heated at a rate of 10° C./min from 20° C. to 300° C. during which time the change in sample weight was recorded along with any differential thermal events (DTA). Nitrogen was used as the purge gas, at a flow rate of 300 cm³/min.

### Differential Scanning Calorimetry (DSC)

Approximately 5 mg of material was weighed into an aluminium DSC pan and sealed non-hermetically with a pierced aluminium lid. The sample pan was then loaded into a TA Instruments Discovery DSC 2500 differential scanning calorimeter equipped with a RC90 cooler. The sample and reference were heated to 270° C. at a scan rate of 10° C./min and the resulting heat flow response monitored. The sample was re-cooled to 20° C. and then reheated again to 270° C. all at 10° C./min. Nitrogen was used as the purge gas, at a flow rate of 50 cm³/min.

### Infrared Spectroscopy (IR)

Infrared spectroscopy was carried out on a Bruker ALPHA P spectrometer. Sufficient material was placed onto the centre of the plate of the spectrometer and the spectra were obtained using the following parameters:
Resolution: 4 cm⁻¹
Background Scan Time: 16 scans
Sample Scan Time: 16 scans
Data Collection: 4000 to 400 cm⁻¹
Result Spectrum: Transmittance
Software: OPUS version 6

### Nuclear Magnetic Resonance (NMR)

NMR experiments were performed on a Bruker A VIIIHD spectrometer operating at 400 MHZ for protons. Experiments were performed in deuterated DMSO and each sample was prepared to ca. 10 mM concentration.

### Dynamic Vapour Sorption (DVS)

10-20 mg of sample was placed into a mesh vapour sorption balance pan and loaded into a DVS-1 dynamic vapour sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis was then carried out on any solid retained.

### Variable Temperature X-Ray Powder Diffraction (VT-XRPD)

VT-XRPD analysis was carried out on a Philips X'Pert Pro Multipurpose diffractometer equipped with a temperature chamber. The samples were scanned between 4 and 35.99 °2θ using Cu K radiation (α₁γ=1.54060 Å; α₂=1.54443 Å; β=1.39225 Å; α₁:α₂ ratio=0.5) running in Bragg-Brentano geometry (step size 0.008 °2θ) using 40 kV/40 mA generator settings. The sample was heated at a 10° C./min heating rate and held at each temperature for 3 minutes before XRPD analysis. Measurements were performed at 25° C., 164° C., 180° C., 203° C., 216° C., 234° C., 250° C. and again at 25° C.

### High Performance Liquid Chromatography-Ultraviolet Detection (HPLC-UV)

Instrument: Waters H-Class UPLC
Column: ACQUITY UPLC BEH C18 1.7 μm 2.1×50 mm (Part No. 186002350
Column Temperature: 45° C.
Autosampler Temperature: Ambient
UV wavelength: 254 nm
Injection Volume: 2 μL
Flow Rate: 0.8 mL/min
Mobile Phase A: 0.1% TFA in water
Mobile Phase B: 0.1% TFA in acetonitrile
Diluent: 0.1% TFA in water:acetonitrile (50:50% v/v)
Gradient program:

| Time (minutes) | Solvent B [%] |
|---|---|
| 0 | 5 |
| 0.5 | 5 |
| 9.20 | 99 |
| 9.50 | 99 |
| 9.55 | 5 |
| 15.0 | 5 |

### Example 2. Characterization of Compound A

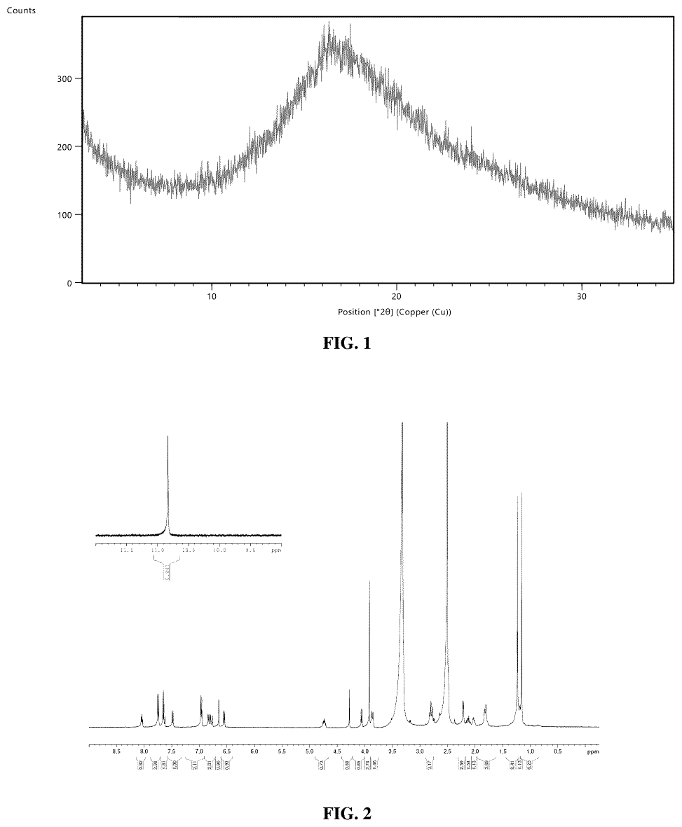

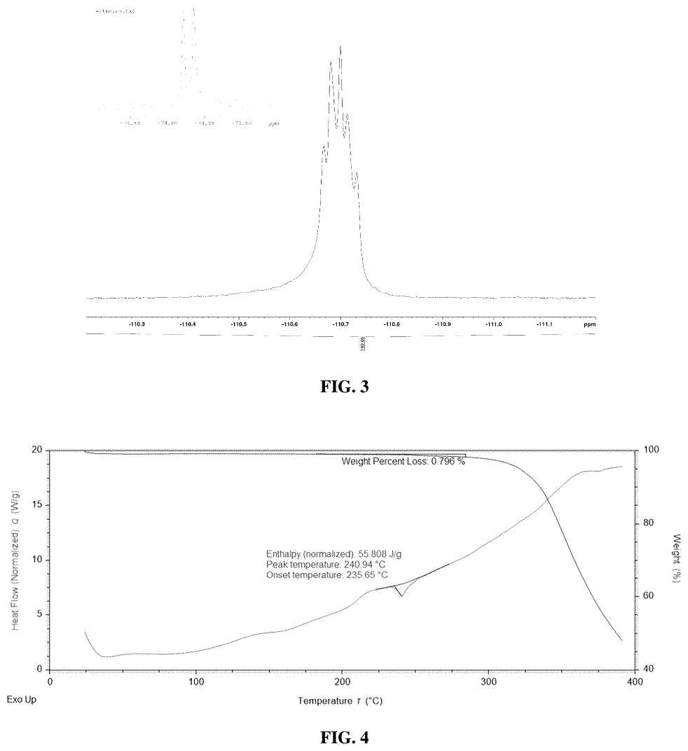

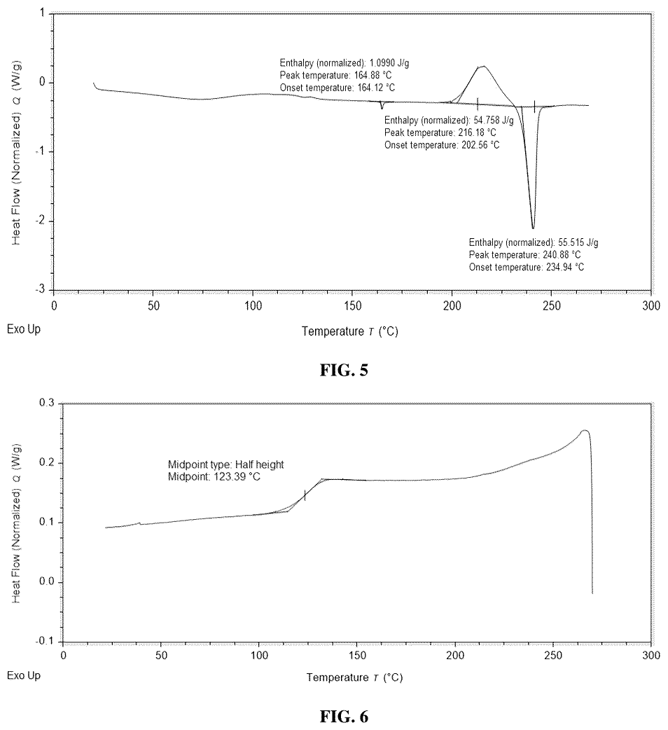

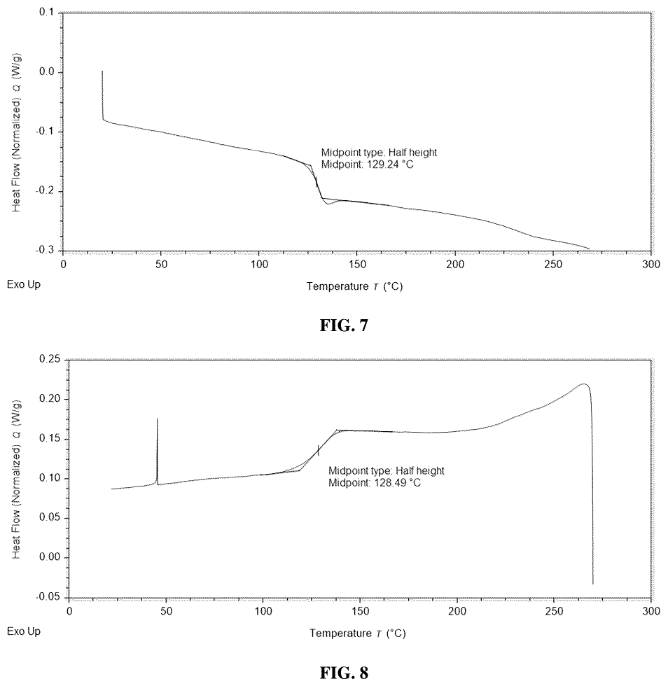

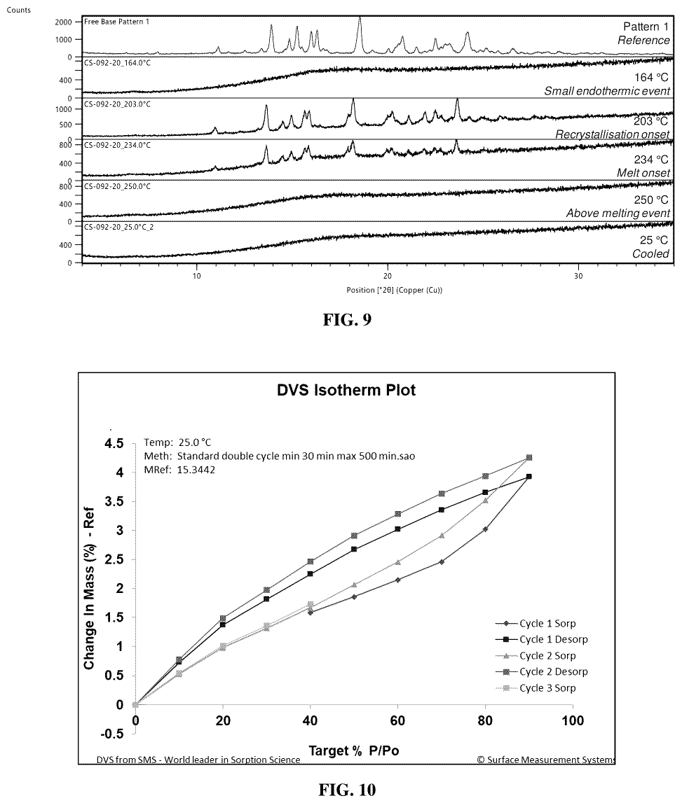

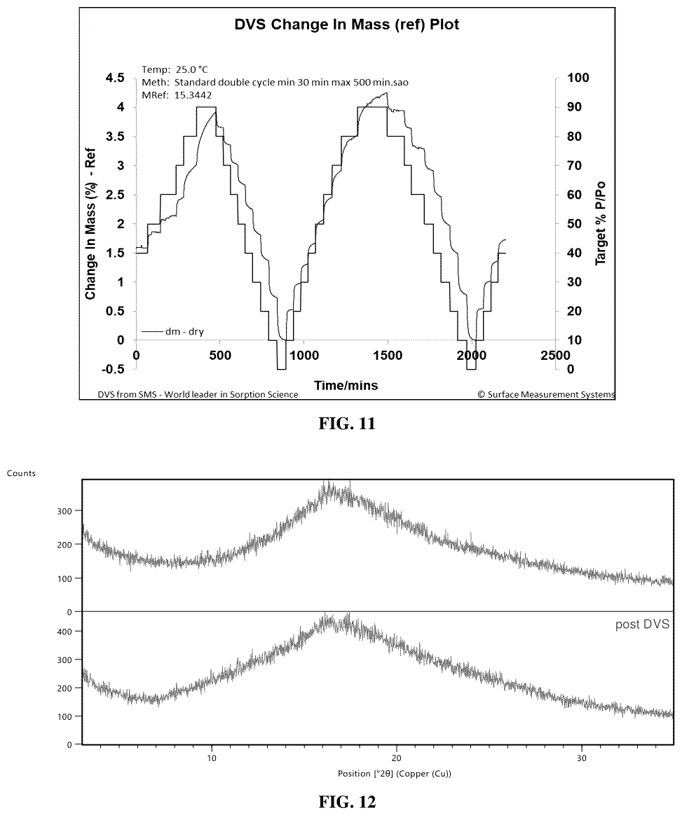

Compound A was characterized by the following techniques: XRPD, PLM, Multinuclear NMR, TG/DSC, VT-XRPD, DSC, DSV, and HPLC.
Initial characterization of Compound A determined the material was amorphous by XRPD, see FIG. 1. This was confirmed by PLM where no birefringent solids were observed. The particles were ca. 5 μm in size with a plate-like morphology.
Multinuclear NMR (¹H NMR, C HSQC and ¹⁹F) found the data to be consistent with the structure for Compound A. There was a ca. 1% impurity peak present in the ¹⁹F spectrum at −74.5 ppm, likely due to TFA. See FIGS. 2 and 3.
TG/DSC analysis found a mass loss of 0.8 wt. % (0.36 eq.) water at the start of the experiment in the TG trace of the TG/DSC, likely due to surface moisture. Decomposition was noted above 300° C. In the DSC trace, an endothermic, melting event was observed with an onset of 236° C. See FIG. 4.
DSC analysis elucidated three events in the first heat. A small endothermic event with an onset of 164° C., likely caused by the melting of small amounts of crystalline material in the bulk sample. A broad exothermic event with an onset of 203° C. due to recrystallization of the material followed by a sharp endothermic event with an onset of 235° C. due to melting. See FIG. 5. This data corresponds well with the data obtained in the TG/DSC. Glass transitions were observed in the cooling cycle, second heating cycle, and second cooling cycle with midpoints at 123° C., 129° C., and 128° C. respectively. See FIGS. 6-8.
VT-XRPD analysis determined that when Compound A was heated to 164° C., the temperature at which a small endothermic event was observed by DSC, no changes in crystallinity were noted. When heated to the onset of the exothermic event observed by DSC (203° C.), recrystallisation to Pattern 1 was noted-slightly shifted compared to reference diffractogram collected in transmission mode. When pattern 1 was heated to the endothermic event (thought to be due to melting) observed by DSC, a slight reduction in crystallinity was noted. When heated further, melting was observed. No recrystallisation was evident on cooling. See FIG. 9.
DVS analysis determined that the material was hygroscopic with an average mass uptake of 4.08 wt. % (1.91 eq.) of water at 90% RH, see FIG. 10 for the isotherm plot and FIG. 11 for the kinetic plot. Crystallization was not observed by XRPD post DVS, see FIG. 12.

TABLE 13
Summary of the initial characterisation

| | | Compound A |
|---|---|---|
| XRPD | | Material was amorphous |
| PLM | | Non birefringent solids ca. 5 μm with plate-like morphology |
| NMR | | Consistent with structure, 1% impurity in 19F |
| TG/DSC | TG trace | 0.8 wt. % mass loss, decomposition from 300° C. |
| | DSC | Melt with onset at 236° C., peak at 241° C. |
| | trace | |
| DSC | First | Small endothermic event: onset 164° C. (melt of trace crystalline material), |
| | heat | exothermic event: onset 203° C. (crystallization), endothermic event: onset |
| | | 235° C. (melt that corresponds to TG/DSC) |
| | First cool | Glass transition with midpoint at 123° C. |
| | Second | Glass transition with midpoint at 129° C. |
| | heat | |
| | Second | Glass transition with midpoint at 128° C. |
| | cool | |

| | |
|---|---|
| DVS | 4.08 wt. % (1.91 eq. water) at 90% RH - Material is hygroscopic and |
| | remained amorphous by XRPD |
| HPLC | 97.4% pure by relative area |

### Example 3. Solvent Solubility Screen of Compound A

A known volume aliquot (typically 5 volumes) of solvent was added to approximately 5 mg of Compound A. See Table 14 for the selected solvents for the solvent solubility screen.

TABLE 14
Selected solvents used in the solvent solubility screen

| Number | Solvent System | ICH Class |
|---|---|---|
| 1 | 1-Butanol | 3 |
| 2 | 1-Propanol | 3 |
| 3 | 1,4-Dioxane | 2 |
| 4 | 2-Butanone (MEK) | 3 |
| 5 | 2-Ethoxyethanol | 2 |
| 6 | 2-Methyl THF | Not classified |
| 7 | 2-Methyl-1-propanol | 3 |
| 8 | 2-Propanol | 3 |
| 9 | Acetone | 3 |
| 10 | Acetonitrile | 2 |
| 11 | Anisole | 3 |
| 12 | tert-Butylmethyl ether | 3 |
| 13 | Butyl acetate | 3 |
| 14 | Dichloromethane | 2 |
| 15 | Dimethylsulfoxide | 3 |
| 16 | Ethanol | 3 |
| 17 | Ethyl acetate | 3 |
| 18 | Ethyl ether | 3 |
| 19 | Ethyl formate | 3 |
| 20 | Heptane | 3 |
| 21 | Isopropyl acetate | 3 |
| 22 | Methanol | 2 |
| 23 | Methylisobutyl ketone | 2 |
| 24 | N,N′-Dimethylacetamide | 2 |
| 25 | N,N′-Dimethylformamide | 2 |
| 26 | N-Methylpyrrolidone | 2 |
| 27 | Tetrahydrofuran | 2 |
| 28 | Toluene | 2 |
| 29 | Water | N/A |
| 30 | Acetone:Water (90:10% v/v) | 3 |
| 31 | Acetonitrile:Water (75:25% v/v) | 2 |
| 32 | Ethanol:Water (50:50% v/v) | 3 |
| 33 | 2-Propanol:Water (90:10% v/v) | 3 |
| 34 | Tetrahydrofuran:Water (98:2% v/v) | 2 |
| 35 | Dichloromethane:Methanol (50:50% v/v) | 2 |
| 36 | Dichloromethane:Methanol (75:25% v/v) | 2 |

Between each addition, the mixture was checked for dissolution and where no dissolution was apparent, the mixture was heated to ca. 40° C. and checked again. This procedure was continued until dissolution was observed or until 100 volumes of solvent had been added.

Where dissolution was not observed, solids were isolated centrifugally and analyzed by XRPD. The saturated solutions were analyzed by HPLC to obtain the solubility.

Where dissolution was noted, the clear solutions were left to evaporate at ambient conditions and the solids that were obtained were analyzed by XRPD.
High solubility of Compound A (above 100 mg/mL) was observed in 1,4-dioxane, DMSO, DMA, DMF and DCM:methanol (75:25% v/v).
Moderately high solubility of Compound A (between 100 and 50 mg/mL) was observed in DCM, NMP, THF, THF:water (98:2% v/v), and DCM:methanol (50:50% v/v).
Moderate solubility of Compound A (between 50 and 25 mg/mL) was observed for anisole.
The material was insoluble (<5 mg/mL) for the remaining 25 solvent systems tested.
Two new crystalline XRPD patterns were obtained during the solvent screen, named free base Pattern 1 and free base Pattern 2.

See Table 15 for a summary table of the solvent solubility screen.

TABLE 15
Summary of the solvent solubility screen

| | | Approximate | |
|---|---|---|---|
| | | Solubility | |
| No. | Solvent System | (mg/mL) | XRPD |
| 1 | 1-Butanol | 0.1881 | P1 |
| 2 | 1-Propanol | 0.2323 | P1 |
| 3 | 1,4-Dioxane | 100 | A |
| 4 | 2-Butanone (MEK) | 2.1332 | P1 |
| 5 | 2-Ethoxyethanol | 1.5534 | P1 |
| 6 | 2-Methyl THF | 2.8922 | A + p1 |
| 7 | 3-Methyl-1-butanol | 1.2200 | A |
| 8 | 2-Propanol | 0.2052 | A + p1 |
| 9 | Acetone | 2.7252 | P1 |
| 10 | Acetonitrile | 0.9948 | *P1 |
| 11 | Anisole | 25 | *P1 |
| 12 | tert-Butylmethyl ether | 0.1079 | A |
| 13 | Butyl acetate | 0.2087 | P1 |
| 14 | Dichloromethane | 50 | A |
| 15 | Dimethylsulfoxide | 100 | N/A |
| 16 | Ethanol | 0.2761 | P1 |
| 17 | Ethyl acetate | 0.4438 | P1 |
| 18 | Diethyl ether | 0.0020 | *P1 |
| 19 | Ethyl formate | 1.0127 | *P1 |
| 20 | Heptane | 0.0017 | A |
| 21 | Isopropyl acetate | 0.1742 | *P1 |
| 22 | Methanol | 0.4366 | *P2 |
| 23 | Methylisobutyl ketone | 0.5305 | *P1 |
| 24 | N,N′-Dimethylacetamide | 100 | N/A |
| 25 | N,N′-Dimethylformamide | 100 | N/A |
| 26 | N-Methylpyrrolidone | 50 | N/A |
| 27 | Tetrahydrofuran | 50 | *P1 |
| 28 | Toluene | 0.0298 | A |
| 29 | Water | 0.0005 | A |
| 30 | Acetone:Water (90:10% v/v) | 5.6779 | N/A |
| 31 | Acetonitrile:Water (75:25% v/v) | 1.0507 | A |
| 32 | Ethanol:Water (50:50% v/v) | 0.0192 | N/A |
| 33 | 2-Propanol:Water (90:10% v/v) | 0.1862 | *P2 |
| 34 | Tetrahydrofuran:Water (98:2% v/v) | 50 | N/A |
| 35 | Dichloromethane:Methanol (50:50% v/v) | 50 | A |
| 36 | Dichloromethane:Methanol (75:25% v/v) | 100 | A |

A: Amorphous,
P1: Pattern 1,
P2: Pattern 2,
*Poorly crystalline,
+ p1: some pattern 1 peaks

### Example 4. Salt Screen of Compound A

30 mg samples of Compound A were weighed out into 2 mL screw cap sample vials for 96 experiments. To these, 200 μL of the appropriate solvent system was added to dissolve the material or form a slurry. See Table 16 for the solvent systems selected for the primary salt screen.

TABLE 16
Selected solvent systems for the primary salt screen

| | Approximate |
|---|---|
| | solubility |
| Solvent system | (mg/mL) |
| 1,4-Dioxane | 100 |
| DCM | 50 |
| Dichloromethane:Methanol (50:50% | 50 |
| v/v) | |
| DMSO:Water (50:50% v/v) | Not determined |
| THF | 50 |
| Tetrahydrofuran:Water (98:2% v/v) | 50 |

1.05 eq. of solid counterions were weighed into six separate vials each and 1 molar stock solutions were prepared for liquid counterions in 5 mL of THF. See Table 17 for selected counterions and mass/volume needed for 1.05 eq. additions.
100 μL of the appropriate solvent was added to the solid counterions.

TABLE 17
Selected counterions for the primary salt screen

| | | | | | | | Neat addition |
|---|---|---|---|---|---|---|---|
| | | | | | | | 1.05 eq. (mg |
| | | | pKa | | | MW | or μL of 1 molar |
| No. | Counterion | Class | 1 | 2 | 3 | (g/mol) | stock solution) |
| 1 | Hydrochloric | 1 | −6.1 | | | 36.46 | 39 |
| | acid | | | | | | |
| 2 | Sulfuric acid | 1 | −3 | 1.92 | | 98.08 | 39 |
| 3 | p- | 2 | −1.34 | | | 190.22 | 7.65 |
| | Toluenesulfonic | | | | | | |
| | acid | | | | | | |
| | monohydrate | | | | | | |
| 4 | Methanesulfonic | 2 | −1.2 | | | 96.1 | 39 |
| | acid | | | | | | |
| 5 | Benzenesulfonic | 2 | 0.7 | | | 158.18 | 6.85 |
| | acid | | | | | | |
| 6 | Maleic acid | 1 | 1.92 | 6.23 | | 116.07 | 4.57 |
| 7 | Phosphoric acid | 1 | 1.96 | 7.12 | 12.32 | 98 | 39 |
| 8 | L-Glutamic acid | 1 | 2.19 | 4.25 | | 147.13 | 5.79 |
| 9 | Malonic acid | 2 | 2.83 | 5.7 | | 104.06 | 4.10 |
| 10 | L-Tartaric acid | 1 | 3.02 | 4.36 | | 150.09 | 5.88 |
| 11 | Fumaric acid | 1 | 3.03 | 4.38 | | 116.07 | 4.57 |
| 12 | Citric acid | 1 | 3.13 | 4.76 | 6.4 | 192.12 | 7.53 |
| 13 | L-Malic acid | 1 | 3.46 | 5.1 | | 134.09 | 5.39 |
| 14 | Benzoic acid | 2 | 4.19 | | | 122.12 | 4.78 |
| 15 | Succinic acid | 1 | 4.21 | 5.64 | | 118.09 | 4.65 |
| 16 | Acetic acid | 1 | 4.76 | | | 60.05 | 39 |

The solution/slurry of counterion was added to the solution/slurry of the free base material.

This was mixed using a vortex mixer and observations were noted, see Table 18.

TABLE 18
Observations made before temperature cycling

| | Hydrochloric | Sulfuric | p-Toluenesulfonic | Methanesulfonic | Benzenesulfonic | Maleic | Phosphoric | l- |
|---|---|---|---|---|---|---|---|---|
| | acid | acid | acid | acid | acid | acid | acid | Glutamic |
| 1,4-Dioxane | S | S WS | S WS | S WS | S WS | S | M | S |
| DCM | S | S WS | S | S WS | S | S | M | S |
| DCM:Methanol | S | S | S | S | N/A | | S | S |
| (50:50 % | | | | | | | | |
| v/v) | | | | | | | | |
| DMSO:Water | S | S | S | S | S | S | S | S |
| (50:50 % | | | | | | | | |
| v/v) | | | | | | | | |
| THF | S | S WS | S | S | S | S | M | S |
| THF:Water | S | S WS | S | S | S | S | S | S |
| (98:2 % | | | | | | | | |
| v/v) | | | | | | | | |

| | Malonic | l-Tartaric | Fumaric | Citric | l-Malic | Benzoic | Succinic | Acetic |
|---|---|---|---|---|---|---|---|---|
| | acid | acid | acid | acid | acid | acid | acid | acid |
| 1,4-Dioxane | S | S WS | S | S | S | S | S | S |
| DCM | | S | S | S | S | S | S | S |
| DCM:Methanol | | | | S | S | S | S | S |
| (50:50 % | | | | | | | | |
| v/v) | | | | | | | | |
| DMSO:Water | S | S | S | S | S | S | S | M |
| (50:50 % | | | | | | | | |
| v/v) | | | | | | | | |
| THF | S | S | | S | S | S | S | S |
| THF:Water | S | S | S | S | S | S | S | S |
| (98:2 % | | | | | | | | |
| v/v) | | | | | | | | |

S—slurry;
M—milky slurry;
WS—white solids

The samples were placed in an incubator shaker to temperature cycle between ambient and 40° C. over 4 hour cycles.
After ca. 72 hours of temperature cycling, observations were noted, see Table 19.

TABLE 19
Observations made after temperature cycling

| | Hydrochloric | Sulfuric | p-Toluenesulfonic | Methanesulfonic | Benzenesulfonic | Maleic | Phosphoric | l- |
|---|---|---|---|---|---|---|---|---|
| | acid | acid | acid | acid | acid | acid | acid | Glutamic |
| 1,4-Dioxane | S | S | G | G | G | S | S | S |
| DCM | S | S | | | | | S | |
| DCM:Methanol | S | | | | N/A | | S | |
| (50:50 % | | | | | | | | |
| v/v) | | | | | | | | |
| DMSO:Water | S | G | S | G | | G | S | S |
| (50:50 % | | | | | | | | |
| v/v) | | | | | | | | |
| THF | | S | S | S | | | S | |
| THF:Water | S | | | | | | S | |
| (98:2 % | | | | | | | | |
| v/v) | | | | | | | | |

| | Malonic | l-Tartaric | Fumaric | Citric | l-Malic | Benzoic | Succinic | Acetic |
|---|---|---|---|---|---|---|---|---|
| | acid | acid | acid | acid | acid | acid | acid | acid |
| 1,4-Dioxane | S | G | S | G | | S | S | S |
| DCM | | | | | | | | |
| DCM:Methanol | | | | | | | S | S |
| (50:50 % | | | | | | | | |
| v/v) | | | | | | | | |
| DMSO:Water | G | S | S | G | S | S | S | S |
| (50:50 % | | | | | | | | |
| v/v) | | | | | | | | |
| THF | | | | | | | | |
| THF:Water | | | | | | | | S |
| (98:2 % | | | | | | | | |
| v/v) | | | | | | | | |

S—slurry;
G—gel

Where slurries were obtained, the solids were isolated via centrifugation. Solids and gels were analyzed by XRPD.

Any samples that were clear solutions after temperature cycling were left to evaporate at ambient conditions. See Table 20 for observations made after evaporation. Solids obtained through evaporation were analyzed by XRPD.

TABLE 20
Observations made after evaporation

| | Hydrochloric | Sulfuric | p-Toluenesulfonic | Methanesulfonic | Benzenesalfonic | Maleic | Phosphoric | l- |
|---|---|---|---|---|---|---|---|---|
| | acid | acid | acid | acid | acid | acid | acid | Glutamic |
| 1,4- | N/A | N/A | N/A | N/A | N/A | N/A | N/A | N/A |
| Dioxane | | | | | | | | |
| DCM | N/A | N/A | S | S | S | S | N/A | S |
| DCM:Methanol | N/A | S | S | | N/A | G | N/A | S |
| (50:50 % | | | | | | | | |
| v/v) | | | | | | | | |
| DMSO:Water | N/A | N/A | N/A | N/A | Slurry | N/A | N/A | N/A |
| (50:50 % | | | | | | | | |
| v/v) | | | | | | | | |
| THF | S | N/A | N/A | N/A | N/A | G | N/A | S |
| THF:Water | N/A | G | S | S | S | G | N/A | S |
| (98:2 % | | | | | | | | |
| v/v) | | | | | | | | |

| | Malonic | l-Tartaric | Fumaric | Citric | l-Malic | Benzoic | Succinic | Acetic |
|---|---|---|---|---|---|---|---|---|
| | acid | acid | acid | acid | acid | acid | acid | acid |
| 1,4- | N/A | N/A | N/A | N/A | G | N/A | N/A | N/A |
| Dioxane | | | | | | | | |
| DCM | S | S | S | S | S | G | G | S |
| DCM:Methanol | G | S | S | G | S | S | N/A | N/A |
| (50:50 % | | | | | | | | |
| v/v) | | | | | | | | |
| DMSO:Water | N/A | N/A | N/A | N/A | N/A | N/A | N/A | N/A |
| (50:50 % | | | | | | | | |
| v/v) | | | | | | | | |
| THF | G | S | G | G | G | G | G | S |
| THF:Water | G | S | S | G | G | G | G | N/A |
| (98:2 % | | | | | | | | |
| v/v) | | | | | | | | |

S—solids;
G—gel

The samples obtained from temperature cycling were dried under vacuum at 40° C. for ca. 20 hours while the samples obtained from evaporation were dried under vacuum at 40° C. for ca. 4 hours. The samples were then analyzed by XRPD.
The XRPD plate was then stored at 40° C./75% RH for ca. 20 hours and reanalyzed by XRPD.
After initial XRPD analysis of the solids obtained during the primary salt screen, potential salt forms were obtained for hydrochloric acid, p-toluenesulfonic acid, benzenesulfonic acid, maleic acid, phosphoric acid and fumaric acid.
   The potential fumarate salt fell to amorphous after drying.
   The potential maleate salt fell to amorphous after elevated humidity.
   CAD analysis of the potential chloride salts found only trace amounts of chloride.
   The tosylate, besylate, and phosphate all appear to be stable hydrates.
Additionally, two new possibly free base patterns were obtained from 1,4-dioxane samples.
   Pattern 3 from hydrochloric acid, l-glutamic acid, succinic acid, and acetic acid.

Pattern 4 from malonic acid and benzoic acid.

See Tables 21-23 for a summary of the XRPD results of the primary salt screen.

TABLE 21
Summary of XRPD results post temperature cycling and evaporation

| | Hydrochloric | Sulfuric | p-Toluenesulfonic | Methanesulfonic | Benzenesulfonic | Maleic | Phosphoric | l- |
|---|---|---|---|---|---|---|---|---|
| | acid | acid | acid | acid | acid | acid | acid | Glutamic |
| 1,4-Dioxane | t * 3 | A t | A t | A t | A t | S t * | A t | C e * 3 |
| DCM | t S | A t | A e | A e | A e | C e | S t * | C e |
| DCM:Methanol | t S | A e | A e | N/A | N/A | A e | S t * | e 2 |
| (50:50 % | | | | | | | | |
| v/v) | | | | | | | | |
| DMSO:Water | t 2 | A t | A t | A t | A e | A t | t * 2 | A t |
| (50:50 % | | | | | | | | |
| v/v) | | | | | | | | |
| THF | e 2 | A t | S t | t 2 | A t | A e | S t | C e 2 |
| THF:Water | t 2 | N/A | A e | A e | S e | A e | S t | e * 2 |
| (98:2 % | | | | | | | | |
| v/v) | | | | | | | | |

| | Malonic | l-Tartaric | Fumaric | Citric | l-Malic | Benzoic | Succinic | Acetic |
|---|---|---|---|---|---|---|---|---|
| | acid | acid | acid | acid | acid | acid | acid | acid |
| 1,4-Dioxane | t * 4 | A t | S t * | A t | e * 3 | t 4 | t * 3 | t * 3 |
| DCM | A e | A e | A e | A e | A e | A e | C e | e * 2 |
| DCM:Methanol | A e | A e | A e | A e | e * 2 | e 2 | t 2 | t 2 |
| (50:50 % | | | | | | | | |
| v/v) | | | | | | | | |
| DMSO:Water | A t | A t | t * 2 | A t | A t | t * 2 | A t | t * 2 |
| (50:50 % | | | | | | | | |
| v/v) | | | | | | | | |
| THF | A e | A e | A e | A e | A e | e * 2 | e * 2 | e * 2 |
| THF:Water | A e | A e | e * 2 | A e | e * 2 | e * 2 | e * 2 | t * 1 |
| (98:2 % | | | | | | | | |
| v/v) | | | | | | | | |

1—Free base Pattern 1,
2—Free base Pattern 2,
3—Free base Pattern 3,
4—Free base Pattern 4,
S—Potential Salt Pattern 1,
A—Amorphous,
C—Counterion present,
*—Poorly crystalline,
t—Results obtained from temperature cycling,
e—Results obtained from evaporation

TABLE 22
Summary of XRPD results post drying

| | Hydrochloric | Sulfuric | p-Toluenesulfonic | Methanesulfonic | Benzenesulfonic | Maleic | Phosphoric | l- |
|---|---|---|---|---|---|---|---|---|
| | acid | acid | acid | acid | acid | acid | acid | Glutamic |
| 1,4-Dioxane | t * 2 | A t | A t | A t | A t | S1 t * | A t | C e * 3 |
| DCM | S2 t d | A t | A e | A e | A e | A e d | S1 t * | C e |
| DCM:Methanol | S2 t d | A e | A e | N/A | N/A | A e | S1 t * | e 2 |
| (50:50 % | | | | | | | | |
| v/v) | | | | | | | | |
| DMSO:Water | t 2 | A t | A t | A t | S1 e d | A t | t 1 d | t 1 d |
| (50:50 % | | | | | | | | |
| v/v) | | | | | | | | |
| THF | e 2 | A t | S1 t | t 2 | A t | A e | S1 t * | C e 2 |
| THF:Water | t 2 | N/A | A e | A e | S1 e | A e | S1 t * | e * 2 |
| (98:2 % | | | | | | | | |
| v/v) | | | | | | | | |

| | Malonic | l-Tartaric | Fumaric | Citric | l-Malic | Benzoic | Succinic | Acetic |
|---|---|---|---|---|---|---|---|---|
| | acid | acid | acid | acid | acid | acid | acid | acid |
| 1,4-Dioxane | t * 4 | A t | A t d | A t | e * 3 | t 4 | t * 3 | t * 3 |
| DCM | A e | A e | A e | A e | A e | A e | A e d | e * 2 |
| DCM:Methanol | A e | A e | A e | A e | e 2 | e 2 | t 2 | t 2 |
| (50:50 % | | | | | | | | |
| v/v) | | | | | | | | |
| DMSO:Water | t 1 d | t 1 d | t 1 d | A t | t 1 d | t 1 d | t 1 d | t * 2 |
| (50:50 % | | | | | | | | |
| v/v) | | | | | | | | |
| THF | A e | A e | A e | A e | A e | e * 2 | e * 2 | e * 2 |
| THF:Water | A e | A e | e * 2 | A e | e * 2 | e * 2 | e * 2 | t * 1 |
| (98:2 % | | | | | | | | |
| v/v) | | | | | | | | |

1—Free base Pattern 1,
2—Free base Pattern 2,
3—Free base Pattern 3,
P4—Free base Pattern 4,
S1—Potential Salt Pattern 1,
S2—Potential Salt Pattern 2,
A—Amorphous,
C—Counterion present,
*—Poorly crystalline,
t—Results obtained from temperature cycling,
e—Results obtained from evaporation,
d—XRPD changed upon drying

TABLE 23
Summary of XRPD results post 40° C./75% RH

| | Hydrochloric | Sulfuric | p-Toluenesulfonic | Methanesulfonic | Benzenesulfonic | Maleic | Phosphoric | l- |
|---|---|---|---|---|---|---|---|---|
| | acid | acid | acid | acid | acid | acid | acid | Glutamic |
| 1,4-Dioxane | t * 2 | A t | A t | A t | A t | A t h | A t | C e * 3 |
| DCM | S2 t | A t | A e | A e | A e | A e | A t h | C e |
| DCM:Methanol | S2 t | A e | A e | N/A | N/A | A e | S1 t * | e 2 |
| (50:50 % | | | | | | | | |
| v/v) | | | | | | | | |
| DMSO:Water | A t h | A t | A t | A t | A e h | A t | t 1 | t 1 |
| (50:50 % | | | | | | | | |
| v/v) | | | | | | | | |
| THF | e 2 | A t | S1 t | t 2 | A t | A e | S1 t * | C e 2 |
| THF:Water | t 2 | N/A | A e | A e | S1 e | A e | S1 t * | e * 2 |
| (98:2 % | | | | | | | | |
| v/v) | | | | | | | | |

| | Malonic | l-Tartaric | Fumaric | Citric | l-Malic | Benzoic | Succinic | Acetic |
|---|---|---|---|---|---|---|---|---|
| | acid | acid | acid | acid | acid | acid | acid | acid |
| 1,4-Dioxane | t * 4 | A t | A t | A t | e * 3 | t 4 | t * 3 | t * 3 |
| DCM | A e | A e | A e | A e | A e | A e | C e h | e * 2 |
| DCM:Methanol | A e | A e | A e | A e | e 2 | e 2 | t 2 | t 2 |
| (50:50 % | | | | | | | | |
| v/v) | | | | | | | | |
| DMSO:Water | t 1 | t 1 | t 1 | A t | t 1 | t 1 | t 1 | A t h |
| (50:50 % | | | | | | | | |
| v/v) | | | | | | | | |
| THF | A e | A e | A e | A e | A e | e * 2 | e * 2 | e * 2 |
| THF:Water | A e | A e | e * 2 | A e | e * 2 | e * 2 | e * 2 | t * 1 |
| (98:2 % | | | | | | | | |
| v/v) | | | | | | | | |

1—Free base Pattern 1,
2—Free base Pattern 2,
3—Free base Pattern 3,
P4—Free base Pattern 4,
S1—Potential Salt Pattern 1,
S2—Potential Salt Pattern 2,
A—Amorphous,
C—Counterion present,
*—Poorly crystalline,
t—Results obtained from temperature cycling,
e—Results obtained from evaporation,
h—XRPD changed after elevated humidity

### Example 5. Characterization of Novel Forms

Potential chloride salt Pattern 2, tosylate Pattern 1, besylate Pattern 1, and phosphate Pattern 1 were characterized by the following techniques:
PLM
FT-IR
TG/DSC
CAD (where applicable)
¹H NMR (where applicable)
³¹P NMR (where applicable)

### Potential Chloride Salt Pattern 2

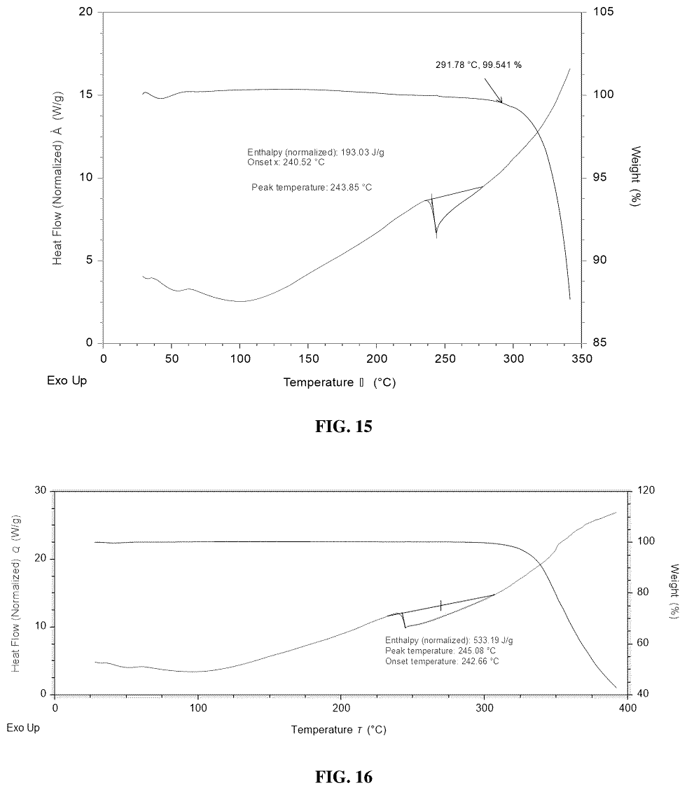

Potential chloride salt Pattern 2 was observed after drying potential chloride salt Pattern 1 which was obtained from DCM and DCM:methanol. Drying was carried out in a 40° C. oven for ca. 20 hours.
PLM showed birefringent rod-like particles.
FT-IR analysis showed slight peak shifting compared to the amorphous free base. A C—C triple bond peak was evident at 2218 cm⁻¹. Amide peaks and possible presence of water was noted at higher wavenumbers. See FIG. 14.
By TG/DSC on the sample from DCM, no notable mass losses were observed in the TG trace prior to decomposition above 292° C. indicating that the material was anhydrous. In the DSC trace, a sharp endothermic event was noted with an onset at 241° C., likely due to melting and similar to the melt observed for free base Pattern 1. See FIG. 15.
The TG/DSC analysis was repeated on the sample from DCM:methanol. In the TG trace, no notable mass losses were observed prior to decomposition above 310° C. In the DSC trace, a broad melting endothermic event was observed with an onset at 243° C. which matches well with the previously obtained thermogram. See FIG. 16.
CAD analysis of the two potential chloride salt samples (from DCM and from DCM:methanol (50:50% v/v)) determined there were only trace amounts of chloride present.
   The potential chloride salt Pattern 2 was not a salt.

### Tosylate Pattern 1

By PLM, no clear morphology was observed and agglomeration was noted. The material appeared birefringent under polarized light.
FT-IR analysis determined there were some slight differences in the spectra, likely due to the presence of p-toluenesulfonic acid. A C—C triple bond peak was evident at 2220 cm⁻¹. No clear presence of water of a hydrate was observed. See FIG. 17.
TG/DSC analysis found a gradual 1.8 wt. % (0.25 eq. THF or 1 eq. water) mass loss in the TG trace from the onset of heating up to the potential melting event. Decomposition was observed above 278° C. An endothermic event likely due to melting was noted in the DSC trace with onset 195° C. See FIG. 18.
The ¹H NMR showed a 1:1 ratio of p-toluenesulfonic acid to Compound A. No significant amount of THF was observed in the ¹H NMR, indicating that the mass loss from the TG/DSC was likely due to water. No peak shifting was observed compared to the free base. See FIG. 19.
   Tosylate Pattern 1 was a hydrated salt with a 1:1 stoichiometry of p-toluenesulfonic acid to Compound A.

### Besylate Pattern 1

By PLM, large pink plate-like particles were noted. The material appeared weakly birefringent under polarised light.
FT-IR analysis determined there were some slight differences in the spectra, likely due to the presence of benzenesulfonic acid. C—C triple bond peak evident at 2222 cm⁻¹. See FIG. 20.
By TG/DSC, two mass losses were observed in the TG trace from the onset of heating to approximately 277° C.: 4.2 wt. % mass loss (0.6 eq. of THF or 2.4 eq. of water) and a 3.7 wt. % mass loss (0.5 eq. of THF or 2.1 eq. of water). Decomposition was observed above 277° C. In the DSC trace, there was no clear evidence of melting. A broad endothermic event was observed during the first mass loss, possibly related to de-solvation. See FIG. 21.
The ¹H NMR showed a 1:1 ratio of benzenesulfonic acid to Compound A. 0.85 wt. % (0.11 eq.) of THF was observed in the ¹H NMR. Minor peak shifting was observed when compared to Compound A free base. See FIG. 22.
   Besylate Pattern 1 was a hydrated salt with a 1:1 stoichiometry of benzenesulfonic acid to Compound A.

### Phosphate Pattern 1

PLM analysis observed no clear morphology. Agglomeration was noted and the material appeared weakly birefringent under polarized light.
FT-IR analysis determined that the spectra were comparable, with some slight peak shifting observed likely due to the presence of phosphoric acid. The C—C triple bond peak was evident at 2221 cm⁻¹. No clear presence of water of a hydrate was found in the spectrum. See FIG. 23.
TG/DSC analysis found a gradual mass loss of 5.8 wt. % (3 eq. water or 0.8 eq. THF) in the TG trace noted from the outset to approximately 170° C. Decomposition was noted above 249° C. In the DSC trace, a broad endothermic event occurred after the mass loss between 165° C. and 245° C. See FIG. 24.
The ¹H NMR was consistent with the structure. No significant amount of THF was observed, indicating that the mass loss from the TG/DSC was likely due to water. Minor peak shifting was observed when compared to Compound A free base and a broad water peak was present, both are indicative of salt formation. A peak was observed in the ³¹P NMR, confirming the presence of phosphate. See FIGS. 25 and 26. CAD analysis found 22.9 wt. % phosphate which corresponds to roughly 2 eq. of phosphate (1 eq.=10.5 wt. % and 2 eq.=18.9 wt. %)
   Phosphate Pattern 1 was a hydrated salt with a 2:1 stoichiometry of phosphoric acid to Compound A free base.

TABLE 24
Summary of the characterization carried out on the novel salt forms

| | Solvent | 40° C./75% | | | | |
|---|---|---|---|---|---|---|
| Salt | System | RH | PLM | TG/DSC | ¹H NMR | Comment |
| Tosylate | THF | Pattern 1 | No clear | Loss of 0.25 | 1:1 ratio of | Unclear |
| Pattern 1 | | retained | morphology; | equiv. of THF | free base to | if |
| | | | appeared | or 1 equiv. of | p- | hydrated |
| | | | birefringent | water when | toluenesulfonic | or |
| | | | | heated to melt | acid, no | anhydrous |
| | | | | onset (195° C.). | significant | |
| | | | | Decomposition | amount of | |
| | | | | onset ~278° C. | THF which | |
| | | | | | suggests the | |
| | | | | | mass loss in | |
| | | | | | the TG/DSC | |
| | | | | | was due to | |
| | | | | | water. No | |
| | | | | | peak shifting, | |
| | | | | | no broad | |
| | | | | | water peak. | |
| Besylate | THF/water | Pattern 1 | Large pink | Loss of 0.6 | 1:1 ratio of | Possible |
| Pattern 1 | (98:2 % | retained | plate-like | equiv. of THF | free base to | THF |
| | v/v) | | particles; | (or 2.4 equiv. of | benzenesulfonic | solvate |
| | | | appeared | water) when | acid. 0.85 | (or a |
| | | | weakly | heated to 140° | wt. % (0.11 | hydrate). |
| | | | birefringent | C. Steady loss | eq. THF). No | Amorphous |
| | | | | in mass up to ~277° | peak shifting, | content |
| | | | | C. where | broad water | observed. |
| | | | | decomposition | peak. | The |
| | | | | was evident. No | | material |
| | | | | clear melting | | is red. |
| | | | | event noted. | | |
| Phosphate | THF and | Pattern 1 | No clear | Steady loss of 3 | No | Possible |
| Pattern 1 | THF/water | retained but | morphology; | equiv. of water | significant | hydrate. |
| | (98:2 % | crystallinity | appeared | or 0.8 equiv. of | amount of | Some |
| | v/v) | slightly | birefringent | THF when | THF which | amorphous |
| | | reduced | | heated to ~170° | suggests the | content |
| | | | | C. | mass loss in | noted. |
| | | | | Decomposition | the TG/DSC | No clear |
| | | | | evident above ~249° | was due to | melt |
| | | | | C. Broad | water. No | onset |
| | | | | endothermic | peak shifting, | observed. |
| | | | | event observed | broad water | |
| | | | | between 165 | peak. | |
| | | | | and 245° C. | Phosphate | |
| | | | | following mass | peak present | |
| | | | | loss and prior to | in the ³¹P | |
| | | | | decomposition | NMR. CAD | |
| | | | | onset. | analysis | |
| | | | | | found 2 eq. | |
| | | | | | of phosphate. | |

### Aqueous Solubility

The aqueous solubility of the promising salts was determined in unbuffered water.
To 2.2 mg of tosylate salt, and 5 mg each of the besylate salt and phosphate salt, 0.5 mL of unbuffered water was added to obtain slurries. See Table 25.
Each sample was capped and sealed in parafilm and placed in an incubator shaker at 25° C. for ca. 24 hours.
After 24 hours, observations were noted (see Table 25) and the slurries were filtered centrifugally. The saturated solutions were submitted for HPLC analysis and were injected neat due to the low solubility.

TABLE 25
Aqueous solubility observations

| | Volume of | | |
|---|---|---|---|
| | water | Initial | Observations |
| Salt | (mL) | observations | post shaking |
| Tosylate Pattern 1 | 0.5 | Purple slurry | Purple slurry |
| Besylate Pattern 1 | 0.5 | Red slurry | Red slurry |
| Phosphate Pattern 1 | 0.5 | Purple slurry | Purple slurry |

Unfortunately, the solubility of all three samples were below the LOQ and thus could not be quantified.

### Example 6. Additional Chloride Salt Formation Experiments

Three additional chloride salt formation experiments were conducted to further investigate the possibility of stable chloride salt formation which appeared elusive, despite the large pKa difference between Compound A and hydrochloric acid. (Table 26)

TABLE 26
Summary of additional chloride formation experiments

| | | Free base | | Volume of | |
|---|---|---|---|---|---|
| | | mass per | | solvent | Equivalents |
| No. | Acid | vial (mg) | Solvent system | (μL) | of HCl |
| 1 | Hydrochloric | 30 | DCM:Methanol | 400 | 1.05 eq. 1M |
| | acid | | (50:50% v/v) | | HCl in THF |
| 2 | Hydrochloric | 30 | DCM | 400 | 2.1 eq. 1M |
| | acid | | | | HCl in THF |
| 3 | Hydrochloric | 200 | DCM:Methanol | 2000 | 2 eq. 1.25M |
| | acid | | (90:10% v/v) | | HCl in MeOH |

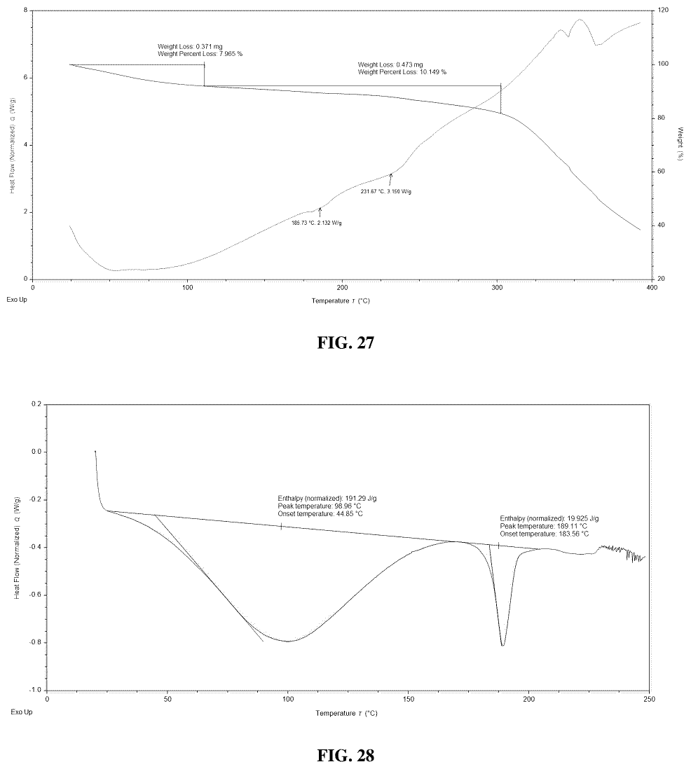

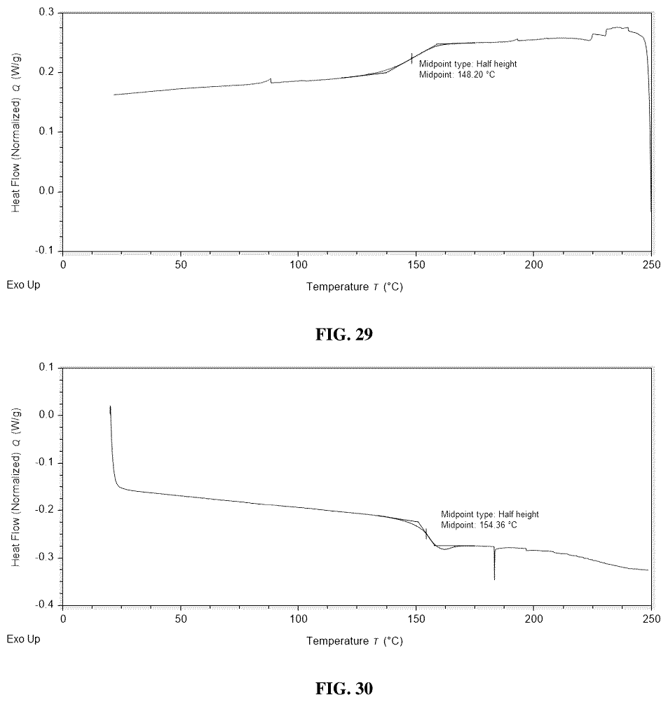

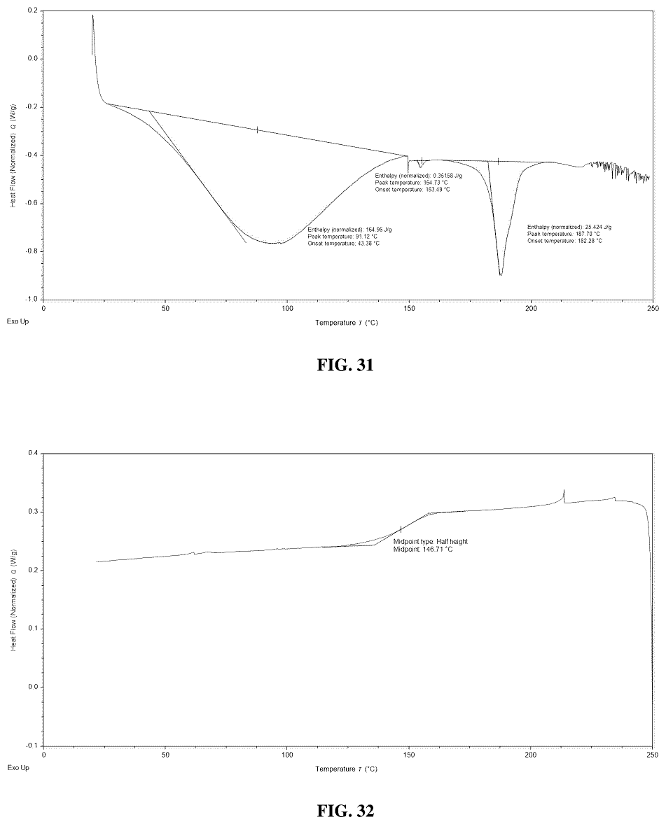

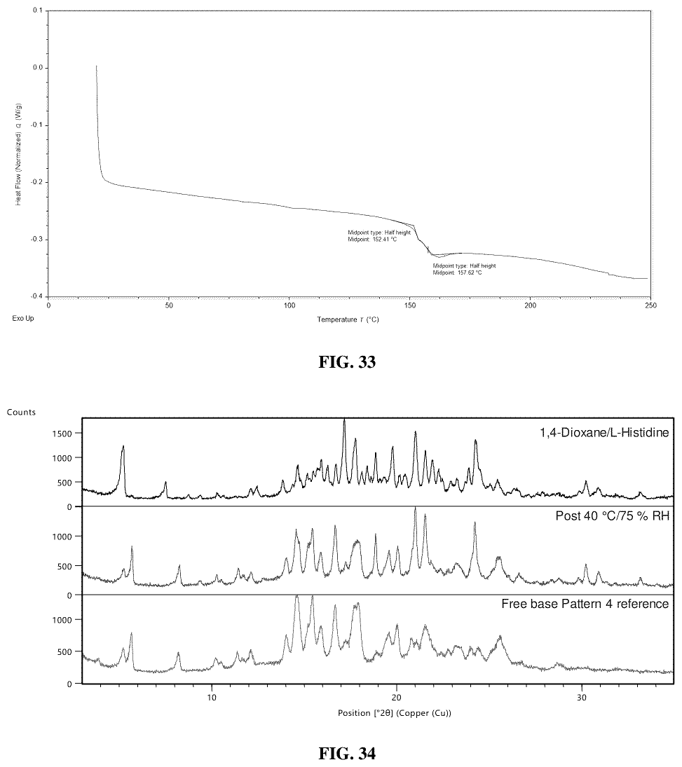

Experiment 1:30 mg of free base was dissolved in 400 μL of DCM:methanol (50:50% v/v). A clear purple solution was noted. 1.05 eq. of 1 M HCl in THF was added to the clear solution. No change was observed. The solution was capped and sealed in parafilm and then placed in an incubator shaker to temperature cycle between ambient and 50° C. over 4 hours cycles for 72 hours. An orange solution was obtained, the sample was uncapped and was left to evaporate.
Experiment 2:30 mg of free base was dissolved in 400 μL of DCM. A clear purple solution was noted. 2.1 eq. of 1 M HCl in THF was added. No change was observed. The solution was capped and sealed in parafilm and then placed in an incubator shaker to temperature cycle between ambient and 40° C. over 4 hours cycles for 72 hours. A yellow solution was obtained, the sample was uncapped and was left to evaporate.
Experiment 3 followed the method:
   200 mg of free base was dissolved in 2 mL of 9:1 DCM:methanol. A clear purple solution was obtained. Another 1 mL of DCM was added.
   0.8 mL of 1.25 M HCl in methanol was added.
   The solution was stirred on a stirrer hotplate at room temperature for 1 hour.
   The solvent was removed using a rotary evaporator over a 40° C. water bath. A brown gel was obtained.
   5 mL of ethanol was added, an off-white slurry was obtained.
   The slurry was stirred at room temperature for ca. 2.5 hours.
   The slurry was filtered by Buchner filtration and the material left to dry on the filter bed for ca. 5 minutes.
   The off-white solids were then placed in a scintillation vial, covered in tissue paper and placed in a 40° C. oven to dry for ca. 96 hours.
The material obtained from these experiments were analyzed by XRPD, where amorphous material was obtained, these were analyzed by CAD to determine chloride content. The solids obtained from Experiment 1 yielded an amorphous mono-chloride salt. This sample was further analyzed by TG/DSC and DSC.
Experiment 1, performed at elevated temperature, yielded free base Pattern 2. The other two samples gave amorphous material.
CAD analysis found 0.8 wt. % chloride from experiment 2.
   Trace amount outside of the calibration range
CAD analysis found 7.7 wt. % chloride from experiment 3 which corresponds to roughly 2 eq. of chloride. (1 eq.=4.2 wt. %, 2 eq.=8.0 wt. %)
TG/DSC of the amorphous chloride salt from experiment 3 found a mass loss of 7.97 wt. % (4.23 eq. water, 0.90 eq. DCM, 2.38 eq. methanol, 1.66 eq. ethanol, or 2.09 eq. HCl) from the onset of the experiment until ca. 110° C. A gradual mass loss of 10.15 wt. % (5.52 eq. water, 1.17 eq. DCM, 3.11 eq. methanol, 2.16 eq. ethanol, or 2.73 eq. HCl) was observed from 110° C. to decomposition at ca. 300° C. likely due to degradation of the sample. This data suggests that the chloride anions were lost at the onset of the experiment. There were two broad endothermic events observed in the DSC trace with peaks at 186° C. and 232° C. (FIGS. 27-33).
The first heating cycle in the DSC obtained two endothermic events. A broad endothermic event with an onset at 45° C. and peak at 99° C. corresponding to the mass loss observed in the TG/DSC. The second endothermic event was sharp and likely corresponds to the melt with an onset at 184° C. and peak at 189° C.
   The melt was lower than any free base melt previously obtained, so the DSC was repeated to confirm the temperature. The repeat DSC gave the same results.
A glass transition with midpoint at 148° C. was observed in the cooling cycle
A glass transition with midpoint at 154° C. was observed in the second heating cycle.

### Example 7. Co-Crystal Screen of Compound A

### Dry Milling

Ca. 30 mg of Compound A was weighed into 48× 2 mL bead mill vials.
1.05 eq. of each co-former was added to six bead mill vials.
Two stainless steel bead mill balls were added to each vial and the samples were dry milled using the following procedure:
   6500 RPM
   40×60 s cycles (total milling time: 40 minutes)
   10 s breaks
A subsample from each co-former was analyzed by XRPD post dry milling.

### Temperature Cycling

Each sample was dissolved in the appropriate solvent.
The solutions were temperature cycled in an incubator shaker between ambient and 40° C. over 4 hour cycles for ca. 72 hours.
After 3 days, slurries were filtered centrifugally and all solids that were obtained were analyzed by XRPD.
After XRPD analysis, the XRPD plate was placed in a 40° C./75% RH for ca. 30 hours and then the samples were re-analyzed by XRPD.
Samples yielding a novel XRPD pattern were analyzed by TG/DSC and PLM.

### Storage at 40° C./75% RH

The XRPD plate containing the co-crystal screen samples was stored in a humidity chamber at 40° C./75% RH for ca. 24 hours and then the samples were re-analyzed by XRPD.

### Results

The co-crystal screen yielded free base Patterns 1, 2, 4, and a novel pattern.
The novel pattern was obtained from 5 different co-formers in 1,4-dioxane, indicating this was likely a novel free base pattern. It was named potential free base Pattern 9.
   See Table 27 for a summary of the XRPD results after temperature cycling.
Pattern 9 converted to free base Pattern 4 after storage at 40° C./75% RH.
   See Table 28 for a summary of the XRPD results post storage at 40° C./75% RH.

TABLE 27
Summary of the XRPD results obtained during the primary co-crystal screen post temperature cycling

| | Nicotinamide | Methylparaben | Urea | L-Tyrosine | L-Phenylalanine | L-Histidine | L-Proline | L-Tryptophan |
|---|---|---|---|---|---|---|---|---|
| Dry mill | A | A | A | A | A | X | X | A |
| 1,4-Dioxane | 4 | 4 * | 9 | 9 | 9 | 9 | 9 | 9 * |
| DCM | G | G | G | G | G | G | G | G |
| DCM:Methanol | 2 | 1 | 2 | 2 | 2 + | 2 | 2 | 2 |
| (50:50 % | | | | | | | | |
| v/v) | | | | | | | | |
| DMSO:Water | 2 | A | A | A | A | A | A | A |
| (50:50 % | | | | | | | | |
| v/v) | | | | | | | | |
| THF | 1 | 1 | 1 | 1 + | 1 + | 1 | 1 | 1 + |
| THF:Water | G | G | G | 2 * | G | 2 * | 1+* | 2+* |
| (98:2 % | | | | | | | | |
| v/v) | | | | | | | | |

A—Amorphous;
X—Predominantly amorphous with some potential free base pattern 9 peaks;
G—Glassy material on the bottom of the vial;
1—Free base Pattern 1;
2—Free base Pattern 2;
4—Free base Pattern 4;
9—Potential new free base Pattern 9;
+—Additional peaks observed (likely due to presence of co-former);
*—Poorly crystalline

TABLE 28
Summary of the XRPD results obtained during the primary co-crystal screen post storage at 40° C./75% RH

| | Nicotinamide | Methylparaben | Urea | L-Tyrosine | L-Phenylalanine | L-Histidine | L-Proline | L-Tryptophan |
|---|---|---|---|---|---|---|---|---|
| 1,4-Dioxane | 4 | 4 * | 4 | 4 | 4 | 4 | 4 | 4 * |
| DCM | G | G | G | G | G | G | G | G |
| DCM:Methanol | 2 | 1 | 2 | 2 | 2 + | 2 | 2 | 2 + |
| (50:50 % | | | | | | | | |
| v/v) | | | | | | | | |
| DMSO:Water | 2 | 2 * | 2 * | 2 * | 2+* | 2 * | 2 * | 2 * |
| (50:50 % | | | | | | | | |
| v/v) | | | | | | | | |
| THF | 1 | 1 | 1 | 1 + | 1 + | 1 | 1 | 1 + |
| THF:Water | G | G | G | 2 * | G | 2 * | 1+* | 2+* |
| (98:2 % | | | | | | | | |
| v/v) | | | | | | | | |

G—Glassy material on the bottom of the vial;
1—Free base Pattern 1;
2—Free base Pattern 2;
4—Free base Pattern 4;
+—Additional peaks observed (likely due to presence of co-former);
*—Poorly crystalline

### Characterization of Free Base Pattern 9

Novel free base Pattern 9 converts to Pattern 4 post storage at 40° C./75% RH, see FIG. 34.
Very small (˜5 μm) weakly birefringent particles were observed by PLM. Agglomeration was noted.
TG/DSC determined there were two mass losses in the TG trace. The first was an 8.2 wt. % (4 eq. of water, 0.82 eq. 1,4-dioxane, or 0.5 eq. L-histidine) mass loss and occurred between 55° C. and 175° C. The second was a 3.8 wt. % (1.75 eq. water, 0.36 eq. 1,4-dioxane, or 0.2 eq. L-histidine) mass loss lost between 255° C. and 290° C. Decomposition onset above 289° C. There were several events in the DSC trace. An exothermic event with onset: 146° C., peak: 164° C. A second exothermic event with onset: 187° C., peak: 195° C. An endothermic event with onset: 238° C., peak: 243° C. A second endothermic event with onset: 264° C., peak: 271° C. See FIG. 35 for the TG/DSC.

### Example 8. Crystallization Screen of Compound A

34×40 mg of Compound A was weighed into vials before the selected solvents were added to prepare slurries, where possible. See Table 29 for a list of solvent systems used and the volume of solvent used.
Stirrer bars were added to the vials, before the vials were sealed and temperature cycled with stirring between 5° C. and 40° C. The heating rate was 0.1° C./min and the samples were held at 5° C. and 40° C. for 2 hours.
After temperature cycling for 4 days, observations were made (see Table 29). Slurries were centrifuge filtered and the solids were analyzed by XRPD
   The solids were dried at 40° C. for approximately 18 h before being re-analyzed by XRPD.
Saturated solutions were then split into 3 vials for the following experiments
   Evaporation at ambient temperature
   Cooling (5° C.).
      Where no solids were obtained after 72 hours, the samples were moved to the freezer at −20° C. for further cooling.
      Where no solids were obtained from further cooling, the samples were allowed to evaporate at ambient temperature.
Anti-solvent addition at ambient temperature.
   Where no solids were obtained after 72 hours the samples were moved to the freezer at −20° C. to encourage crystallization.
   Where no solids were obtained from cooling, the samples were allowed to evaporate at ambient temperature.

Free base forms were characterized by PLM, TG/DSC, and ¹H NMR.

TABLE 29
List of solvent systems used in the primary crystallization screen
with the volume used and observations post temperature cycling

| | | Volume of | |
|---|---|---|---|
| | | solvent | Observations post |
| No. | Solvent System | (mL) | temperature cycling |
| 1 | 1-Butanol | 20 | Off-white slurry |
| 2 | 1-Propanol | 20 | Off-white slurry |
| 3 | 1,4-Dioxane | 0.3 | Off-white solid, orange |
| | | | solution |
| 4 | 2-Butanone (MEK) | 20 | Colorless solution |
| 5 | 2-Ethoxyethanol | 20 | Off-white, thin slurry |
| 6 | 2-Methyl THF | 20 | Colorless solution |
| 7 | 3-Methyl-1-butanol | 20 | Off-white slurry |
| 8 | 2-Propanol | 20 | Off-white slurry |
| 9 | Acetone | 20 | Colorless solution |
| 10 | Acetonitrile | 20 | Off-white slurry |
| 11 | Anisole | 0.3 | Off-white solid, grey solution |
| 12 | tert-Butylmethyl ether | 20 | Off-white slurry |
| 13 | Butyl acetate | 20 | Off-white slurry |
| 14 | Dichloromethane | 0.3 | Off-white solid, grey solution |
| 15 | Ethanol | 20 | Off-white slurry |
| 16 | Ethyl acetate | 20 | Off-white slurry |
| 17 | Diethyl ether | 20 | Off-white slurry |
| 18 | Ethyl formate | 20 | Off-white slurry |
| 19 | Isopropyl acetate | 20 | Off-white slurry |
| 20 | Methanol | 20 | Off-white slurry |
| 21 | Methylisobutyl ketone | 20 | Off-white slurry |
| 22 | N,N′-Dimethylacetamide | 0.3 | Pale brown solution |
| 23 | N,N′-Dimethylformamide | 0.3 | Pale brown solution |
| 24 | N-Methylpyrrolidone | 0.3 | Orange solution |
| 25 | Tetrahydrofuran | 0.3 | Off-white solid, grey solution |
| 26 | Toluene | 20 | Off-white, thin slurry |
| 27 | Water | 20 | Off-white slurry |
| 28 | Acetone:Water (90:10% v/v) | 20 | Colorless solution |
| 29 | Acetonitrile:Water (75:25% v/v) | 20 | Off-white slurry |
| 30 | Ethanol:Water (50:50% v/v) | 20 | Off-white slurry |
| 31 | 2-Propanol:Water (90:10% v/v) | 20 | Off-white slurry |
| 32 | Tetrahydrofuran:Water (98:2% v/v) | 0.3 | Yellow solution |
| 33 | Dichloromethane:Methanol (50:50% v/v | 0.3 | Off-white solid, grey solution |
| 34 | Dichloromethane:Methanol (75:25% v/v) | 0.3 | Grey/purple solution |

Six free base Patterns were elucidated in the primary crystallization screen.
   Patterns 1 and 2 were previously observed.
   Patterns 5 through 8 were newly obtained.
   Patterns 3 and 4 which were obtained in the salt screen and Pattern 9 which was obtained in the co-crystal screen were not observed in the crystallization screen.
Free base Pattern 1 was most commonly recovered from the experiments.
Patterns 5 and 7 converted to Pattern 8 upon drying at 40° C.

See Table 30 for a summary of the XRPD Patterns obtained during the primary crystallization screen.

TABLE 30
Summary of the XRPD Patterns obtained during the primary crystallization screen

| | | Post- | | | |
|---|---|---|---|---|---|
| | Post- | Thermal | | | Anti- |
| | Thermal | Cycling | | | Solvent |
| Solvent | Cycling | (dried) | Evaporation | Cooling | Addition |
| 1-Butanol | 1 * | 1 * | | | |
| 1-Propanol | 1 * | 1 * | A | | |
| 1,4-Dioxane | A | A * | A x | A e | A |
| 2-Butanone (MEK) | | | 1 | 1 * e | 1 * e |
| 2-Ethoxyethanol | 1 * | 1 * | 1 * | | |
| 2-Methyl THF | | | 1 * | 1 e | 1 * |
| 3-Methyl-1-butanol | 1 * | 1 * | | | |
| 2-Propanol | 1 * | 1 * | | | |
| Acetone | | | 1 * | 2 * e | 1 e |
| Acetonitrile | 1 | 1 | A + | | A x |
| Anisole | 5 | 8 | | | |
| tert-Butylmethyl ether | A; Some | A; Some | | | |
| | peaks of 1 | peaks of 1 | | | |
| Butyl acetate | 1 | 1 | | | |
| Dichloromethane | 6 | 6 | | 6 * | 6 * e |
| Ethanol | 1 * | 1 * | A x | | |
| Ethyl acetate | 1 | 1 | A; Some | | |
| | | | peaks of 1 | | |
| Diethyl ether | 1 * | 1 * | A x | | |
| Ethyl formate | 1 * | A; Some | | | 1 * e |
| | | peaks of 1 x | | | |
| Isopropyl acetate | 1 * | 1 * | A x | | |
| Methanol | 2 * | 2 * | A x | A e | |
| Methylisobutyl ketone | 1 | 1 | A x | | |
| N,N′-Dimethylacetamide | | | 1 * | | 1 |
| N,N′-Dimethylformamide | | | 1 | A e | 1 |
| N-Methylpyrrolidone | | | | | 1 * |
| Tetrahydrofuran | 1 | 1 | | | 1 * |
| Toluene | 7 | 8 | | | |
| Water | A | A | | | |
| Acetone:Water | | | 2 | 6 * e | 2 * |
| (90:10% v/v) | | | | | |
| Acetonitrile:Water | 6 | 6 | 6 | 6 * e | 2 * |
| (75:25% v/v) | | | | | |
| Ethanol:Water | A; Some | A; Some | A x | | |
| (50:50% v/v) | peaks of 2 | peaks of 2 | | | |
| 2-Propanol:Water | A; Some | A; Some | | | |
| (90:10% v/v) | peaks of 2 | peaks of 2 | | | |
| Tetrahydrofuran:Water | | | 1 * | | A |
| (98:2% v/v) | | | | | |
| Dichloromethane:Methanol | 2 | 2 | 2 * | 2 * | 2 * |
| (50:50% v/v) | | | | | |
| Dichloromethane:Methanol | | | 6 * | A e | A |
| (75:25% v/v) | | | | | |

empty—No solid;
A—Amorphous;
1—Free base Pattern 1;
2—Free base Pattern 2;
5—Free base Pattern 5;
6—Free base Pattern 6;
7—Free base Pattern 7;
8—Free base Pattern 8;
+—Additional peaks observed (likely due to presence of co-former);
*—Poorly crystalline;
x—Very little solid;
e—solids obtained by evaporation

Characterization of Free Base Forms

| | Solvent | | | ¹H | |
|---|---|---|---|---|---|
| Pattern | System | PLM | TG/DSC | NMR | Comment |
| 1 | 21 of the 34 | No clear | Melt onset | Consistent | Anhydrous; potential |
| | solvent | morphology; | 240° C.; <0.1 | with | thermodynamic form |
| | systems | appeared | equiv. of 2- | Compound | |
| | tested (via | weakly | ethoxyethanol | A | |
| | temperature | birefringent | (or 0.3 | | |
| | cycling, | | equiv. of | | |
| | evaporation | | water) | | |
| | and anti- | | released on | | |
| | solvent | | melting. | | |
| | addition) | | Decomposition | | |
| | | | above ~295° C. | | |
| 2 | Methanol | Distinct, | Loss of 0.3 | Consistent | Potential anhydrous |
| | (temperature | needle and | equiv. of | with | form containing |
| | cycling), | lath-like | MeOH, 0.1 | Compound | unbound solvent. |
| | acetone/water | particles; | equiv. of | A | Similar melt onset to |
| | (90:10 | appeared | DCM or 0.5 | | Pattern 1. Alternatively, |
| | % v/v) via | birefringent | equiv. of | | could possibly be a |
| | evaporation | | water below | | hemihydrate which |
| | and anti- | | 50° C. | | dehydrated to Pattern 1 |
| | solvent | | Decomposition | | |
| | addition, | | onset ~314° C. | | |
| | MeCN/water | | Melt onset | | |
| | (75:25% v/v) | | 243° C. | | |
| | by anti- | | | | |
| | solvent | | | | |
| | addition and | | | | |
| | DCM/MeOH | | | | |
| | (50:50% v/v) | | | | |
| | via all | | | | |
| | methods | | | | |
| 3 | 1,4-dioxane | Some rod- | 0.2 equiv. of | N/A | Potential 1,4-dioxane |
| | (converted | like | 1,4-dioxane | | solvate which de- |
| | from | particles | or 1 equiv. of | | solvated and |
| | amorphous | and | water lost up | | recrystallized to |
| | when dried). | agglomerates; | to 150° C. | | Pattern 1 |
| | Also | appeared | Loss of 0.6 | | |
| | observed | birefringent | equiv. of 1,4- | | |
| | during salt | | dioxane or | | |
| | screening | | 2.9 equiv. of | | |
| | with HCl, | | water | | |
| | glutamic | | associated | | |
| | acid, | | with a melt | | |
| | succinic acid | | (onset 178° C.) | | |
| | and acetic | | and | | |
| | acid; all in | | recrystallisation | | |
| | 1,4-dioxane | | event (onset | | |
| | | | 198° C.). | | |
| | | | Decomposition | | |
| | | | evident | | |
| | | | above ~302° C. | | |
| | | | Melt | | |
| | | | onset 236° C. | | |
| 4 | Observed | Some rod | Loss of 0.5 | N/A | Potential 1,4-dioxane |
| | during salt | and lath- | equiv. of 1,4- | | solvate which de- |
| | screening | like | dioxane or | | solvated and |
| | with malonic | particles | 2.6 equiv. of | | recrystallized to |
| | acid and | and | water | | Pattern 1 |
| | benzoic acid; | agglomerates; | between 60 | | |
| | both in 1,4- | appeared | and 150° C., | | |
| | dioxane | birefringent | immediately | | |
| | | | followed by a | | |
| | | | melt (onset | | |
| | | | 169° C.) and | | |
| | | | recrystallisation | | |
| | | | event | | |
| | | | (onset 192° C). | | |
| | | | Decomposition | | |
| | | | evident | | |
| | | | above ~302° C. | | |
| | | | Melt | | |
| | | | onset 235° C. | | |
| 5 | Anisole | — | — | — | Potential anisole solvate |
| | (converted to | | | | |
| | Pattern 8 on | | | | |
| | drying) | | | | |
| 6 | DCM | Some | Loss of 0.2 | Consistent | Unclear if anhydrous or |
| | (temperature | needle-like | equiv. of | with | hydrated. Similar melt |
| | cycling), , | particles | DCM or 1 | Compound | onset to Pattern 1. |
| | MeCN/water | and; | equiv. of | A. | Similar diffraction |
| | (75:25% v/v) | appeared | water up to | 2.4 wt. | pattern to Pattern 2. |
| | by | birefringent | 120° C. | % (0.23 | |
| | temperature | | Decomposition | eq.) | |
| | cycling and | | onset ~306° C. | ethyl | |
| | evaporation | | Melt onset | acetate | |
| | and | | 242° C. | | |
| | DCM/MeOH | | | | |
| | (75:25% v/v) | | | | |
| | via | | | | |
| | evaporation | | | | |
| 7 | Toluene | — | — | — | Potential toluene |
| | (converted to | | | | solvate. |
| | Pattern 8 on | | | | |
| | drying) | | | | |
| 8 | Drying of | No clear | 1 equiv. of | N/A | Potential anhydrous |
| | Patterns 5 | morphology; | anisole lost | | form produced on de- |
| | and 7 | appeared | below 100° C. | | solvation of anisole |
| | | weakly | (0.2 equiv. | | (Pattern 5) and toluene |
| | | birefringent | toluene in | | (Pattern 7) solvates. |
| | | | another | | Possibly recrystallized |
| | | | sample). | | to Pattern 1. |
| | | | Decomposition | | |
| | | | onset ~291° C. | | |
| | | | Recrystallisation | | |
| | | | onset 192° C. | | |
| | | | Melt | | |
| | | | onset 238° C. | | |
| 9 | 1,4-Dioxane | No clear | 8.2 wt. % (4 | — | Potential 1,4-dioxane |
| | from the co- | morphology; | equiv. of | | solvate. |
| | crystal | appeared | water or 0.8 | | |
| | screen | weakly | eq. 1,4- | | |
| | (Converted | birefringent. | dioxane) lost | | |
| | to Pattern 4 | Agglomeration | between 55° C. | | |
| | after storage | noted | and 175° C. | | |
| | at 40° C./75% | | 3.8 wt. % | | |
| | RH) | | (1.75 eq. | | |
| | | | water or 0.4 | | |
| | | | eq. 1.4- | | |
| | | | dioxane) lost | | |
| | | | between 255° C. | | |
| | | | and 290° C. | | |
| | | | Decomposition | | |
| | | | onset ~289° C. | | |
| | | | Exothermic | | |
| | | | event with | | |
| | | | onset: 146° C., | | |
| | | | peak: 164° C. | | |
| | | | Exothermic | | |
| | | | event with | | |
| | | | onset: 187° C., | | |
| | | | peak: 195° C. | | |
| | | | Endothermic | | |
| | | | event with | | |
| | | | onset: 238° C., | | |
| | | | peak: 243° C. | | |
| | | | Endothermic | | |
| | | | event with | | |
| | | | onset: 264° C., | | |
| | | | peak: 271° C. | | |

### Free Base Pattern 1

A 0.6% mass loss associated with melting was observed in the TG trace-likely due to the release of entrapped solvent upon melting. Decomposition noted above approximately 295° C. A sharp endothermic event was observed in the DSC trace (onset 240° C.) likely due to melting. See FIG. 39.
No clear morphology observed by PLM. Agglomerates were visible. The material appeared weakly birefringent under polarized light.
FT-IR analysis found that the spectra of free base Pattern 1 was very similar to the amorphous free base. A C—C triple bond peak was evident at 2214 cm⁻¹. Some potential THF peaks observed at higher wavenumbers. See FIG. 40.

### Free Base Pattern 2

TG/DSC analysis determined there was an initial 1.1% loss in mass in the TG trace from the onset of heating, likely due to unbound solvent was observed (0.3 eq. of methanol, 0.1 eq. of DCM, or 0.5 eq. of water). Decomposition was evident about approximately 314° C. A sharp endothermic event due to melting was observed in the DSC trace with an onset of 243° C. The melt occurred at a similar temperature to Pattern 1. See FIG. 41.
PLM analysis determined the morphology of free base Pattern 2 consisted of needles and lath-like particles of various sizes. The material appeared birefringent under polarized light, indicative of a crystalline material.
FT-IR analysis found that the spectra of free base Pattern 2 was very similar to the amorphous free base. A C—C triple bond peak was evident at 2220 cm⁻¹. There was no clear evidence of solvent or water. See FIG. 42.

### Free Base Pattern 3

TG/DSC analysis determined there was a gradual mass loss of 2.3% from the onset of heating up to approximately 150° C. observed in the TG trace. This mass loss was likely a result of unbound solvent loss (0.2 or 1 eq. of 1,4-dioxane or water, respectively). A second stepped loss in mass (6.1%) was noted between 15° and 240° C. (0.6 or 2.9 eq. of 1,4-dioxane or water, respectively). In the DSC trace, a possible melt/recrystallisation event appeared to occur during this loss in mass with onsets of 178 and 198° C. Following the recrystallisation event, an endothermic event associated with melting (onset 236° C.) was observed—similar to Pattern 1. See FIG. 43.
PLM analysis determined that the morphology appeared to consist of rods and agglomerates. The material appeared birefringent under polarized light.
FT-IR analysis found that the spectra of free base Pattern 3 was very similar to the amorphous free base, however, some additional weak peaks were present at higher wavenumbers. Possibly due to the presence of 1,4-dioxane and/or water. A C—C triple bond peak was evident at 2222 cm⁻¹. See FIG. 44.

### Free Base Pattern 4

In the TG/DSC, a gradual mass loss of 1.0% was observed in the TG trace from the onset of heating, up to approximately 50° C.—likely a result of unbound solvent loss. A second stepped loss in mass of 5.4% was noted between 6° and 150° C. (0.5 or 2.6 eq. of 1,4-dioxane or water, respectively). This indicated that Pattern 4 was a possible dioxane solvate. After this potential de-solvation, a possible melt/recrystallisation event was observed in the DSC trace with onsets of 169 and 192° C. Following the recrystallisation event, an endothermic event associated with melting (onset 235° C.) was observed—similar to Pattern 1. See FIG. 45.
PLM analysis determined the morphology appeared to consist of rods, laths and agglomerates. The material appeared birefringent under polarized light.
FT-IR analysis found that the spectra of free base Pattern 4 was very similar to the amorphous free base, however, some additional weak peaks were present at higher wavenumbers possibly due to the presence of 1,4-dioxane and/or water. A C—C triple bond peak was evident at 2223 cm⁻¹. See FIG. 46.

### Free Base Pattern 6

TG/DSC analysis found a 2.1% mass loss in the TG trace from the outset up to approximately 120° C. (0.2 eq. of DCM or 1 eq. of water). Decomposition was observed above 306° C. In the DSC trace, a sharp endothermic event due to melting was observed (onset 242)°—similar to Pattern 1. Additionally, a 0.3% loss in mass was noted in the TG trace during melting-likely entrapped solvent. See FIG. 47.
PLM analysis found that the morphology consisted of needle-like particles and that some agglomerates were visible. The material appeared weakly birefringent under polarized light.
FT-IR analysis found that the spectra of free base Pattern 6 was very similar to the amorphous free base. A C—C triple bond peak was evident at 2221 cm⁻¹. See FIG. 48.

### Free Base Pattern 8

TG/DSC analysis of the dried sample from anisole found a 14% loss in mass from the onset of heating up to 100° C. (1.2 eq. of anisole-possible solvate). Decomposition was observed above 307° C. In the DSC trace, a series of weak thermal events were noted after the possible desolvation before a sharp endothermic event (onset 237° C.) was observed-similar to Pattern 1. See FIG. 49.
The anisole sample was further dried and then re-analyzed by TG/DSC. In this TG trace, a 12.4% loss in mass was noted from the onset of heating up to 100° C. (1.1 equiv. of anisole-possible solvate). Decomposition was observed above 291° C. In the DSC trace, a potential recrystallisation event (onset 192° C.) was noted post-desolvation and a sharp endothermic event (onset 238° C.) due to melting was observed. See FIG. 50.
TG/DSC analysis of the dried toluene sample found two mass losses of 1 and 1.1% from the onset of heating up to 125° C. (0.2 eq. of toluene). Decomposition was observed above 290° C. A potential recrystallisation event (onset 193° C.) was noted in the DSC trace after the solvent loss. Finally, a sharp endothermic event (onset 238° C.) due to melting was observed in the DSC trace and a small mass loss associated with melting was noted in the TG trace. See FIG. 51.
PLM analysis found no clear morphology and that agglomeration was present. The material appeared birefringent under polarized light.
FT-IR analysis found that the spectra of free base Pattern 8 from anisole was very similar to the amorphous free base, although some slight shifts were noted. A C—C triple bond peak was evident at 2220 cm⁻¹. Some peaks potentially due to anisole present just above 3000 cm⁻¹. See FIG. 52.
FT-IR analysis found that the spectra of free base Pattern 8 from toluene was very similar to the amorphous free base, although some shifts were noted. A C—C triple bond peak was evident at 2220 cm⁻¹. See FIG. 53.

### Example 9. Secondary Crystallization Screen of Compound A

### Scale Up of Free Base Patterns 1, 2, and 6

Ca. 250 mg samples of Compound A were weighed into three scintillation vials.
The samples were dissolved in the appropriate solvents and observations were noted, see the table below.
A magnetic stirrer bar was added to each sample and the solutions were temperature cycled between 5° C. and 40° C. at a heating rate of 0.1° C./min with a 2 hour hold at each temperature for a total of 72 hours.
After ca. 72 hours, observations were noted and the samples were uncapped and left to evaporate at ambient conditions for ca. 20 hours.
   See Table 31 for the experimental details and observations made during the first attempt at scaling up at free base Patterns 1, 2, and 6.

TABLE 31
Details of the first attempt at scaling up free base Patterns 1, 2, and 6

| | | | Observations | Observations | |
|---|---|---|---|---|---|
| | | | before | after | Observations |
| Target | | Volume | temperature | temperature | after |
| Pattern | Solvent | (mL) | cycling | cycling | evaporation |
| 1 | THF | 2.5 | Purple | Off-white | White solids |
| | | | solution | slurry | |
| 2 | DCM:Methanol | 2.5 | Purple | Purple slurry | Off-white |
| | (50:50% v/v) | | solution | | solids |
| 6 | DCM | 2.5 | Purple | Purple slurry | Off-white |
| | | | solution | | solids |

The correct forms were obtained, but the samples were less crystalline than desirable, therefore 1 mL of the appropriate solvent was added to each sample to re-slurry and improve the crystallinity of the solids.
The slurries were temperature cycled between ambient and 40° C. over 4 hour cycles in an incubator shaker.
After ca. 72 hours, observations were made and solids were isolated by centrifugation.
The solids were analyzed by XRPD and then gently dried in an oven at 40° C. for ca. 20 hours.
Once dry, the material was characterized by XRPD, TG/DSC and ¹H NMR.
Free base Pattern 1 was further characterized by DSC, DVS, and optical rotation.

See Table 32 for the experimental details and observations made during the first attempt at scaling up at free base Patterns 1, 2, and 6.

TABLE 32
Details of the second attempt at scaling up free base Patterns 1, 2, and 6

| | | | Observations | Observations | |
|---|---|---|---|---|---|
| | | | before | after | Observations |
| Target | | Volume | temperature | temperature | after |
| Pattern | Solvent | (mL) | cycling | cycling | centrifugation |
| 1 | THF | 1 | Off-white | Off-white | White solids |
| | | | slurry | slurry | |
| 2 | DCM:Methanol | 1 | Purple slurry | Purple slurry | Off-white |
| | (50:50% v/v) | | | | solids |
| 6 | DCM | 1 | Purple slurry | Purple slurry | Off-white |
| | | | | | solids |

### Characterization of Free Base Patterns 1, 2, and 6.

XRPD Analysis of the Dried Free Base Forms Determined the Forms were Retained

### Free Base Pattern 1

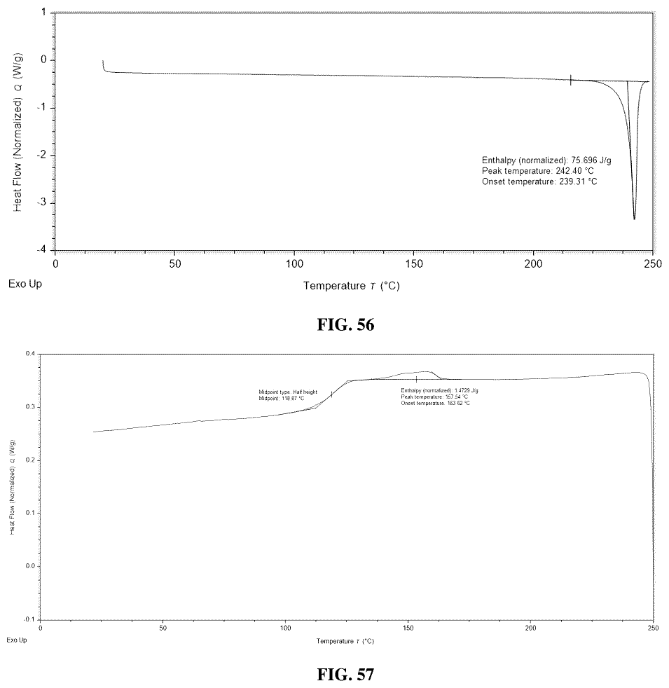

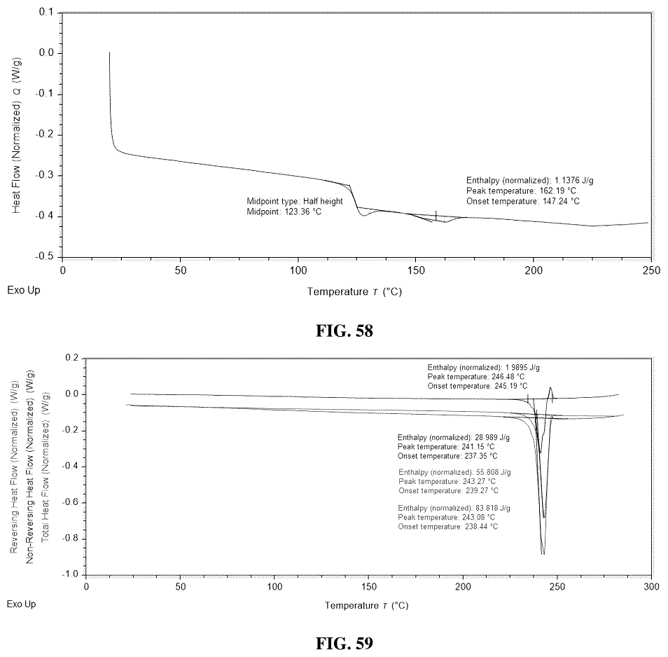

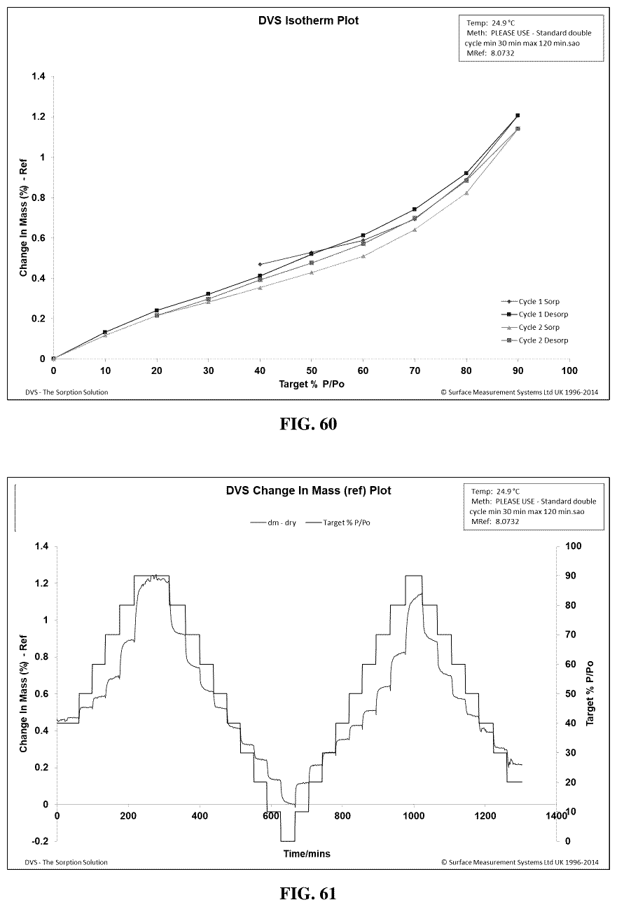

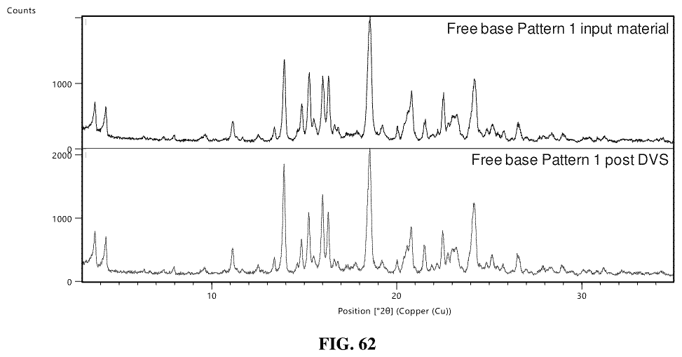

PLM analysis found small particles with a needle-like morphology. Agglomeration was noted. The material appeared slightly birefringent under polarized light.
By TG/DSC, no notable losses in mass were observed in the TG trace prior to decomposition (above 310° C.). An endothermic event due melting was noted (onset 239° C.) in the DSC trace. See FIG. 54.
The ¹H NMR spectrum was consistent with the structure. 0.32 wt. % (0.04 eq.) of THF was present in the sample. See FIG. 55. No peak shifting was observed compared to the ¹H NMR of Compound A.
DSC analysis of free base Pattern 1 found a sharp endothermic event in the first heating cycle with an onset at 239° C. and a peak at 242° C., this was due to the melt of the material. See FIG. 56.
In the cooling cycle, an exothermic event was observed with an onset at 164° C. and peak at 158° C. A glass transition with midpoint at 119° C. was also observed. See FIG. 57.
   The exothermic event was also observed in DSC analysis of the 3 g batch of Compound A Pattern 1 and was therefore confirmed to be intrinsic to the form.
The second heating cycle found a glass transition with a midpoint at 123° C. and a broad endothermic event with an onset at 147° C. See FIG. 58.
   The endothermic event was also observed in DSC analysis of the 3 g batch of Compound A Pattern 1 and was therefore confirmed to be intrinsic to the form. See FIGS. 83-85.
Modulated DSC analysis was carried out on the 3 g batch of Compound A Pattern 1. It showed that the melting endothermic event was present in the reversing and non-reversing heat flow with slightly different onsets at 239° C. and 237° C. respectively. Additionally, an exothermic event was observed in the non-reversing heat flow with an onset at 245° C., likely due to decomposition. (FIG. 59)
DVS analysis of free base Pattern 1 determined the material was slightly hygroscopic with a mass uptake of 1.17 wt. % (0.53 eq.) of water. The isotherm plot was a type 1 isotherm. See FIG. 60. No form change was observed in the kinetic plot. See FIG. 61. XRPD analysis confirmed that there was no form change post DVS. See FIG. 62.
For optical rotation, the samples and blanks were prepared with DCM. Optical rotation yielded a relative rotation [α]_{598 nm}^{19.5° C.} of −11.460° at 0.00969 g/mL in DCM for free base Pattern 1.

### Free Base Pattern 2

PLM analysis found small particles with an unclear morphology. Agglomeration was noted. The material appeared birefringent under polarized light.
TG/DSC of free base Pattern 2 found a small mass loss of 0.97 wt. % at the start of the experiment in the TG trace, likely due to surface moisture. Decomposition was noted above 305° C. In the DSC trace, a small exothermic event was observed (onset. 222° C.). Followed by an endothermic event due melting (onset 242° C.).
The ¹H NMR spectrum was consistent with the structure. No residual solvent was observed in the sample. No peak shifting was observed compared to the ¹H NMR of Compound A.

### Free Base Pattern 6

PLM analysis found very small particles with unclear morphology. Agglomeration was noted. The material was weakly birefringent under polarized light.
TG/DSC analysis found a small mass loss in the TG trace of 1.3 wt. % at the start of the experiment, likely due to surface moisture. Decomposition was noted above 305° C. In the DSC trace, an endothermic event due melting was noted (onset 241° C.). See FIG. 65.
The ¹H NMR spectrum was consistent with the structure. 0.67 wt. % (0.08 eq.) of THF was present in the sample. See FIG. 66. No peak shifting was observed compared to the ¹H NMR of Compound A.

### Competitive Slurry Experiments

Eight samples were prepared containing 10 mg of free base Patterns 1, 2, and 6.
The appropriate solvent systems were added and observations were noted.
After shaking at either ambient or 40° C., observations were noted and the samples were filtered centrifugally. The solids obtained were analyzed by XRPD.

See Table 33 for experimental details and observations.

TABLE 33
Details of the competitive slurry experiments

| | Solvent | Volume | Observations | | Observations |
|---|---|---|---|---|---|
| Number | System | (μL) | before shaking | Temperature | after shaking |
| 1 | THF:Heptane | 200 | Purple slurry | Ambient | Off-white slurry |
| | (50:50% v/v) | | | | |
| 2 | DCM:Methanol | 200 | Purple slurry | | Off-white slurry |
| | (20:80% v/v) | | | | |
| 3 | DCM:Heptane | 200 | Purple slurry | | Off-white slurry |
| | (50:50% v/v) | | | | |
| 4 | Water | 200 | Purple slurry | | Off-white slurry |
| 5 | THF:Heptane | 200 | Purple slurry | 40° C. | Off-white slurry |
| | (50:50% v/v) | | | | |
| 6 | DCM:Methanol | 200 | Purple slurry | | Off-white slurry |
| | (20:80% v/v) | | | | |
| 7 | DCM:Heptane | 200 | Purple slurry | | Off-white slurry |
| | (50:50% v/v) | | | | |
| 8 | Water | 200 | Purple slurry | | Off-white slurry |

Due to inconclusive results, the solids were returned to the sample vials and 100 μL of the appropriate solvent was added to each sample. The samples were then capped and sealed by parafilm and shaken at ambient or 40° C. as before.
After shaking for 72 hours, the solids were analyzed by XRPD.

### Competitive Slurry in Ethanol

An additional competitive slurry experiment was set up by combining ca. 5 mg each of Compound A Patterns 1, 2, and 6.
200 μL of ethanol was added to obtain an off-white slurry.
The sample was shaken at 40° C. for ca. 72 hours.
After 72 hours, an off-white slurry was obtained which was filtered centrifugally.
The solids were analyzed by XRPD.
The competitive slurry in ethanol yielded free base Pattern 1

### Competitive Slurries in DCM/Ethanol.

A 1:1 mixture of 5 mg of Compound A Patterns 1 and 2 was prepared in a 2 mL screw cap sample vial.
A mixture of 1 mg of Compound A Pattern 1 and 4 mg of Compound A Pattern 2 was prepared in a 2 mL screw cap sample vial.
   There was insufficient Pattern 1 material remaining from the scale up so material was recovered from the primary crystallization screen and dried at 40° C. for ca. 3 hours.
500 μL of 5% DCM in ethanol was added to the 1:1 mixture and 500 μL of 10% DCM in ethanol was added to the 1:4 mixture to form slurries.
   Both samples were capped, sealed in parafilm and shaken at ambient over the weekend.
   After ca. 72 hours, the solids were isolated centrifugally at 7500 rpm for 60 s and analyzed by XRPD.
   The sample at 5% DCM in ethanol yielded Compound A Pattern 1 and the sample at 10% DCM in ethanol yielded Compound A Pattern 2.
   Free base Pattern 1 was determined to be the thermodynamic form, however, DCM showed a preference to free base Pattern 2.

TABLE 34
Summary table of XRPD results of the first
set of competitive slurry experiments

| | Solvent | | |
|---|---|---|---|
| Number | System | Temperature | XRPD |
| 1 | THF:Heptane | Ambient | Pattern 1 |
| | (50:50% v/v) | | |
| 2 | DCM:Methanol | | Pattern 2 |
| | (20:80% v/v) | | |
| 3 | DCM:Heptane | | Mix of Patterns 1 and 2 |
| | (50:50% v/v) | | |
| 4 | Water | | Mix of Patterns 1 and 2 |
| 5 | THF:Heptane | 40° C. | Pattern 1 |
| | (50:50% v/v) | | |
| 6 | DCM:Methanol | | Mix of Patterns 1 and 2 |
| | (20:80% v/v) | | |
| 7 | DCM:Heptane | | Pattern 2 |
| | (50:50% v/v) | | |
| 8 | Water | | Mix of Patterns 1 and 2 |

TABLE 35
Summary table of XRPD results of the second
set of competitive slurry experiments

| | Solvent | | |
|---|---|---|---|
| Number | System | Temperature | XRPD |
| 1 | THF:Heptane | Ambient | Pattern 1 |
| | (50:50% v/v) | | |
| 2 | DCM:Methanol | | Pattern 2 |
| | (20:80% v/v) | | |
| 3 | DCM:Heptane | | Mix of Patterns 1 and 2 |
| | (50:50% v/v) | | |
| 4 | Water | | Pattern 1 |
| 5 | THF:Heptane | 40° C. | Pattern 1 |
| | (50:50% v/v) | | |
| 6 | DCM:Methanol | | Pattern 2 |
| | (20:80% v/v) | | |
| 7 | DCM:Heptane | | Mix of Patterns 1 and 2 |
| | (50:50% v/v) | | |
| 8 | Water | | Pattern 1 |

### Heating Experiment of Compound A Free Base Pattern 1

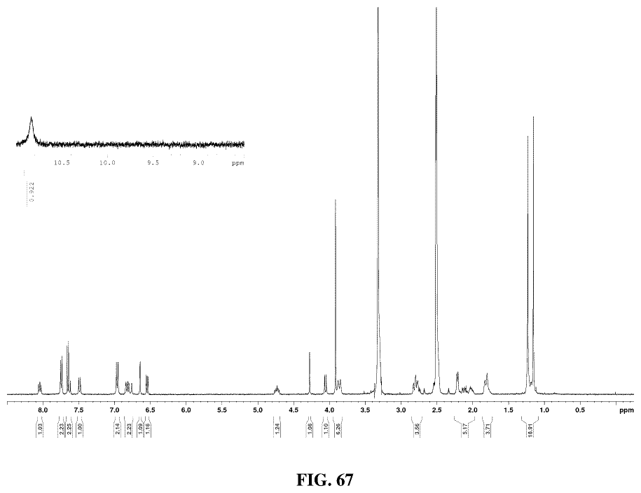

Ca. 4 mg of Compound A Pattern 1 was heated to 250° C. using the TG/DSC.
The sample was then analyzed by ¹H NMR.
No change was observed in the ¹H NMR spectrum after heating the sample to 250° C. This indicated that the sample had not degraded after being heating to this temperature. (FIG. 67)

TABLE 36
Characterisation of the scaled up free base Patterns 1, 2, and 6

| XRPD | | | | |
|---|---|---|---|---|
| Pattern | Solvent | PLM | TG/DSC | ¹H NMR |
| 1 | DCM | Small needle-like | No mass loss. | Consistent with |
| | | particles. | Decomposition | structure. |
| | | Agglomeration. | above | 0.32 wt. % |
| | | Weakly | 310° C. Melt | (0.04 eq.) of |
| | | birefringent. | onset 239° C. | THF |
| 2 | DCM: | Small particles, | No mass loss. | Consistent with |
| | methanol | unclear | Decomposition | structure. No |
| | | morphology. | above | residual solvent |
| | | Agglomeration. | 305° C. Melt | observed. |
| | | Birefringent. | onset 242° C. | |
| 6 | THF | Very small | No mass loss. | Consistent with |
| | | particles, unclear | Decomposition | structure. |
| | | morphology. | above | 0.67 wt. % |
| | | Agglomeration. | 305° C. Melt | (0.08 eq.) of |
| | | Weakly | onset 241° C. | THF |
| | | birefringent. | | |

TABLE 37
Full characterisation of free base Pattern 1

| | | Compound A Pattern 1 |
|---|---|---|
| PLM | | Very small birefringent particles, needle-shaped morphology < 5 μm in |
| | | length |
| ¹H NMR | | Consistent with structure. 0.32 wt. % (0.04 eq.) of THF |
| TG/DSC | | No mass losses, degradation above 310° C. Melt onset 239° C. |
| DSC | First heating cycle | Melt onset 239° C. (241° C.) |
| | Cooling cycle | Broad exothermic event with onset 164° C. followed by a glass |
| | | transition with midpoint 119° C. |
| | | (Sharp exothermic event with onset 168° C. followed by glass transition |
| | | with midpoint 118° C.) |
| | Second heating | Glass transition with midpoint 123° C. followed by a broad |
| | cycle | endothermic event with onset 147° C. |
| | | (Glass transition with midpoint at 123° C. followed by an endothermic |
| | | event with onset 166° C. and shoulder with peak 156° C.) |

| | |
|---|---|
| DVS | Slightly hygroscopic 1.17 wt. % (0.53 eq.) of water at 90% RH. No |
| | form change |
| Optical rotation | [α] value of −11.460° C. at 19.5° C., 598 nm |
| HPLC purity | 99.01% rel. area |

TABLE 38
Characterisation of the 3 g batch of free base Pattern 1

| | Compound A Pattern 1 (3 g batch) |
|---|---|
| PLM | Small birefringent particles (ca. 5 μm), unclear morphology |
| TG/DSC | No mass losses, degradation above 310° C. Melt onset at 240° C. |
| ¹H NMR | Consistent with structure. 0.6 wt. % (0.1 eq.) ethanol, 1.1 wt. % (0.1 |
| | eq.) IPA |
| Purity | 97.35% relative area |
| % Yield | 90.2% |

TABLE 39
Summary of the competitive slurry experiments

| Number | Solvent System | Temperature | XRPD |
|---|---|---|---|
| 1 | THF:Heptane | Ambient | Pattern 1 |
| | (50:50% v/v) | | |
| 2 | DCM:Methanol | | Pattern 2 |
| | (20:80% v/v) | | |
| 3 | DCM:Heptane | | Mix of patterns 1 and 2 |
| | (50:50% v/v) | | |
| 4 | Water | | Pattern 1 |
| 5 | THF: Heptane | 40° C. | Pattern 1 |
| | (50:50% v/v) | | |
| 6 | DCM:Methanol | | Pattern 2 |
| | (20:80% v/v) | | |
| 7 | DCM:Heptane | | Mix of patterns 1 and 2 |
| | (50:50% v/v) | | |
| 8 | Water | | Pattern 1 |
| 9 | Ethanol | | Pattern 1 |
| 10 | DCM:Ethanol | Ambient | Pattern 1 |
| | (5:95% v/v) | | |
| 11 | DCM:Ethanol | | Pattern 2 |
| | (10:90% v/v) | | |

### Example 10. Secondary Salt Screen of Compound A

### Scale Up

Three 250 mg samples of free base were dissolved in the appropriate solvent system to obtain clear purple solutions.
1.05 equivalents of the appropriate acids were added to each sample.
   See Table 40 for experimental details of the scale ups.

TABLE 40
Details of the secondary salt screen scale ups

| | Target | | Volume | |
|---|---|---|---|---|
| No. | salt | Solvent | (mL) | Acid |
| 1 | Tosylate | THF | 2.2 | 1.05 eq. p-Toluenesulfonic |
| | Pattern 1 | | | acid monohydrate in |
| | | | | 200 μL THF |
| 2 | Besylate | THF | 2.2 | 1.05 eq. Benzenesulfonic |
| | Pattern 1 | | | acid in 200 μL THF:Water |
| | | | | (98:2% v/v) |
| 3 | Phosphate | THF:Water | 2 | 1.05 eq of 1M Phosphoric |
| | Pattern 1 | (98:2% v/v) | | acid in THF |

The sample vials were capped and sealed in parafilm and then temperature cycled between ambient and 40° C. over 4 hour cycles for ca. 72 hours.
   Observations were noted before and after temperature cycling, see Table 41.

TABLE 41
Observations made during the scale up

| | | Observations | Observations | |
|---|---|---|---|---|
| | | before | after | Observations |
| | | temperature | temperature | after |
| No. | Target salt | cycling | cycling | drying |
| 1 | Tosylate | Off-white | Pale pink | Off-white |
| | Pattern 1 | slurry | slurry | solids |
| 2 | Besylate | Off-white | Red gel in | Red solids |
| | Pattern 1 | slurry | red solution | |
| 3 | Phosphate | Off-white | Light grey | Off-white |
| | Pattern 1 | slurry | slurry | solids |

The samples were filtered centrifugally, and the solids obtained from the slurries were analyzed by XRPD.
The solids and gel were dried under vacuum at 40° C. for ca. 20 hours.
The dried solids were analyzed by XRPD and submitted for NMR analysis.
The samples were re-slurried in 1.5 mL of the appropriate solvent system.
A further 1.05 eq. of the phosphoric acid stock solution was added to the phosphate sample.
The samples were then temperature cycled between ambient and 40° C. over 4-hour cycles for a total of 1 week.
After 1 week, the samples were isolated centrifugally and the solids obtained were analyzed by XRPD.

See Table 42 for details and observations made during the second attempt.

TABLE 42
Details of the second scale up attempt

| | | | | Observations | Observations | |
|---|---|---|---|---|---|---|
| | | | | before | after | Observations |
| | Target | | Volume | temperature | temperature | after |
| No. | salt | Solvent | (mL) | cycling | cycling | drying |
| 1 | Tosylate | THF | 1.5 | Pale pink | Pale pink | Off-white |
| | Pattern 1 | | | slurry | slurry | solids |
| 2 | Besylate | THF:Water | 1.5 | Red slurry | Light orange | Off-white |
| | Pattern 1 | (98:2 % | | | slurry | solids |
| | | v/v) | | | | |
| 3 | Phosphate | THF | 1.5 | Light grey | Light grey | Off-white |
| | Pattern 1 | | | slurry | slurry | solids |

The solids were then gently dried at 40° C. for ca. 20 hours.
The dried solids were analyzed by XRPD, multinuclear NMR, CAD (where applicable) and TG/DSC.
   NMR Analysis of the Material after the Initial Scale Up Attempt.
The ¹H NMR spectrum of the tosylate sample before the re-slurry determined there was a 1:1 ratio of p-toluenesulfonic acid to Compound A free base. 0.32 wt. % (0.04 eq.) of THF was present in the sample. Slight peak shifting was observed compared to the ¹H NMR of Compound A.
The ¹H NMR spectrum of the besylate sample was consistent with the structure, a 1:1 ratio of benzenesulfonic acid to Compound A free base was observed. 4.04 wt. % (0.5 eq.) THF was present in the sample. Slight peak shifting was observed compared to the ¹H NMR of Compound A.
The ¹H NMR spectrum of the phosphate sample was consistent with the structure. No solvent was observed in the sample. No phosphorus peaks were observed in the 31p NMR spectrum. No peak shifting was observed compared to the ¹H NMR of Compound A. This was not a phosphate salt, therefore additional phosphoric acid was added to the sample.

### Characterization of the Scaled Up Salts

### Tosylate Pattern 1

The scaled up sample matched the target pattern by XRPD.
PLM analysis found very small particles, the morphology was unclear due to the small particle size. Agglomeration was noted. The material appeared weakly birefringent under polarized light.
A 1:1 ratio of Compound A to p-toluenesulfonic acid was observed in the spectrum. Approximately 0.24 wt. % (0.03 eq.) of THF was present in the sample. See FIG. 68. Slight peak shifting was observed compared to the ¹H NMR of Compound A.
A 3.1 wt. % mass loss (1.23 eq. water or 0.31 eq. THF) was observed in the TG trace between ca. 90° C. and 220° C. Decomposition was noted above 280° C. An endothermic melting event with onset at 197.5° C. was observed in the DSC trace. See FIG. 69.

### Besylate Pattern 1

The scaled up sample matched the target pattern by XRPD.
PLM analysis found very small particles with an unclear morphology. Agglomeration was noted. The material appeared weakly birefringent under polarized light.
The ¹H NMR spectrum determined there was a 1:1 ratio of Compound A to benzenesulfonic acid. 3.02 wt. % (0.4 eq.) THF was observed in the sample. See FIG. 70. Slight peak shifting was observed compared to the ¹H NMR of Compound A.
A 0.9 wt. % mass loss (0.5 eq. water or 0.12 eq. THF) was observed in the TG trace from the onset of the experiment, likely surface moisture. A second 0.9 wt. % mass loss (0.5 eq. water or 0.12 eq. THF) was observed between 45° C. and 90° C. A third, larger mass loss of 1.45 wt. % (0.79 eq. water or 0.20 eq. THF) was observed between 100° C. and 175° C. Decomposition was noted above 210° C. Two endothermic events were noted in the DSC trace: the first with an onset at 184° C. (melt) and the second with an onset at 261° C. See FIG. 71.

### Phosphate Pattern 1

The scaled up sample matched the target pattern by XRPD.
PLM analysis found very small particles with an unclear morphology. Agglomeration was noted. The material appeared weakly birefringent under polarized light.
The ¹H NMR spectrum was consistent with the structure and contained a broad water peak indicative of salt formation. No residual solvent was observed in the sample. See FIG. 72. No peak shifting was observed compared to the ¹H NMR of Compound A. A phosphorus peak was observed in the ³¹P NMR spectrum, see FIG. 73.
A 0.5 wt. % mass loss (0.24 eq. water or 0.06 eq. THF) noted in the TG trace from the onset of the experiment. Another 6.5 wt. % mass loss (3.5 eq. of water or 0.9 eq. of THF) was observed between 70° C. and 160° C. Decomposition was noted above 270° C. A broad endothermic event was observed in the DSC trace related to the mass loss between 50° C. and 160° C. See FIG. 74.
CAD analysis found the presence of 22.2% w/w of phosphate, this corresponds to ca. 2 eq. of phosphate (1 eq.=10.8 wt. %, 2 eq.=19.5 wt. %)

TABLE 43
Characterisation carried out on the scaled up salts

| XRPD | | | | |
|---|---|---|---|---|
| Pattern | Solvent | PLM | TG/DSC | ¹H NMR |
| Tosylate | THF | Very small | 3.1 wt. % mass loss (1.23 eq. | 1:1 acid to free base. |
| Pattern 1 | | particles, | water or 0.31 eq. THF) from | Slight peak shifting. |
| | | unclear | onset. Decomposition above | 0.24 wt. % (0.03 eq.) |
| | | morphology. | 280° C. Melt onset: 197.5° C. | of THF |
| | | Agglomeration. | | |
| | | Weakly | | |
| | | birefringent. | | |
| Besylate | THF:water | Very small | 0.9 wt. % mass loss (0.5 eq. | 1:1 acid to free base. |
| Pattern 1 | | particles, | water or 0.12 eq. THF) from | Slight peak shifting. |
| | | unclear | onset, 0.9 wt. % mass loss (0.5 | 3.02 wt. % (0.4 eq.) |
| | | morphology. | eq. water or 0.12 eq. THF) | of THF |
| | | Agglomeration. | between 45° C.-90° C., 1.45 wt. | |
| | | Weakly | % mass loss (0.79 eq. water or | |
| | | birefringent. | 0.2 eq. THF) between 100° C.- | |
| | | | 175° C. Two endothermic | |
| | | | events; first onset at 184° C., | |
| | | | second onset at 261° C. | |
| Phosphate | THF | Very small | 0.47 wt. % mass loss (0.24 eq | Broad water peak. |
| Pattern 1 | | particles, | water or 0.06 eq. THF) from | Phosphate detected |
| | | unclear | onset, 6.46 wt. % mass loss | in the ³¹P |
| | | morphology. | (3.5 eq. water, 0.87 eq. THF, or | |
| | | Agglomeration. | 0.64 eq. phosphoric acid) | |
| | | Weakly | between 70° C. and 160° C. | |
| | | birefringent. | Decomposition above 270° C. | |
| | | | Broad endothermic event | |
| | | | observed during the second | |
| | | | mass loss. | |

### Example 11. Thermodynamic Solubility Determination

The following procedure was used to determine the solubility of Compound A free base Pattern 1, Compound A tosylate, Compound A besylate and Compound A phosphate.
Approximately 5 mg of the selected Compound A form was weighed into a clear glass vial and 2 mL of the buffer added.
In each case, a slurry persisted.
The pH of the solution was determined, then adjusted where necessary.
The slurries were agitated at 25° C.
At each timepoint, an aliquot was isolated and filtered through a 0.22 μm nylon filter, then injected without dilution into the HPLC.
The LOQ for the HPLC method was determined to be 0.00025 mg/mL.
All data were reported cf concentration of Compound A free base.
The solubility was unfortunately too low to be detected by HPLC for all samples at pH 3.0 and pH 4.0. These were reported to have a solubility<0.00025 mg/mL which was the LOQ for the experiment.
Free base Pattern 1 had the highest solubility of 0.0693 mg/mL after 24 hours at pH 1.2.
Prom the salts, the phosphate had the highest solubility of 0.0306 mg/mL after 24 hours at pH 1.2. No clear solubility advantage of the salts was noted over Compound A free base.

See Table 44 for a full summary of the thermodynamic solubility determination results.

TABLE 44
Results summary of the thermodynamic solubility determination

| | | 0 Hr pH | 0 Hr | 4 Hr pH | 4 Hr | 24 Hr pH | 24 Hr |
|---|---|---|---|---|---|---|---|
| | | (Adjusted | Solubility | (Adjusted | Solubility | (Adjusted | Solubility |
| Analyte | Buffer | pH) | (mg/mL) | pH) | (mg/mL) | pH) | (mg/mL) |
| Compound | pH | 1.26 | 0.0001 | 1.35 | 0.0262 | 1.28 | 0.0693 |
| A Free | 1.2 | | | (1.23) | | | |
| Base | pH | 2.92 | <0.00025 | 3 | <0.00025 | 2.93 | <0.00025 |
| Pattern 1 | 3.0 | | (<LOQ) | | (<LOQ) | | (<LOQ) |
| | pH | 3.99 | <0.00025 | 4.07 | <0.00025 | 3.93 | <0.00025 |
| | 4.0 | | (<LOQ) | | (<LOQ) | | (<LOQ) |
| Compound A | pH | 1.29 | 0.0122 | 1.27 | 0.0154 | 1.27 | 0.0063 |
| Tosylate | 1.2 | | | | | | |
| | pH | 2.94 | <0.00025 | 2.99 | <0.00025 | 2.96 | <0.00025 |
| | 3.0 | | (<LOQ) | | (<LOQ) | | (<LOQ) |
| | pH | 3.91 | <0.00025 | 3.92 | <0.00025 | 3.92 | <0.00025 |
| | 4.0 | | (<LOQ) | | (<LOQ) | | (<LOQ) |
| Compound A | pH | 1.25 | 0.0059 | 1.32 | 0.0291 | 1.24 | 0.0306 |
| Phosphate | 1.2 | | | (1.24) | | | |
| | pH | 2.75 | <0.00025 | 2.91 | <0.00025 | 2.88 | <0.00025 |
| | 3.0 | (2.93) | (<LOQ) | | (<LOQ) | (3.04) | (<LOQ) |
| | pH | 3.70 | <0.00025 | 4.02 | <0.00025 | 4.03 | <0.00025 |
| | 4.0 | (3.94) | (<LOQ) | | (<LOQ) | | (<LOQ) |
| Compound A | pH | 1.24 | 0.0133 | 1.34 | 0.0073 | 1.17 | 0.0093 |
| Besylate | 1.2 | | | (1.23) | | | |
| | pH | 2.94 | <0.00025 | 2.94 | <0.00025 | 2.94 | <0.00025 |
| | 3.0 | | (<LOQ) | | (<LOQ) | | (<LOQ) |
| | pH | 3.84 | <0.00025 | 4 | <0.00025 | 4.05 | < 0.00025 |
| | 4.0 | (3.93) | (<LOQ) | | (<LOQ) | | (<LOQ) |

### Example 12.7—Day Stability Assessment

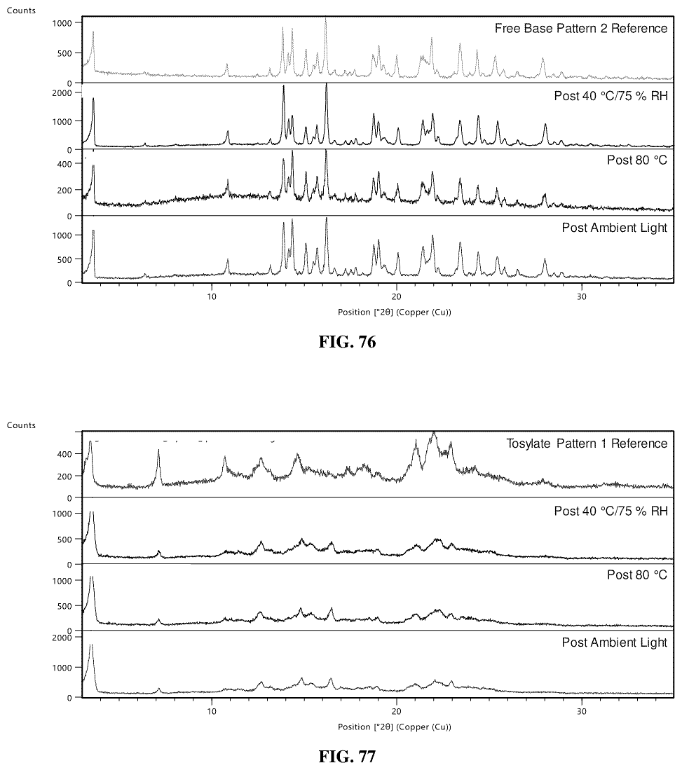

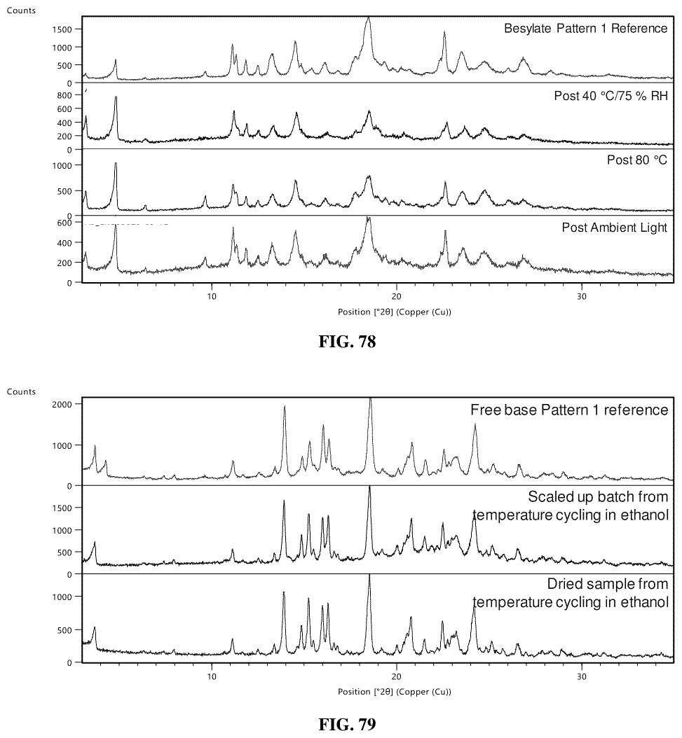

Three samples of 10-15 mg of the Compound A free base Patterns 1 and 2, as well as the Compound A tosylate and besylate salts were prepared. These were stored at the following stability conditions:
   40° C./75% RH (open vial)
   80° C. (closed vial)
   Ambient light (closed vial)
The appearance of the samples was noted daily to monitor color changes.
After 7 days, the samples were analyzed by HPLC and XRPD.
HPLC analysis was also carried out before stability testing.
The appearance of the samples did not change after 7 days at each stability condition. They all remained white or off-white solids.
XRPD analysis determined the patterns were retained. The tosylate salt samples gained an additional peak. See FIG. 75-78.
High purity was retained for all samples.
   >99% relative area for free base Pattern 1
   >98% relative area for free base Pattern 2
>96% by relative area for tosylate Pattern 1
>97% by relative area for besylate Pattern 1

See below for a summary of the results and for the impurity peak tables.

TABLE 45
Results summary of the 7-day stability assessment

| | | | | HPLC purity |
|---|---|---|---|---|
| | | Stability | | (% relative |
| No. | Sample | condition | XRPD | area) |
| 1 | Free base | N/A | FB Pattern 1 | 99.01 |
| 2 | Pattern 1 | 40° C./75% RH | No change | 99.00 |
| 3 | | 80° C. | No change | 99.03 |
| 4 | | Ambient light | No change | 99.07 |
| 5 | Tosylate | N/A | Tosylate | 97.44 |
| | salt | | Pattern 1 | |
| 6 | Pattern 1 | 40° C./75% RH | Additional peak | 97.30 |
| 7 | | 80° C. | Additional peak | 96.76 |
| 8 | | Ambient light | Additional peak | 97.09 |
| 9 | Besylate | N/A | Besylate | 97.33 |
| | Salt | | Pattern 1 | |
| 10 | Pattern 1 | 40° C./75% RH | No change | 97.31 |
| 11 | | 80° C. | No change | 97.56 |
| 12 | | Ambient light | No change | 97.17 |
| 13 | Free base | N/A | FB Pattern 2 | 98.39 |
| 14 | Pattern 2 | 40° C./75% RH | No change | 98.37 |
| 15 | | 80° C. | No change | 98.33 |
| 16 | | Ambient light | No change | 98.27 |

TABLE 46
Impurity peak table of free base Pattern 1

| | Retention | | | | Mean | Rel. | | Mean Rel. |
|---|---|---|---|---|---|---|---|---|
| Peak Name | time min | | RRT | | RRT | Area % | | Area % |
| Unknown 1 | n.a. | 3.04 | n.a | 0.58 | n.a | n.a | 0.05 | 0.05 |
| Unknown 13 | 4.48 | 4.48 | 0.85 | 0.85 | 0.847 | 0.67 | 0.66 | 0.66 |
| Unknown 5 | 5.16 | 5.17 | 0.98 | 0.98 | 0.977 | 0.07 | 0.07 | 0.07 |
| Unknown 14 | 5.47 | 5.47 | 1.04 | 1.04 | 1.035 | 0.16 | 0.15 | 0.15 |
| Unknown 10 | 5.75 | 5.75 | 1.09 | 1.09 | 1.088 | 0.06 | 0.06 | 0.06 |

| | |
|---|---|
| Total impurities | 0.99 |
| Reported Purity | 99.01 |

TABLE 47
Impurity peak table of free base Pattern
1 after 7 days at 40° C./75% RH

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.02 | 3.02 | 0.57 | 0.57 | 0.571 | 0.04 | 0.04 | 0.04 |
| Unknown | 4.47 | 4.47 | 0.85 | 0.85 | 0.846 | 0.67 | 0.67 | 0.67 |
| Unknown 5 | 5.17 | 5.17 | 0.98 | 0.98 | 0.977 | 0.07 | 0.07 | 0.07 |
| Unknown 14 | 5.42 | 5.48 | 1.03 | 1.04 | 1.025 | 0.16 | 0.15 | 0.16 |
| Unknown 10 | 5.76 | 5.76 | 1.09 | 1.09 | 1.089 | 0.06 | 0.06 | 0.06 |

| | |
|---|---|
| Total | 1.00 |
| impurities | |
| Reported | 99.00 |
| Purity | |

TABLE 48
Impurity peak table of free base Pattern 1 after 7 days at 80° C.

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.03 | 3.02 | 0.57 | 0.57 | 0.571 | 0.04 | 0.04 | 0.04 |
| Unknown 13 | 4.49 | 4.48 | 0.85 | 0.85 | 0.847 | 0.70 | 0.67 | 0.68 |
| Unknown 5 | 5.18 | 5.17 | 0.98 | 0.98 | 0.977 | 0.07 | 0.06 | 0.06 |
| Unknown 14 | 5.47 | 5.47 | 1.03 | 1.03 | 1.033 | 0.12 | 0.12 | 0.12 |
| Unknown 10 | 5.76 | 5.75 | 1.09 | 1.09 | 1.087 | 0.06 | 0.06 | 0.06 |

| | |
|---|---|
| Total | 0.97 |
| impurities | |
| Reported | 99.03 |
| Purity | |

TABLE 49
Impurity peak table of free base Pattern 1 after 7 days at ambient

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 13 | 4.48 | 4.48 | 0.85 | 0.85 | 0.846 | 0.68 | 0.67 | 0.68 |
| Unknown 5 | 5.17 | 5.18 | 0.98 | 0.98 | 0.977 | 0.07 | 0.08 | 0.07 |
| Unknown 14 | 5.46 | 5.46 | 1.03 | 1.03 | 1.031 | 0.11 | 0.11 | 0.11 |
| Unknown 10 | 5.77 | 5.76 | 1.09 | 1.09 | 1.090 | 0.07 | 0.06 | 0.07 |

| | |
|---|---|
| Total | 0.93 |
| impurities | |
| Reported | 99.07 |
| Purity | |

TABLE 50
Impurity peak table of tosylate Pattern 1

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.03 | 3.02 | 0.57 | 0.57 | 0.572 | 0.10 | 0.10 | 0.10 |
| Unknown 2 | 3.44 | 3.44 | 0.65 | 0.65 | 0.651 | 0.05 | 0.05 | 0.05 |
| Unknown 13 | 4.48 | 4.49 | 0.85 | 0.85 | 0.846 | 0.76 | 0.76 | 0.76 |
| Unknown 3 | 4.98 | 4.98 | 0.94 | 0.94 | 0.940 | 0.07 | 0.07 | 0.07 |
| Unknown 4 | 5.12 | 5.12 | 0.97 | 0.97 | 0.967 | 0.10 | 0.10 | 0.10 |
| Unknown 5 | 5.17 | 5.17 | 0.98 | 0.98 | 0.977 | 0.06 | 0.06 | 0.06 |
| Unknown 6 | 5.21 | 5.21 | 0.98 | 0.98 | 0.984 | 0.09 | 0.08 | 0.08 |
| Unknown 7 | 5.41 | 5.41 | 1.02 | 1.02 | 1.021 | 0.15 | 0.13 | 0.14 |
| Unknown 14 | 5.48 | 5.47 | 1.03 | 1.03 | 1.035 | 0.86 | 0.89 | 0.87 |
| Unknown 17 | n.a. | 5.55 | n.a. | 1.05 | n.a. | n.a. | 0.06 | 0.06 |
| Unknown 10 | 5.76 | 5.76 | 1.09 | 1.09 | 1.089 | 0.08 | 0.08 | 0.08 |
| Unknown 11 | 6.02 | 6.01 | 1.14 | 1.14 | 1.137 | 0.13 | 0.13 | 0.13 |
| Unknown 12 | 6.11 | 6.10 | 1.15 | 1.15 | 1.154 | 0.06 | 0.06 | 0.06 |

| | |
|---|---|
| Total | 2.56 |
| impurities | |
| Reported | 97.44 |
| Purity | |

TABLE 51
Impurity peak table of tosylate Pattern
1 after 7 days at 40° C./75% RH

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.04 | 3.02 | 0.58 | 0.57 | 0.577 | 0.10 | 0.09 | 0.10 |
| Unknown 2 | 3.47 | 3.44 | 0.66 | 0.65 | 0.658 | 0.05 | 0.05 | 0.05 |
| Unknown 13 | 4.48 | 4.49 | 0.85 | 0.85 | 0.850 | 0.76 | 0.76 | 0.76 |
| Unknown 3 | 4.96 | 4.98 | 0.94 | 0.94 | 0.941 | 0.12 | 0.13 | 0.13 |
| Unknown 4 | 5.10 | 5.13 | 0.97 | 0.97 | 0.968 | 0.10 | 0.10 | 0.10 |
| Unknown 5 | 5.16 | 5.18 | 0.98 | 0.98 | 0.978 | 0.08 | 0.08 | 0.08 |
| Unknown 6 | 5.19 | 5.21 | 0.98 | 0.98 | 0.985 | 0.08 | 0.08 | 0.08 |
| Unknown 7 | 5.39 | 5.41 | 1.02 | 1.02 | 1.022 | 0.16 | 0.11 | 0.14 |
| Unknown | n.a. | 5.44 | n.a. | 1.03 | n.a. | n.a. | 0.14 | 0.14 |
| Unknown 14 | 5.46 | 5.48 | 1.03 | 1.03 | 1.035 | 0.80 | 0.49 | 0.65 |
| Unknown 9 | 5.65 | 5.68 | 1.07 | 1.07 | 1.072 | 0.07 | 0.33 | 0.20 |
| Unknown 10 | 5.74 | 5.77 | 1.09 | 1.09 | 1.088 | 0.08 | 0.07 | 0.08 |
| Unknown 11 | 5.99 | 6.02 | 1.14 | 1.14 | 1.135 | 0.13 | 0.12 | 0.13 |
| Unknown 12 | 6.08 | 6.11 | 1.15 | 1.15 | 1.153 | 0.08 | 0.07 | 0.07 |

| | |
|---|---|
| Total | 2.70 |
| impurities | |
| Reported | 97.30 |
| Purity | |

TABLE 52
Impurity peak table of tosylate Pattern 1 after 7 days at 80° C.

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.02 | 3.02 | 0.57 | 0.57 | 0.570 | 0.10 | 0.10 | 0.10 |
| Unknown 2 | 3.44 | 3.45 | 0.65 | 0.65 | 0.649 | 0.05 | 0.05 | 0.05 |
| Unknown 13 | 4.49 | 4.50 | 0.85 | 0.85 | 0.848 | 0.66 | 0.66 | 0.66 |
| Unknown 3 | 4.98 | 4.99 | 0.94 | 0.94 | 0.941 | 0.19 | 0.18 | 0.18 |
| Unknown 4 | 5.13 | 5.13 | 0.97 | 0.97 | 0.968 | 0.11 | 0.12 | 0.12 |
| Unknown 5 | 5.17 | 5.19 | 0.98 | 0.98 | 0.977 | 0.08 | 0.08 | 0.08 |
| Unknown 6 | 5.21 | 5.22 | 0.98 | 0.98 | 0.984 | 0.12 | 0.11 | 0.11 |
| Unknown 7 | 5.41 | 5.42 | 1.02 | 1.02 | 1.021 | 0.30 | 0.31 | 0.31 |
| Unknown 14 | 5.48 | 5.49 | 1.03 | 1.03 | 1.034 | 1.0 | 0.98 | 1.00 |
| Unknown 17 | 5.55 | 5.56 | 1.05 | 1.05 | 1.048 | 0.08 | 0.08 | 0.08 |
| Unknown | 5.61 | 5.62 | 1.06 | 1.06 | 1.059 | 0.06 | 0.06 | 0.06 |
| Unknown 9 | 5.67 | 5.68 | 1.07 | 1.07 | 1.071 | 0.06 | 0.11 | 0.08 |
| Unknown 10 | 5.76 | 5.77 | 1.09 | 1.09 | 1.087 | 0.09 | 0.09 | 0.09 |
| Unknown | 5.83 | 5.83 | 1.10 | 1.10 | 1.100 | 0.09 | 0.08 | 0.08 |
| Unknown 11 | 6.01 | 6.02 | 1.13 | 1.13 | 1.134 | 0.17 | 0.16 | 0.16 |
| Unknown 12 | 6.10 | 6.11 | 1.15 | 1.15 | 1.151 | 0.08 | 0.08 | 0.08 |

| | |
|---|---|
| Total | 3.24 |
| impurities | |
| Reported | 96.76 |
| Purity | |

TABLE 53
Impurity peak table of tosylate Pattern 1 after 7 days at ambient

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.02 | 3.05 | 0.57 | 0.57 | 0.570 | 0.09 | 0.09 | 0.09 |
| Unknown 2 | 3.44 | 3.48 | 0.65 | 0.66 | 0.650 | 0.05 | 0.07 | 0.06 |
| Unknown 13 | 4.48 | 4.50 | 0.85 | 0.85 | 0.846 | 0.76 | 0.76 | 0.76 |
| Unknown 3 | 4.98 | 4.98 | 0.94 | 0.94 | 0.940 | 0.10 | 0.10 | 0.10 |
| Unknown 4 | 5.13 | 5.13 | 0.97 | 0.97 | 0.967 | 0.12 | 0.12 | 0.12 |
| Unknown 5 | 5.18 | 5.18 | 0.98 | 0.98 | 0.977 | 0.14 | 0.14 | 0.14 |
| Unknown 6 | 5.21 | 5.22 | 0.98 | 0.98 | 0.984 | 0.08 | 0.07 | 0.07 |
| Unknown | 5.42 | 5.42 | 1.02 | 1.02 | 1.023 | 0.16 | 0.16 | 0.16 |
| Unknown 14 | 5.49 | 5.48 | 1.04 | 1.03 | 1.035 | 1.1 | 1.1 | 1.06 |
| Unknown 9 | 5.68 | 5.68 | 1.07 | 1.07 | 1.072 | 0.07 | 0.07 | 0.07 |
| Unknown 10 | 5.77 | 5.77 | 1.09 | 1.09 | 1.089 | 0.09 | 0.09 | 0.09 |
| Unknown 11 | 6.02 | 6.02 | 1.14 | 1.13 | 1.136 | 0.14 | 0.14 | 0.14 |
| Unknown | 6.12 | 6.11 | 1.15 | 1.15 | 1.154 | 0.06 | 0.06 | 0.06 |

| | |
|---|---|
| Total | 2.91 |
| impurities | |
| Reported | 97.09 |
| Purity | |

TABLE 54
Impurity peak table of besylate Pattern 1

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 13 | 4.49 | 4.50 | 0.85 | 0.85 | 0.847 | 0.43 | 0.41 | 0.42 |
| Unknown 3 | 4.98 | 4.98 | 0.94 | 0.94 | 0.938 | 0.34 | 0.31 | 0.33 |
| Unknown 4 | 5.13 | 5.14 | 0.97 | 0.97 | 0.967 | 0.13 | 0.12 | 0.13 |
| Unknown 5 | 5.18 | 5.19 | 0.98 | 0.98 | 0.977 | 0.10 | 0.08 | 0.09 |
| Unknown 6 | 5.22 | 5.22 | 0.98 | 0.98 | 0.984 | 0.13 | 0.10 | 0.12 |
| Unknown | 5.42 | 5.43 | 1.02 | 1.02 | 1.022 | 0.22 | 0.20 | 0.21 |
| Unknown 14 | 5.49 | 5.49 | 1.03 | 1.03 | 1.034 | 0.68 | 0.65 | 0.67 |
| Unknown 17 | 5.56 | 5.56 | 1.05 | 1.05 | 1.048 | 0.12 | 0.11 | 0.12 |
| Unknown 9 | 5.68 | 5.68 | 1.07 | 1.07 | 1.070 | 0.07 | 0.06 | 0.07 |
| Unknown 11 | 6.02 | 6.02 | 1.14 | 1.13 | 1.135 | 0.16 | 0.15 | 0.15 |
| Unknown 15 | 6.41 | 6.40 | 1.21 | 1.21 | 1.207 | 0.09 | 0.06 | 0.08 |
| Unknown 16 | 6.45 | 6.45 | 1.22 | 1.21 | 1.215 | 0.08 | 0.07 | 0.08 |
| Unknown 17 | 6.51 | 6.50 | 1.23 | 1.22 | 1.227 | 0.25 | 0.22 | 0.23 |

| | |
|---|---|
| Total | 2.67 |
| impurities | |
| Reported | 97.33 |
| Purity | |

TABLE 55
Impurity peak table of besylate Pattern
1 after 7 days at 40° C./75% RH

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 13 | 4.49 | 4.50 | 0.85 | 0.85 | 0.847 | 0.41 | 0.42 | 0.42 |
| Unknown 3 | 4.96 | 4.97 | 0.94 | 0.94 | 0.936 | 0.32 | 0.32 | 0.32 |
| Unknown 4 | 5.12 | 5.14 | 0.97 | 0.97 | 0.967 | 0.13 | 0.13 | 0.13 |
| Unknown 5 | 5.20 | 5.19 | 0.98 | 0.98 | 0.981 | 0.11 | 0.11 | 0.11 |
| Unknown 6 | 5.23 | 5.22 | 0.99 | 0.98 | 0.987 | 0.15 | 0.09 | 0.12 |
| Unknown 7 | 5.40 | 5.43 | 1.02 | 1.02 | 1.019 | 0.19 | 0.30 | 0.24 |
| Unknown 14 | 5.48 | 5.50 | 1.03 | 1.04 | 1.035 | 0.67 | 0.66 | 0.67 |
| Unknown 17 | 5.55 | 5.56 | 1.05 | 1.05 | 1.047 | 0.10 | 0.12 | 0.11 |
| Unknown 9 | 5.67 | 5.68 | 1.07 | 1.07 | 1.070 | 0.05 | 0.07 | 0.06 |
| Unknown 11 | 6.02 | 6.02 | 1.14 | 1.13 | 1.135 | 0.14 | 0.14 | 0.14 |
| Unknown 15 | 6.40 | 6.40 | 1.21 | 1.21 | 1.208 | 0.07 | 0.08 | 0.07 |
| Unknown 16 | 6.44 | 6.44 | 1.22 | 1.21 | 1.215 | 0.08 | 0.08 | 0.08 |
| Unknown 17 | 6.50 | 6.50 | 1.23 | 1.22 | 1.226 | 0.20 | 0.22 | 0.21 |

| | |
|---|---|
| Total | 2.69 |
| impurities | |
| Reported | 97.31 |
| Purity | |

TABLE 56
Impurity peak table of besylate Pattern 1 after 7 days at 80° C.

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 13 | 4.51 | 4.51 | 0.85 | 0.85 | 0.852 | 0.40 | 0.41 | 0.41 |
| Unknown 3 | 4.97 | 4.97 | 0.94 | 0.94 | 0.938 | 0.30 | 0.31 | 0.30 |
| Unknown 4 | 5.13 | 5.14 | 0.97 | 0.97 | 0.969 | 0.13 | 0.13 | 0.13 |
| Unknown 5 | 5.19 | 5.20 | 0.98 | 0.98 | 0.979 | 0.09 | 0.09 | 0.09 |
| Unknown 6 | 5.21 | 5.23 | 0.98 | 0.98 | 0.984 | 0.09 | 0.09 | 0.09 |
| Unknown 7 | 5.41 | 5.43 | 1.02 | 1.02 | 1.021 | 0.20 | 0.16 | 0.18 |
| Unknown 14 | 5.48 | 5.50 | 1.03 | 1.03 | 1.033 | 0.66 | 0.64 | 0.65 |
| Unknown 17 | 5.53 | 5.55 | 1.04 | 1.04 | 1.043 | 0.09 | 0.10 | 0.10 |
| Unknown 9 | 5.65 | 5.67 | 1.07 | 1.07 | 1.066 | 0.05 | 0.09 | 0.07 |
| Unknown 11 | 5.98 | 6.01 | 1.13 | 1.13 | 1.129 | 0.10 | 0.10 | 0.10 |
| Unknown 15 | 6.37 | 6.39 | 1.20 | 1.20 | 1.201 | 0.06 | 0.06 | 0.06 |
| Unknown 16 | 6.41 | 6.43 | 1.21 | 1.21 | 1.209 | 0.06 | 0.06 | 0.06 |
| Unknown 17 | 6.47 | 6.49 | 1.22 | 1.22 | 1.220 | 0.21 | 0.18 | 0.20 |

| | |
|---|---|
| Total | 2.44 |
| impurities | |
| Reported | 97.56 |
| Purity | |

TABLE 57
Impurity peak table of besylate Pattern 1 after 7 days at ambient

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 13 | 4.50 | 4.51 | 0.85 | 0.85 | 0.848 | 0.42 | 0.43 | 0.43 |
| Unknown 3 | 4.96 | 4.97 | 0.93 | 0.93 | 0.934 | 0.37 | 0.37 | 0.37 |
| Unknown 4 | 5.13 | 5.14 | 0.97 | 0.97 | 0.967 | 0.14 | 0.14 | 0.14 |
| Unknown 6 | 5.19 | 5.21 | 0.98 | 0.98 | 0.979 | 0.14 | 0.15 | 0.14 |
| Unknown | 5.43 | 5.44 | 1.02 | 1.02 | 1.023 | 0.14 | 0.18 | 0.16 |
| Manual | 5.46 | 5.47 | 1.03 | 1.03 | 1.028 | 0.13 | 0.16 | 0.15 |
| identification | | | | | | | | |
| Unknown 14 | 5.49 | 5.52 | 1.04 | 1.04 | 1.035 | 0.60 | 0.64 | 0.62 |
| Unknown 17 | 5.54 | 5.57 | 1.04 | 1.05 | 1.045 | 0.11 | 0.12 | 0.11 |
| Unknown 9 | 5.67 | 5.70 | 1.07 | 1.07 | 1.068 | 0.06 | 0.07 | 0.06 |
| Unknown 10 | 5.78 | n.a. | 1.09 | n.a. | 1.089 | 0.05 | n.a. | 0.05 |
| Unknown 11 | 6.01 | 6.03 | 1.13 | 1.13 | 1.133 | 0.15 | 0.15 | 0.15 |
| Unknown 15 | 6.39 | 6.40 | 1.20 | 1.20 | 1.205 | 0.08 | 0.11 | 0.10 |
| Unknown 16 | 6.43 | 6.44 | 1.21 | 1.21 | 1.212 | 0.08 | 0.09 | 0.08 |
| Unknown 17 | 6.49 | 6.50 | 1.22 | 1.22 | 1.224 | 0.25 | 0.27 | 0.26 |

| | |
|---|---|
| Total | 2.83 |
| impurities | |
| Reported | 97.17 |
| Purity | |

TABLE 58
Impurity peak table of free base Pattern 2

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.03 | 3.04 | 0.57 | 0.57 | 0.570 | 0.08 | 0.07 | 0.07 |
| Unknown 13 | 4.50 | 4.52 | 0.85 | 0.85 | 0.846 | 0.64 | 0.66 | 0.65 |
| Unknown 4 | 5.14 | 5.15 | 0.97 | 0.97 | 0.967 | 0.08 | 0.08 | 0.08 |
| Unknown 6 | 5.23 | 5.24 | 0.98 | 0.98 | 0.984 | 0.10 | 0.12 | 0.11 |
| Unknown 14 | 5.51 | 5.51 | 1.04 | 1.04 | 1.036 | 0.45 | 0.54 | 0.50 |
| Unknown 9 | 5.68 | 5.67 | 1.07 | 1.07 | 1.068 | 0.10 | 0.07 | 0.09 |
| Unknown 10 | n.a. | 5.79 | n.a. | 1.09 | n.a. | n.a. | 0.05 | 0.05 |
| Unknown | 6.91 | 6.93 | 1.30 | 1.30 | 1.299 | 0.06 | 0.06 | 0.06 |

| | |
|---|---|
| Total | 1.61 |
| impurities | |
| Reported | 98.39 |
| Purity | |

TABLE 59
Impurity peak table of free base Pattern
2 after 7 days at 40° C./75% RH RRT

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.05 | 3.04 | 0.57 | 0.57 | 0.574 | 0.07 | 0.07 | 0.07 |
| Unknown 13 | 4.51 | 4.52 | 0.85 | 0.85 | 0.849 | 0.66 | 0.66 | 0.66 |
| Unknown 4 | 5.14 | 5.18 | 0.97 | 0.97 | 0.968 | 0.08 | 0.07 | 0.07 |
| Unknown 6 | 5.23 | 5.26 | 0.98 | 0.98 | 0.984 | 0.12 | 0.13 | 0.12 |
| Unknown 14 | 5.50 | 5.54 | 1.04 | 1.04 | 1.036 | 0.50 | 0.48 | 0.49 |
| Unknown 9 | 5.66 | 5.68 | 1.07 | 1.06 | 1.065 | 0.06 | 0.05 | 0.05 |
| Unknown 10 | 5.78 | 5.81 | 1.09 | 1.09 | 1.089 | 0.05 | 0.05 | 0.05 |
| Unknown 11 | n.a. | 6.01 | n.a. | 1.12 | n.a. | n.a. | 0.05 | 0.05 |
| Unknown | 6.93 | 6.92 | 1.30 | 1.29 | 1.304 | 0.06 | 0.06 | 0.06 |

| | |
|---|---|
| Total | 1.63 |
| impurities | |
| Reported | 98.37 |
| Purity | |

TABLE 60
Impurity peak table of free base Pattern 2 after 7 days at 80° C.

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.04 | 3.07 | 0.57 | 0.57 | 0.571 | 0.06 | 0.07 | 0.07 |
| Unknown 13 | 4.51 | 4.52 | 0.85 | 0.85 | 0.847 | 0.66 | 0.67 | 0.67 |
| Unknown 5 | 5.15 | 5.18 | 0.97 | 0.97 | 0.967 | 0.08 | 0.08 | 0.08 |
| Unknown | 5.24 | 5.27 | 0.98 | 0.98 | 0.984 | 0.13 | 0.14 | 0.13 |
| Unknown 17 | 5.52 | 5.54 | 1.04 | 1.04 | 1.036 | 0.51 | 0.52 | 0.51 |
| Unknown 9 | 5.67 | 5.67 | 1.07 | 1.06 | 1.065 | 0.06 | 0.06 | 0.06 |
| Unknown 10 | 5.80 | 5.80 | 1.09 | 1.08 | 1.089 | 0.05 | 0.05 | 0.05 |
| Unknown 11 | n.a. | 5.99 | n.a. | 1.12 | n.a. | n.a. | 0.05 | 0.05 |
| Unknown | 6.93 | 6.92 | 1.30 | 1.29 | 1.301 | 0.06 | 0.06 | 0.06 |

| | |
|---|---|
| Total | 1.67 |
| impurities | |
| Reported | 98.33 |
| Purity | |

TABLE 61
Impurity peak table of free base Pattern 2 after 7 days at ambient

| | | | | | | | | Mean |
|---|---|---|---|---|---|---|---|---|
| | Retention | | | | Mean | Rel. | | Rel. |
| Peak Name | time min | | RRT | | RRT | Area % | | Area % |
| Unknown 1 | 3.11 | 3.07 | 0.58 | 0.57 | 0.582 | 0.07 | 0.06 | 0.07 |
| Unknown | 4.54 | 4.54 | 0.85 | 0.85 | 0.850 | 0.65 | 0.65 | 0.65 |
| Unknown 5 | 5.17 | 5.18 | 0.97 | 0.97 | 0.968 | 0.08 | 0.08 | 0.08 |
| Unknown | 5.27 | 5.27 | 0.99 | 0.99 | 0.985 | 0.17 | 0.17 | 0.17 |
| Unknown 17 | 5.54 | 5.54 | 1.04 | 1.04 | 1.036 | 0.54 | 0.53 | 0.53 |
| Unknown 9 | 5.66 | 5.66 | 1.06 | 1.06 | 1.059 | 0.11 | 0.11 | 0.11 |
| Unknown | 5.82 | 5.82 | 1.09 | 1.09 | 1.089 | 0.06 | 0.06 | 0.06 |
| Unknown | 6.95 | 6.95 | 1.30 | 1.30 | 1.299 | 0.06 | 0.06 | 0.06 |

| | |
|---|---|
| Total | 1.73 |
| impurities | |
| Reported | 98.27 |
| Purity | |

### Example 13.3 G Scale Up of Compound a Pattern 1

Ca. 3 g of amorphous Compound A was added to a pre-weighed scintillation vial. 5 mL of THF was added and a purple slurry was obtained. A magnetic stirrer bar was added and the sample was temperature cycled between 20° C. and 40° C. at a heating rate of 0.1° C./min with a 2 hour hold at 20° C. and 40° C. The sample was checked after ca. 3 hours and a thick off-white slurry was noted. An additional 5 mL of THF was added to yield a movable slurry.
After ca. 72 hours, the sample was a thick off-white slurry. It was filtered over a Büchner funnel and allowed to dry on the filter bed for ca. 5 minutes. A sub-sample was analyzed by XRPD. It was found to be poorly crystalline Pattern 1. The sample was gently dried at 40° C. for ca. 20 hours and then re-analyzed by XRPD.
25 mg of Free base Pattern 1 seed material was added to the material and the sample was re-slurried in 7 mL of ethanol. The slurry was temperature cycled between ambient and 40° C. over 4 hour cycles for ca. 24 hours. A sub-sample was analyzed by XRPD and the material was then gently dried at 40° C. and re-analyzed by XRPD.
The poorly crystalline Pattern 1 sample was re-slurried in 10 mL of methanol:DCM (90:10% v/v) with 5 mg of free base Pattern 1 seed and temperature cycled between ambient and 40° C. over 4 hour cycles for ca. 72 hours.
A mix of free base Patterns 2 and 7 were obtained. The sample was filtered by Büchner filtration and then thoroughly dried at ambient under vacuum for ca. 3 hours.
The dried sample was re-slurried in 10 mL of ethanol with 3.5 mg of free base Pattern 1 seed and temperature cycled between ambient and 40° C. over 4 hour cycles for ca. 48 hours.
After 48 hours, a thick slurry was obtained and dried by Buchner filtration. A sub-sample was analyses by XRPD which confirmed the sample was successfully converted to Pattern 1.
The material was placed in a pre-weighed scintillation vial, covered in tissue paper and gently dried in a 40° C. oven for ca. 20 hours.
The dried material was analyzed by XRPD, PLM, TG/DSC, DSC, modulated DSC, ¹H NMR, and HPLC.

### Results

The sample from THE yielded poorly crystalline Pattern 1. This pattern was retained after re-slurrying with Pattern 1 seed in ethanol.
This sample was analyzed by PLM, very small weakly birefringent particles with unclear morphology were observed. The small particle size likely influenced the appearance of the XRPD pattern.
A mixture of free base Patterns 2 and 7 was obtained after temperature cycling in methanol:DCM (90:10% v/v) for ca. 72 hours.
Free base Pattern 1 was successfully scaled up by slurrying the free base Patterns 2 and 7 material in ethanol with Pattern 1 seed material.
The 3 g scale up of Compound A Pattern 1 yielded 2.68 g (90.2% yield).

### Characterization of the 3 g Scale Up of Compound A Pattern 1.

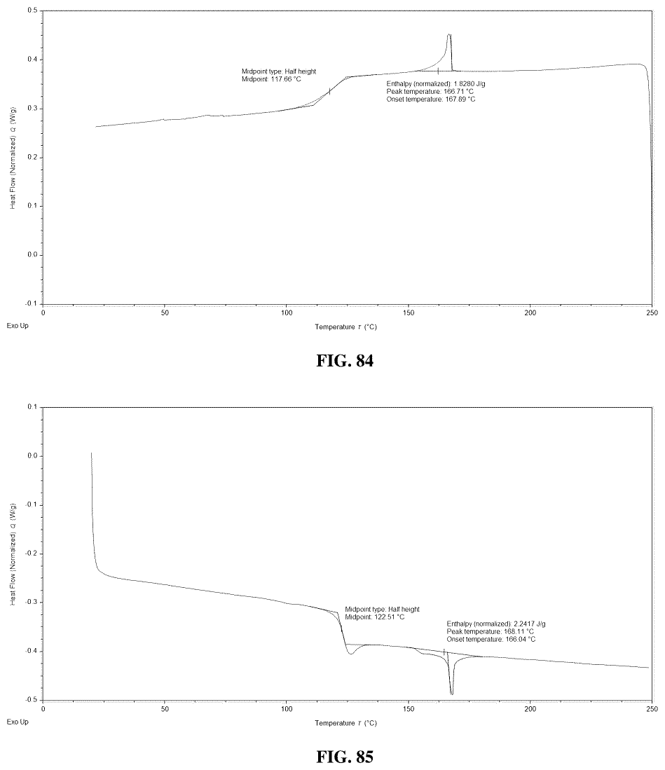

The XRPD pattern matched the previously found free base Pattern 1, see FIG. 80.
PLM analysis found small birefringent particles ca. 5 μm in size. The morphology was unclear.
The ¹H NMR spectrum was consistent with the free base spectrum. 0.6 wt. % (0.1 eq.) of ethanol and 1.1 wt. % (0.1 eq.) of IPA were observed in the spectrum. See FIG. 81. No peak shifting was observed compared to Compound A.
No mass losses were observed in the TG trace until degradation after ca. 310° C. A sharp endothermic event was observed in the DSC trace with an onset at 240° C. corresponding to the melt. See FIG. 82.
DSC analysis was carried out on this batch to confirm that the exothermic and endothermic events observed in the cooling and second heating cycles respectively were indicative of the form and not an artefact of that batch. A sharp endothermic melting event with onset 241° C. was observed in the first heating cycle, matching previous data. See FIG. 83.
In the DSC cooling cycle, an exothermic event was observed with an onset at 168° C. and peak 167° C. (previous batch had a broader event with onset at 164° C. and peak at 158° C.). A glass transition with midpoint at 118° C. (previously 119° C.) was also observed. See FIG. 84.
The second heating cycle found a glass transition with a midpoint at 123° C. (same temp. as previous) and an endothermic event with an onset at 166° C. and peak at 168° C., the event had a shoulder with the peak at 156° C. (Previously the onset was found at 147° C. and the peak at 162° C. See FIG. 85.
   The exo- and endothermic event were obtained in the DSC analysis of this batch as well as the previous batch and were thus confirmed to be due to the free base form. See FIGS. 56-58 for the previous batch.
HPLC analysis determined the purity of the batch was 97.35% by relative area.

### Equivalents

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

### Enumerated Embodiments

The aspects of the present disclosure are further described with reference to the following embodiments:
A. A solid form of Compound A:

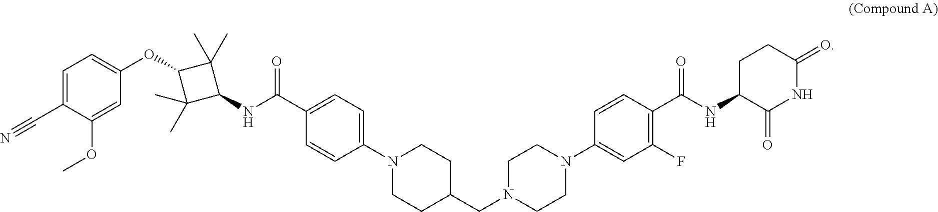 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

B. The solid form of embodiment A, wherein the solid form is crystalline.
C. The solid form of embodiment A, wherein the solid form is amorphous.
D. The solid form of any of the preceding embodiments, wherein the solid form is a crystalline polymorphic form characterized by two or three XRPD signals selected from the group consisting of 18.6 °2θ, 13.9 °2θ, and 15.3 °2θ (±0.2 °2θ; ±0.1 °2θ; or ±0.0 °2θ; Cu Kα1 radiation).
E. The solid form of any of the preceding embodiments, wherein the solid form is a crystalline polymorphic form characterized by XRPD signals at 18.6 °2θ, 13.9 °2θ, and 15.3 °2θ (±0.2 °2θ; ±0.1 °2θ; or ±0.0 °2θ; Cu Kα1 radiation).
F. The solid form of any of the preceding embodiments, wherein the solid form is a crystalline polymorphic form characterized by an XRPD spectrum substantially similar to that shown in FIG. 80 or FIG. 86.
G. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, or forty-one XRPD signals selected from those set forth in Table 1.
H. The solid form of any of the preceding embodiments, wherein the solid form is a crystalline polymorphic form characterized by two or three XRPD signals selected from the group consisting of 14.4 °2θ, 19.1 °2θ, and 15.8 °2θ (±0.2 °2θ; ±0.1 °2θ; or ±0.0 °2θ; Cu Kα1 radiation).
I. The solid form of any of the preceding embodiments, wherein the solid form is a crystalline polymorphic form characterized by XRPD signals at 14.4 °2θ, 19.1 °2θ, and 15.8 °2θ (±0.2 °2θ; ±0.1 °2θ; or ±0.0 °2θ; Cu Kα1 radiation).
J. The solid form of any of the preceding embodiments, wherein the solid form is a crystalline polymorphic form characterized by an XRPD spectrum substantially similar to that shown in FIG. 87.
K. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, or forty XRPD signals selected from those set forth in Table 2.
L. The solid form of any of the preceding embodiments, wherein the solid form is a crystalline polymorphic form characterized by two or more, or three or more XRPD signals selected from the group consisting of 20.6 °2θ, 16.1 °2θ, 16.3 °2θ, 17.3 °2θ, and 16.8 °2θ (±0.2 °2θ; ±0.1 °2θ; or ±0.0 °2θ; Cu Kα1 radiation).
M. The solid form of any of the preceding embodiments, wherein the solid form is a crystalline polymorphic form characterized by XRPD signals at 20.6 °2θ, 16.1 °2θ, 16.3 °2θ, 17.3 °2θ, and 16.8 °2θ (±0.2 °2θ; ±0.1 °2θ; or ±0.0 °2θ; Cu Kα1 radiation).
N. The solid form of any of the preceding embodiments, wherein the solid form is a crystalline polymorphic form characterized by an XRPD spectrum substantially similar to that shown in FIG. 88.
O. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, or nineteen XRPD signals selected from those set forth in Table 3.
P. The solid form of any of the preceding embodiments, wherein the solid form is a crystalline polymorphic form characterized by two or three XRPD signals selected from the group consisting of 14.6 °2θ, 17.7 °2θ, and 16.7 °2θ (±0.2 °2θ; ±0.1 °2θ; or ±0.0 °2θ; Cu Kα1 radiation).
Q. The solid form of any of the preceding embodiments, wherein the solid form is a crystalline polymorphic form characterized by XRPD signals at 14.6 °2θ, 17.7 °2θ, and 16.7 °2θ (±0.2 °2θ; ±0.1 °2θ; or ±0.0 °2θ; Cu Kα1 radiation).
R. The solid form of any of the preceding embodiments, wherein the solid form is a crystalline polymorphic form characterized by an XRPD spectrum substantially similar to that shown in FIG. 89.
S. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four XRPD signals selected from those set forth in Table 4.
T. The solid form of any of the preceding embodiments, wherein the solid form is a crystalline polymorphic form characterized by two or three XRPD signals selected from the group consisting of 14.9 °2θ, 22.6 °2θ, and 7.1 °2θ (±0.2 °2θ; ±0.1 °2θ; or ±0.0 °2θ; Cu Kα1 radiation).
U. The solid form of any of the preceding embodiments, wherein the solid form is a crystalline polymorphic form characterized by XRPD signals at 14.9 °2θ, 22.6 °2θ, and 7.1 °2θ (±0.2 °2θ; ±0.1 °2θ; or ±0.0 °2θ; Cu Kα1 radiation).
V. The solid form of any of the preceding embodiments, wherein the solid form is a crystalline polymorphic form characterized by an XRPD spectrum substantially similar to that shown in FIG. 90.
W. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, or thirty-five XRPD signals selected from those set forth in Table 5.
X. The solid form of any of the preceding embodiments, wherein the solid form is a crystalline polymorphic form characterized by two or three XRPD signals selected from the group consisting of 3.5 °2θ, 3.6 °2θ, and 15.7 °2θ (±0.2 °2θ; ±0.1 °2θ; or ±0.0 °2θ; Cu Kα1 radiation).
Y. The solid form of any of the preceding embodiments, wherein the solid form is a crystalline polymorphic form characterized by XRPD signals at 3.5 °2θ, 3.6 °2θ, and 15.7 °2θ (±0.2 °2θ; ±0.1 °2θ; or ±0.0 °2θ; Cu Kα1 radiation).
Z. The solid form of any of the preceding embodiments, wherein the solid form is a crystalline polymorphic form characterized by an XRPD spectrum substantially similar to that shown in FIG. 91.
AA. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, or thirty-six XRPD signals selected from those set forth in Table 6.
BB. The solid form of any of the preceding embodiments, wherein the solid form is a crystalline polymorphic form characterized by two or three XRPD signals selected from the group consisting of 4.8 °2θ, 15.7 °2θ, and 17.9 °2θ (±0.2 °2θ; ±0.1 °2θ; or ±0.0 °2θ; Cu Kα1 radiation).
CC. The solid form of any of the preceding embodiments, wherein the solid form is a crystalline polymorphic form characterized by XRPD signals at 4.8 °2θ, 15.7 °2θ, and 17.9 °2θ (±0.2 °2θ; ±0.1 °2θ; or ±0.0 °2θ; Cu Kα1 radiation).
DD. The solid form of any of the preceding embodiments, wherein the solid form is a crystalline polymorphic form characterized by an XRPD spectrum substantially similar to that shown in FIG. 92.
EE. The solid form of any of the preceding embodiments, wherein the solid form is a crystalline polymorphic form characterized by one, two, three, four, five, six, seven, eight, nine, ten, eleven, twelve, thirteen, fourteen, fifteen, or sixteen XRPD signals selected from those set forth in Table 7.
FF. The solid form of any of the preceding embodiments, wherein the solid form is a crystalline polymorphic form characterized by two or three XRPD signals selected from the group consisting of 4.9 °2θ, 15.9 °2θ, and 18.2 °2θ (±0.2 °2θ; ±0.1 °2θ; or ±0.0 °2θ; Cu Kα1 radiation).
GG. The solid form of any of the preceding embodiments, wherein the solid form is a crystalline polymorphic form characterized by XRPD signals at 4.9 °2θ, 15.9 °2θ, and 18.2 °2θ (±0.2 °2θ; ±0.1 °2θ; or ±0.0 °2θ; Cu Kα1 radiation).
HH. The solid form of any of the preceding embodiments, wherein the solid form is a crystalline polymorphic form characterized by an XRPD spectrum substantially similar to that shown in FIG. 93.
II. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, or twenty-two XRPD signals selected from those set forth in Table 8.
JJ. The solid form of any of the preceding embodiments, wherein the solid form is a crystalline polymorphic form characterized by two or three XRPD signals selected from the group consisting of 17.2 °2θ, 21.0 °2θ, and 24.2 °2θ (±0.2 °2θ; ±0.1 °2θ; or ±0.0 °2θ; Cu Kα1 radiation).
KK. The solid form of any of the preceding embodiments, wherein the solid form is a crystalline polymorphic form characterized by XRPD signals at 17.2 °2θ, 21.0 °2θ, and 24.2 °2θ (±0.2 °2θ; ±0.1 °2θ; or ±0.0 °2θ; Cu Kα1 radiation).
LL. The solid form of any of the preceding embodiments, wherein the solid form is a crystalline polymorphic form characterized by an XRPD spectrum substantially similar to that shown in FIG. 94.
MM. The solid form of any of the preceding embodiments, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, or forty-two XRPD signals selected from those set forth in Table 9.
NN. A tosylate salt of Compound A:

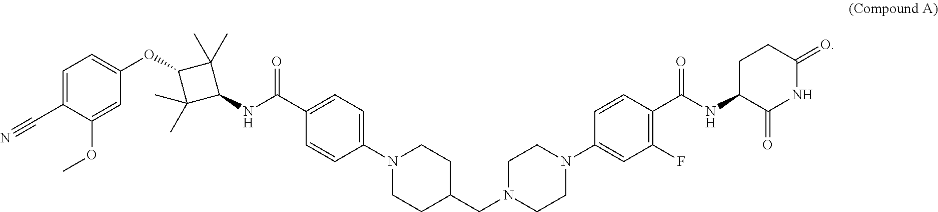 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

OO. The tosylate salt of embodiment 40, wherein the tosylate salt is crystalline.
PP. The tosylate salt of embodiment 40, wherein the tosylate salt is amorphous.
QQ. The tosylate salt of any of the preceding embodiments, wherein the tosylate salt is a crystalline polymorphic form characterized by two or three XRPD signals selected from the group consisting of 3.5 °2θ, 22.0 °2θ, and 23.0 °2θ (±0.2 °2θ; ±0.1 °2θ; or ±0.0 °2θ; Cu Kα1 radiation).
RR. The tosylate salt of any of the preceding embodiments, wherein the tosylate salt is a crystalline polymorphic form characterized by XRPD signals at 3.5 °2θ, 22.0 °2θ, and 23.0 °2θ (±0.2 °2θ; ±0.1 °2θ; or ±0.0 °2θ; Cu Kα1 radiation).
SS. The tosylate salt of any of the preceding embodiments, wherein the tosylate salt is a crystalline polymorphic form characterized by an XRPD spectrum substantially similar to that shown in FIG. 95.
TT. The tosylate salt of any of the preceding embodiments, wherein the tosylate salt is a crystalline polymorphic form characterized by one, two, three, four, five, six, seven, eight, nine, ten, eleven, twelve, or thirteen XRPD signals selected from those set forth in Table 10.
UU. The tosylate salt of any of the preceding embodiments, wherein the tosylate salt is a 1:1 tosylate:Compound A salt.
VV. The tosylate salt of any of the preceding embodiments, wherein the tosylate salt is a 2:1 tosylate:Compound A salt.
WW. The tosylate salt of any of the preceding embodiments, wherein the tosylate salt is a 1:2 tosylate:Compound A salt.
XX. A phosphate salt of Compound A:

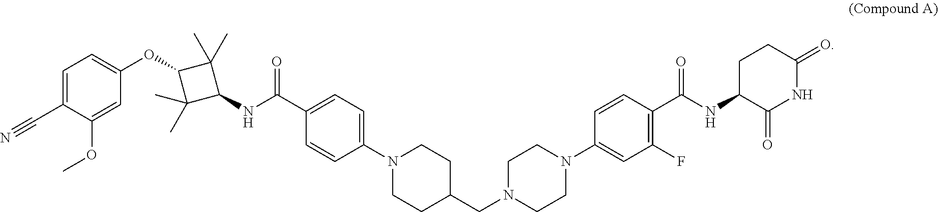 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

YY. The phosphate salt of embodiment 50, wherein the phosphate salt is crystalline.
ZZ. The phosphate salt of embodiment 50, wherein the phosphate salt is amorphous.
AAA. The phosphate salt of any of the preceding embodiments, wherein the phosphate salt is a crystalline polymorphic form characterized by two or three XRPD signals selected from the group consisting of 23.6 °2θ, 3.3 °2θ, and 19.9 °2θ (±0.2 °2θ; ±0.1 °2θ; or ±0.0 °2θ; Cu Kα₁ radiation).
BBB. The phosphate salt of any of the preceding embodiments, wherein the phosphate salt is a crystalline polymorphic form characterized by XRPD signals at 23.6 °2θ, 3.3 °2θ, and 19.9 °2θ (±0.2 °2θ; ±0.1 °2θ; or ±0.0 °2θ; Cu Kα1 radiation).
CCC. The phosphate salt of any of the preceding embodiments, wherein the phosphate salt is a crystalline polymorphic form characterized by an XRPD spectrum substantially similar to that shown in FIG. 96.
DDD. The phosphate salt of any of the preceding embodiments, wherein the phosphate salt is a crystalline polymorphic form characterized by one, two, three, four, five, six, seven, eight, nine, ten, eleven, twelve, thirteen, or fourteen XRPD signals selected from those set forth in Table 11.
EEE. The phosphate salt of any of the preceding embodiments, wherein the phosphate salt is a 1:1 phosphate:Compound A salt.
FFF. The phosphate salt of any of the preceding embodiments, wherein the phosphate salt is a 2:1 phosphate:Compound A salt.
GGG. The phosphate salt of any of the preceding embodiments, wherein the phosphate salt is a 1:2 phosphate:Compound A salt.
HHH. A besylate salt of Compound A:

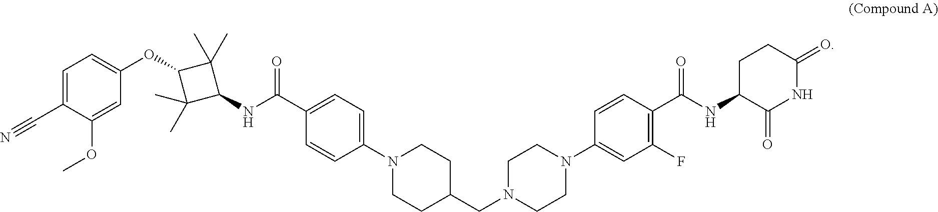 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

III. The besylate salt of embodiment 60, wherein the besylate salt is crystalline.
JJJ. The besylate salt of embodiment 60, wherein the besylate salt is amorphous.
KKK. The besylate salt of any of the preceding embodiments, wherein the besylate salt is a crystalline polymorphic form characterized by two or three XRPD signals selected from the group consisting of 18.5 °2θ, 18.3 °2θ, and 22.6 °2θ (±0.2 °2θ; ±0.1 °2θ; or ±0.0 °2θ; Cu Kα1 radiation).
LLL. The besylate salt of any of the preceding embodiments, wherein the besylate salt is a crystalline polymorphic form characterized by XRPD signals at 18.5 °2θ, 18.3 °2θ, and 22.6 °2θ (±0.2 °2θ; ±0.1 °2θ; or ±0.0 °2θ; Cu Kα1 radiation).
MMM. The besylate salt of any of the preceding embodiments, wherein the besylate salt is a crystalline polymorphic form characterized by an XRPD spectrum substantially similar to that shown in FIG. 97.
NNN. The besylate salt of any of the preceding embodiments, wherein the besylate salt is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty XRPD signals selected from those set forth in Table 12.
OOO. The besylate salt of any of the preceding embodiments, wherein the besylate salt is a 1:1 besylate:Compound A salt.
PPP. The besylate salt of any of the preceding embodiments, wherein the besylate salt is a 2:1 besylate:Compound A salt.
QQQ. The besylate salt of any of the preceding embodiments, wherein the besylate salt is a 1:2 besylate:Compound A salt.
RRR. A method of treating prostate cancer in a subject need thereof comprising administering to the subject a therapeutically effective amount of a solid form or salt of any one of the preceding embodiments.
SSS. The method of any one of the preceding embodiments, further comprising administering an effective amount of at least one additional anti-cancer agent to the subject.
TTT. The method of any one of the preceding embodiments, wherein the prostate cancer is metastatic prostate cancer.
UUU. The method of any one of the preceding embodiments, wherein the prostate cancer is castrate-resistant prostate cancer.
VVV. The method of any one of the preceding embodiments, wherein the prostate cancer is metastatic castrate-resistant prostate cancer.
WWW. The method of any one of the preceding embodiments, wherein the prostate cancer is castrate-sensitive prostate cancer.
XXX. The method of any one of the preceding embodiments, wherein the prostate cancer is metastatic castrate-sensitive prostate cancer.
YYY. The method of any one of the preceding embodiments, wherein the prostate cancer is prostate cancer naïve to novel hormonal agents (NHA).
ZZZ. The method of the previous claim, wherein the prostate cancer naïve to novel hormonal agents (NHA) has not been previously treated with a second generation antiandrogen.
AAAA. The method of any one of the preceding embodiments, wherein the metastatic prostate cancer is metastatic prostate cancer naïve to novel hormonal agents (NHA).
BBBB. The method of any one of the preceding embodiments, wherein the prostate cancer is castrate-resistant prostate cancer naïve to novel hormonal agents (NHA).
CCCC. The method of the previous claim, wherein the castrate-resistant prostate cancer naïve to novel hormonal agents (NHA) has not been previously treated with a second generation antiandrogen.
DDDD. The method of any one of the preceding embodiments, wherein the prostate cancer is castrate-sensitive prostate cancer naïve to novel hormonal agents (NHA).
EEEE. The method of the previous claim, wherein the castrate-sensitive prostate cancer naïve to novel hormonal agents (NHA) has not been previously treated with a second generation antiandrogen.
FFFF. The method of any one of the preceding embodiments, wherein the prostate cancer is metastatic castrate-resistant prostate cancer naïve to novel hormonal agents (NHA).
GGGG. The method of the previous claim, wherein the metastatic castrate-resistant prostate cancer naïve to novel hormonal agents (NHA) has not been previously treated with a second generation antiandrogen.
HHHH. The method of any one of the preceding embodiments, wherein the prostate cancer is metastatic castrate-sensitive prostate cancer naïve to novel hormonal agents (NHA).
IIII. The method of the previous claim, wherein the metastatic castrate-sensitive prostate cancer naïve to novel hormonal agents (NHA) has not been previously treated with a second generation antiandrogen.
JJJJ. The method of any one of the preceding embodiments, wherein the prostate cancer has not been previously treated with an androgen biosynthesis inhibitor or an androgen receptor blocker.
KKKK. The method of any one of the preceding embodiments, wherein prostate cancer has not been previously treated with abiraterone acetate.
LLLL. The method of any one of the preceding embodiments wherein prostate cancer has not been previously treated with an androgen receptor blocker selected from enzalutamide, darolutamide, and apalutamide.
MMMM. The method of any one of the preceding embodiments, wherein the subject has not been previously administered an androgen biosynthesis inhibitor or an androgen receptor blocker.
NNNN. The method of any one of the preceding embodiments, wherein subject has not been previously administered abiraterone acetate.
OOOO. The method of any one of the preceding embodiments wherein the subject has not been previously administered an androgen receptor blocker selected from enzalutamide, darolutamide, and apalutamide.

1. A solid form of Compound A: 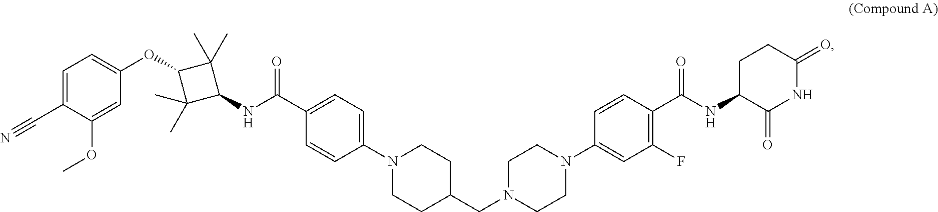 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
wherein the solid form is a crystalline polymorphic form characterized by two or three XRPD signals selected from the group consisting of 18.6 °2θ, 13.9 °2θ, and 15.3 °2θ (±0.2 °2θ Cu Kα1 radiation).

2. The solid form of claim 1, wherein the solid form is a crystalline polymorphic form characterized by XRPD signals at 18.6 °2θ, 13.9 °2θ, and 15.3 °2θ (±0.2 °2θ Cu Kα1 radiation).

3. A solid form of Compound A: 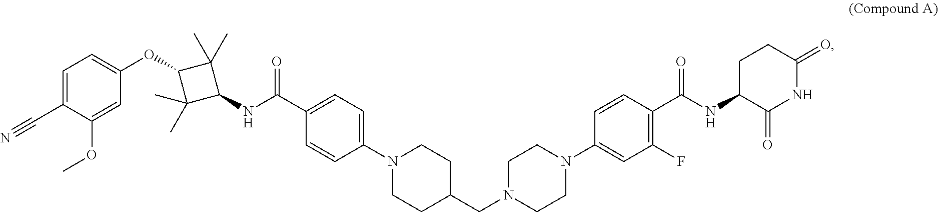 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
wherein the solid form is a crystalline polymorphic form characterized by an XRPD spectrum substantially similar to that shown in FIG. 80 or FIG. 86.

4. A solid form of Compound A: 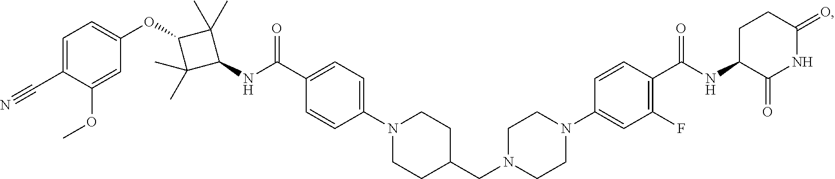 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, or forty-one XRPD signals selected from those set forth in Table 1.

5. A solid form of Compound A:

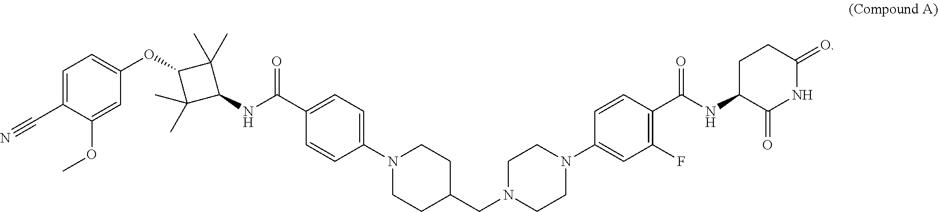 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

6. The solid form of claim 5, wherein the solid form is crystalline.

7. The solid form of claim 5, wherein the solid form is amorphous.

8. The solid form of claim 5, wherein the solid form is a crystalline polymorphic form characterized by two or three XRPD signals selected from the group consisting of 14.4 °2θ, 19.1 °2θ, and 15.8 °2θ (±0.2 °2θ Cu Kα1 radiation).

9. The solid form of claim 5, wherein the solid form is a crystalline polymorphic form characterized by an XRPD spectrum substantially similar to that shown in FIG. 87.

10. The solid form of claim 5, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, or forty XRPD signals selected from those set forth in Table 2.

11. The solid form of claim 5, wherein the solid form is a crystalline polymorphic form characterized by two or more, or three or more XRPD signals selected from the group consisting of 20.6 °2θ, 16.1 °2θ, 16.3 °2θ, 17.3 °2θ, and 16.8 °2θ (±0.2 °2θ Cu Kα1 radiation).

12. The solid form of claim 5, wherein the solid form is a crystalline polymorphic form characterized by an XRPD spectrum substantially similar to that shown in FIG. 88.

13. The solid form of claim 5, wherein the solid form is a crystalline polymorphic form characterized by one, two, three, four, five, six, seven, eight, nine, ten, eleven, twelve, thirteen, fourteen, fifteen, sixteen, seventeen, eighteen, or nineteen XRPD signals selected from those set forth in Table 3.

14. The solid form of claim 5, wherein the solid form is a crystalline polymorphic form characterized by two or three XRPD signals selected from the group consisting of 14.6 °2θ, 17.7 °2θ, and 16.7 °2θ (±0.2 °2θ Cu Kα1 radiation).

15. The solid form of claim 5, wherein the solid form is a crystalline polymorphic form characterized by an XRPD spectrum substantially similar to that shown in FIG. 89.

16. The solid form of claim 5, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four XRPD signals selected from those set forth in Table 4.

17. The solid form of claim 5, wherein the solid form is a crystalline polymorphic form characterized by two or three XRPD signals selected from the group consisting of 14.9 °2θ, 22.6 °2θ, and 7.1 °2θ (±0.2 °2θ Cu Kα1 radiation).

18. The solid form of claim 5, wherein the solid form is a crystalline polymorphic form characterized by an XRPD spectrum substantially similar to that shown in FIG. 90.

19. The solid form of claim 5, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, or thirty-five XRPD signals selected from those set forth in Table 5.

20. The solid form of claim 5, wherein the solid form is a crystalline polymorphic form characterized by two or three XRPD signals selected from the group consisting of 3.5 °2θ, 3.6 °2θ, and 15.7 °2θ (±0.2 °2θ Cu Kα1 radiation).

21. The solid form of claim 5, wherein the solid form is a crystalline polymorphic form characterized by an XRPD spectrum substantially similar to that shown in FIG. 91.

22. The solid form of claim 5, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, or thirty-six XRPD signals selected from those set forth in Table 6.

23. The solid form of claim 5, wherein the solid form is a crystalline polymorphic form characterized by two or three XRPD signals selected from the group consisting of 4.8 °2θ, 15.7 °2θ, and 17.9 °2θ (±0.2 °2θ Cu Kα1 radiation).

24. The solid form of claim 5, wherein the solid form is a crystalline polymorphic form characterized by an XRPD spectrum substantially similar to that shown in FIG. 92.

25. The solid form of claim 5, wherein the solid form is a crystalline polymorphic form characterized by one, two, three, four, five, six, seven, eight, nine, ten, eleven, twelve, thirteen, fourteen, fifteen, or sixteen XRPD signals selected from those set forth in Table 7.

26. The solid form of claim 5, wherein the solid form is a crystalline polymorphic form characterized by two or three XRPD signals selected from the group consisting of 4.9 °2θ, 15.9 °2θ, and 18.2 °2θ (±0.2 °2θ Cu Kα1 radiation).

27. The solid form of claim 5, wherein the solid form is a crystalline polymorphic form characterized by an XRPD spectrum substantially similar to that shown in FIG. 93.

28. The solid form of claim 5, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, or twenty-two XRPD signals selected from those set forth in Table 8.

29. The solid form of claim 5, wherein the solid form is a crystalline polymorphic form characterized by two or three XRPD signals selected from the group consisting of 17.2 °2θ, 21.0 °2θ, and 24.2 °2θ (±0.2 °2θ Cu Kα1 radiation).

30. The solid form of claim 5, wherein the solid form is a crystalline polymorphic form characterized by an XRPD spectrum substantially similar to that shown in FIG. 94.

31. The solid form of claim 5, wherein the solid form is a crystalline polymorphic form characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, or forty-two XRPD signals selected from those set forth in Table 9.

32. A tosylate salt of Compound A:

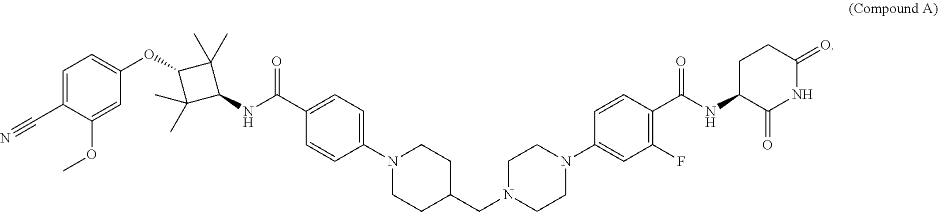 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

33. (canceled)

34. (canceled)

35. The tosylate salt of claim 32, wherein the tosylate salt is a crystalline polymorphic form characterized by two or three XRPD signals selected from the group consisting of 3.5 °2θ, 22.0 °2θ, and 23.0 °2θ (±0.2 °2θ Cu Kα1 radiation).

36. A phosphate salt of Compound A:

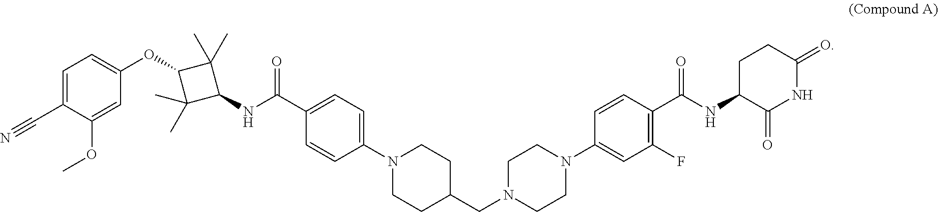 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

37. (canceled)

38. (canceled)

39. The phosphate salt of claim 36, wherein the phosphate salt is a crystalline polymorphic form characterized by two or three XRPD signals selected from the group consisting of 23.6 °2θ, 3.3 °2θ, and 19.9 °2θ (±0.2 °2θ Cu Kα1 radiation).

40. A besylate salt of Compound A:

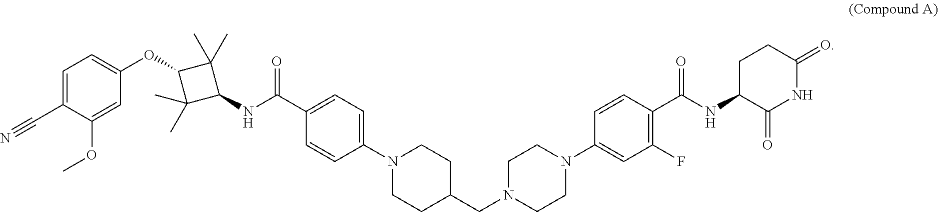 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

41. (canceled)

42. (canceled)

43. The besylate salt of claim 40, wherein the besylate salt is a crystalline polymorphic form characterized by two or three XRPD signals selected from the group consisting of 18.5 °2θ, 18.3 °2θ, and 22.6 °2θ (±0.2 °2θ Cu Kα1 radiation).

44. A method of treating prostate cancer in a subject need thereof comprising administering to the subject a therapeutically effective amount of a solid form or salt of claim 1.

45. The method of claim 44, wherein the prostate cancer is metastatic prostate cancer.

46. The method of claim 44, wherein the prostate cancer is castrate-resistant prostate cancer or metastatic castrate-resistant prostate cancer.

47. (canceled)

48. The method of claim 44, wherein the prostate cancer is castrate-sensitive prostate cancer or metastatic castrate-sensitive prostate cancer.

49. (canceled)

50. The method of claim 44, wherein the prostate cancer is prostate cancer naïve to novel hormonal agents (NHA).

51.-67. (canceled)
